(19) United States
(12) Patent Application Publication
(10) Pub. No.: US 2011/0287936 A1
(43) Pub. Date: Nov. 24, 2011

(54) GENE SWITCH COMPOSITIONS AND METHODS OF USE

(75) Inventors: Jin Fang, Redwood City, CA (US); William J. Gordon-Kamm, Urbandale, IA (US); Keith S. Lowe, Johnston, IA (US); Kevin E. McBride, Davis, CA (US); Brian McGonigle, Wilmington, DE (US); Carl R. Simmons, Des Moines, IA (US)

(73) Assignees: E.I. duPont de Nemours and Company, Wilmington, DE (US); Pioneer Hi-Bred International, Inc., Johnston, IA (US)

(21) Appl. No.: 13/086,765

(22) Filed: Apr. 14, 2011

Related U.S. Application Data
(60) Provisional application No. 61/327,172, filed on Apr. 23, 2010.

(51) Int. Cl. A01N 47/34; A01P 21/00; A01H 5/10; C12N 5/10; C12N 15/113; A01H 5/00

(52) U.S. Cl. 504/212; 504/329; 504/214; 504/211; 536/24.1; 800/298; 435/419; 800/320.1; 800/320.2; 800/320; 800/320.3; 800/312; 800/306; 800/314; 800/317.3; 800/322; 504/215

(57) ABSTRACT

Compositions and methods relating to the use of sulfonylurea-mediated control of gene expression are provided. Compositions include sulfonylurea responsive chemical switches wherein the gene expression is regulated by a sulfonylurea compound. Compositions also include polynucleotides encoding the polypeptides as well as constructs, vectors, prokaryotic and eukaryotic cells, and eukaryotic organisms including plants and seeds comprising the polynucleotide, and/or produced by the methods. Also provided are methods to regulate expression of a polynucleotide of interest in a cell or organism, and methods to modify a genome, including in a plant or plant cell.

### CROSS-REFERENCE TO RELATED APPLICATIONS

This application claims the benefit of U.S. Provisional Application No. 61/327,172, filed Apr. 23, 2010, which is incorporated herein in its entirety by reference.

### REFERENCE TO SEQUENCE LISTING SUBMITTED ELECTRONICALLY

The official copy of the sequence listing is submitted electronically via EFS-Web as an ASCII formatted sequence listing with a file named 403001seqlist.txt, created on Apr. 14, 2011, and having a size of 1.33 MB and is filed concurrently with the specification. The sequence listing contained in this ASCII formatted document is part of the specification and is herein incorporated by reference in its entirety.

### FIELD OF THE INVENTION

The invention relates to the field of molecular biology, more particularly to the regulation of gene expression.

### BACKGROUND

The tetracycline operon system, comprising repressor and operator elements, was originally isolated from bacteria. The operon system is tightly controlled by the presence of tetracycline, and self-regulates the level of expression of tetA and tetR genes. The product of tetA removes tetracycline from the cell. The product of tetR is the repressor protein that binds to the operator elements with a K_{d} of about 10 pM in the absence of tetracycline, thereby blocking expression or tetA and tetR.

This system has been modified to control expression of other polynucleotides of interest, and/or for use in other organisms, mainly for use in animal systems. Tet operon based systems have had limited use in plants, at least partially due to problems with the inducers which are typically antibiotic compounds, and sensitive to light.

There is a need to regulate expression of sequences of interest in organisms. Gene switch compositions and methods to regulate expression in response to compounds, such as sulfonylurea compounds, are provided.

### SUMMARY

Compositions and methods relating to the use of sulfonylurea-mediated control of gene expression are provided. Compositions include sulfonylurea responsive chemical switches wherein the gene expression is regulated by a sulfonylurea compound. Compositions also include polynucleotides encoding the polypeptides, repressible promoters, as well as constructs, vectors, prokaryotic and eukaryotic cells, and organisms including plants, plant cells, and seeds comprising any component, and/or produced by any method. Also provided are methods to regulate expression of a polynucleotide of interest in a cell or organism, and methods to modify a genome, including in a plant or plant cell.

The following embodiments are encompassed by the present invention.

1. A method to regulate expression in a plant cell contained in a plant or in a seed comprising:

(a) providing the plant cell comprising a sulfonylurea-regulated gene switch which controls expression of a polynucleotide of interest, wherein said plant cell is a sulfonylurea tolerant plant cell; and;

(b) providing a sulfonylurea compound which regulates the gene switch wherein the sulfonylurea compound is provided by foliar application, root drench application, pre-emergence application, post-emergence application, or seed treatment application.

2. The method of embodiment 1, wherein expression of the polynucleotide of interest alters the phenotype of the plant cell.

3. The method of embodiment 1 or 2, wherein expression of the polynucleotide of interest alters the genotype of the plant cell.

4. The method of any one of embodiments 1-3, wherein providing the sulfonylurea compound activates expression of the polynucleotide of interest.

5. The method of any one of embodiments 1-4, wherein the plant cell is from a monocot or a dicot.

6. The method of embodiment 5, wherein the plant cell is from maize, rice, sorghum, sugarcane, barley, oat, wheat, turfgrass, soybean, canola, cotton, tobacco, sunflower, safflower, or alfalfa.

7. The method of any one of embodiments 1-6, wherein the sulfonylurea-regulated gene switch comprises a repressible promoter operably linked to a polynucleotide of interest, wherein the repressible promoter comprises a tet operator.

8. The method of any one of embodiments 1-7, wherein the sulfonylurea-regulated gene switch comprises a repressible promoter selected from the group consisting of SEQ ID NO:855, 856, 857, 858, 859, and 860, or a repressible promoter having at least 95% sequence identity to SEQ ID NO:855, 856, 857, 858, 859, 860 or 862.

9. The method of any one of embodiments 1-8, wherein the sulfonylurea compound comprises a pyrimidinylsulfonylurea compound, a triazinylsulfonylurea compound, or a thiadazolylurea compound.

10. The method of embodiment 9, wherein the sulfonylurea compound comprises a chlorosulfuron, an ethametsulfuron, a thifensulfuron, a metsulfuron, a sulfometuron, a tribenuron, a chlorimuron, a nicosulfuron, or a rimsulfuron.

11. The method of any one of embodiments 1-10, wherein the polynucleotide of interest encodes a polypeptide that specifically binds to a target nucleic acid sequence.

12. The method of embodiment 11, wherein the polypeptide is a recombinase, an integrase, a nuclease, a homing endonuclease, or a zinc-finger nuclease.

13. The method of any one of embodiments 1-12, wherein the sulfonylurea-regulated gene switch comprises a sulfonylurea-responsive repressor comprising an amino acid sequence of any one of SEQ ID NOs:3-419, or an amino acid sequence having at least 85% sequence identity to any one of SEQ ID NOs:3-419.

14. A sulfonylurea-regulated gene switch which controls expression of a polynucleotide of interest, wherein the polynucleotide of interest encodes a polypeptide that specifically binds a DNA sequence or encodes a polypeptide that cuts a DNA sequence.

15. The sulfonylurea-regulated gene switch of embodiment 14, wherein the encoded polypeptide is a recombinase, an integrase, a nuclease, a homing endonuclease, or a zinc-finger nuclease.

16. The sulfonylurea-regulated gene switch of embodiment 14 or 15, wherein the sulfonylurea gene switch comprises a sulfonylurea responsive repressor, wherein the sulfonylurea responsive repressor is operably linked to a promoter active in the plant wherein the promoter is a constitutive promoter, a tissue-preferred promoter, a developmental stage-preferred promoter, an inducible promoter, or a repressible promoter.

17. The sulfonylurea-regulated gene switch of embodiment 16, wherein

(a) the constitutive promoter is a MTH promoter, an EF1a promoter, a PIP promoter, a ubiquitin promoter, an actin promoter, or a 35S CaMV promoter;

(b) the tissue-preferred promoter is a meristem-preferred promoter, an embryo-preferred promoter, a leaf-preferred promoter, a root-preferred promoter, an anther-preferred promoter, a pollen-preferred promoter, or a floral-preferred promoter;

(c) the developmental stage-preferred promoter is an early embryo promoter, a late embryo promoter, a germination-preferred promoter, or a senescence-preferred promoter;

(d) the inducible promoter is a chemical inducible promoter, a pathogen inducible promoter, a heat-stress promoter, a drought-stress promoter, a light inducible promoter, an osmoticum inducible promoter, or a metal inducible promoter; or,

(e) the repressible promoter is a tetracycline-repressible promoter, or a lactose-repressible promoter.

18. The sulfonylurea-regulated gene switch of embodiment 16 or 17, wherein the polynucleotide of interest or said sulfonylurea-responsive repressor is operably linked to a repressible promoter comprising at least one tetracycline operator sequence.

19. The sulfonylurea-regulated gene switch of embodiment 18, wherein the repressible promoter is a constitutive promoter, a tissue-preferred promoter, or a development stage-preferred promoter.

20. The sulfonylurea-regulated gene switch of embodiment 19, wherein the repressible promoter is a 35S CaMV promoter, an actin promoter, an EF1A promoter, an MMV promoter, a dMMV promoter, a MP1 promoter, or a BSV promoter.

21. The sulfonylurea regulated gene switch of embodiment 20, wherein the repressible promoter comprises a polynucleotide sequence as set forth in SEQ ID NO:855, 856, 857, 858, 859, 860 or 862, or a polynucleotide sequence having at least 95% sequence identity to SEQ ID NO:855, 856, 857, 858, 859, 860 or 862.

22. The sulfonylurea regulated gene switch of embodiment 16, wherein the sulfonylurea-responsive repressor comprises an amino acid sequence of any one of SEQ ID NOs:3-419, or an amino acid sequence having at least 85% sequence identity to any one of SEQ ID NOs:3-419.

23. A transgenic plant, a transgenic plant cell, or a transgenic seed comprising the sulfonylurea regulated gene switch of any one of embodiments 14-22.

24. The transgenic plant, the transgenic plant cell, or the transgenic seed of embodiment 23, wherein the transgenic plant, the transgenic plant cell, or the transgenic seed is from a monocot or a dicot.

25. The transgenic plant, the transgenic plant cell, or the transgenic seed of embodiment 24, wherein the monocot or dicot is from maize, rice, sorghum, sugarcane, barley, oat, wheat, turfgrass, soybean, canola, cotton, tobacco, sunflower, safflower, or alfalfa.

26. A recombinant polynucleotide comprising a repressible promoter, wherein the repressible promoter is active in a plant cell, and the repressible promoter comprises an actin promoter, an EF1A promoter, an MMV promoter, a dMMV promoter, an MP1 promoter, or a BSV promoter operably linked to at least one operator sequence.

27. The recombinant polynucleotide of embodiment 26, wherein the repressible promoter comprises a polynucleotide sequence as set forth in SEQ ID NO:855, 856, 857, 858, 859, 860 or 862 or a polynucleotide sequence having at least 95% sequence identity to SEQ ID NO:855, 856, 857, 858, 859, 860 or 862.

28. A method to regulate expression in a cell comprising:

(a) providing the cell comprising a regulated gene switch which controls expression of a polynucleotide of interest, wherein the gene switch comprises a repressible promoter of embodiment 26 or 27; and;

(b) providing a ligand compound which regulates the gene switch, wherein ligand compound comprises a tetracycline, a sulfonylurea, or any analogs thereof.

29. A recombinant polynucleotide comprising a repressible promoter operably linked to a polynucleotide encoding a sulfonylurea-responsive repressor.

30. The recombinant polynucleotide of embodiment 29, wherein the encoded sulfonylurea-responsive repressor comprises an amino acid sequence of any one of SEQ ID NOs:3-419 or an amino acid sequence having at least 85% sequence identity to any one of SEQ ID NOs:3-419.

31. The recombinant polynucleotide of embodiment 29, wherein the repressible promoter is an actin promoter, an MMV promoter, a dMMV promoter, an MP1 promoter, or a BSV promoter operably linked to at least one operator sequence.

32. The recombinant polynucleotide of any one of embodiments 29-31, wherein the repressible promoter comprises a polynucleotide sequence as set forth in SEQ ID NO:855, 856, 857, 858, 859, 860 or 862, or a polynucleotide sequence having at least 95% sequence identity to SEQ ID NO:855, 856, 857, 858, 859, 860 or 862.

### BRIEF DESCRIPTION OF DRAWINGS

FIG. 1. Summary of source diversity, library design, hit diversity, and population bias for several generations of sulfonylurea repressor shuffling libraries. A dash (“-”) indicates no amino acid diversity introduced at that position in that library. An X indicates that the library oligonucleotides were designed to introduce complete amino acid diversity (any of 20 amino acids) at that position in that library. Residues in bold indicate bias during selection with larger font size indicating a greater degree of bias in the selected population. Residues in parentheses indicate selected mutations. The phylogenetic diversity pool was derived from a broad family of 34 tetracycline repressor sequences.

FIG. 2. Exemplifies promoter:operator designs. Panel A shows a dMMV promoter before and after placement of tet operators into the sequence. Panel B shows EF1A2 promoter before and after placement of tet operators into the sequence.

FIG. 3. Exemplifies promoter activity before and after addition of tet operators. The y-axis of each graph is relative light units measured by the luciferase assay. Panel A compares 35SCaMV promoter +/−tet operators. Panel B compares MMV promoter +/−tet operators. Panel C compares EF1A2 promoter +/−tet operators. Panel D compares dMMV promoter +/−tet operators.

FIG. 4. Exemplifies repressor and/or ligand regulation of tet operator-containing promoters. The y-axis of each graph is relative light units measured by the luciferase assay.

FIG. 5. Examples of gene switch elements, compositions, and combinations are provided. Pro indicates any promoter, Pro/Op indicates any repressible promoter, POI is a polynucleotide of interest, and RS indicates a recombination site.

FIG. 6. Exemplary autoregulatory constructs.

FIG. 7. Transient expression of control and autoregulatory constructs.

FIG. 8. Transient expression of control and autoregulatory constructs.

FIG. 9. Dose response of dsRED activation to ethametsulfuron in transgenic auto-regulated lines of tobacco.

### DETAILED DESCRIPTION

Chemically regulated expression tools have proven valuable for studying gene function and regulation in many biological systems. These systems allow testing for the effect of expression of any gene of interest in a culture system or whole organism when the transgene cannot be specifically regulated, or continuously expressed due to negative consequences. These systems essentially provide the opportunity to do “pulse” or “pulse-chase” gene expression testing. A chemical switch-mediated expression system allows testing of genomic, proteomic, and/or metabolomic responses immediately following activation or inactivation of a target sequence. These types of tests cannot be easily done with constitutive, developmental, or tissue-specific expression systems. Chemical switch technologies may also provide a means for gene therapy.

Chemical switch systems can be commercially applied, such as in agricultural biotechnology. For agricultural purposes it is desired to be able to control the expression and/or genetic flow of transgenes in the environment, such as herbicide resistance genes, especially in cases where weedy relatives of the target crop exist. In addition, having a family of viable chemical switch mechanisms would enable trait inventory management from a single transgenic crop, for example, one production line could be used to deliver selected traits on customer demand via specific chemical activation. Additionally, hybrid seed production could be streamlined by using chemical control of hybrid maintenance.

The Tet repressor (TetR) based genetic switch system widely used in animal systems has had limited use in plant genetic systems, due in part to problems with the activator ligands. A tetracycline repressor has been redesigned to specifically recognize sulfonylurea compounds instead of tetracycline compounds, while retaining the ability to specifically bind tetracycline operator sequences. Through several rounds of library design and shuffling based on rational modeling, sulfonylurea-responsive repressors (SuRs) have been developed. Compositions and methods relating to the use of sulfonylurea-responsive repressors are provided.

A chemical switch, or gene switch, comprises two components. One component comprises a polynucleotide encoding a repressor, the second component comprises a repressible/inducible promoter operably linked to a polynucleotide of interest. Expression of the polynucleotide of interest is optionally controlled by providing the appropriate chemical ligand. The repressible/ inducible promoter (hereafter referred to as a repressible promoter) comprises at least one operator sequence to which the repressor polypeptide specifically binds, which controls the transcriptional activity of the promoter. Useful repressors include those that specifically bind to an operator in the absence of the chemical ligand, those with a reverse phenotype that specifically bind to an operator in the presence of the chemical ligand, and those fused to an activator or domain to control activity.

The activity of the gene switch can be controlled by selecting the combination of elements used in the switch. These include, but are not limited to the promoter operably linked to the repressor, the repressor, the repressible promoter operably linked to the polynucleotide of interest, and optionally the polynucleotide of interest. Further control is provided by selection, dosage, conditions, and/or timing of the application of the chemical ligand. In some examples the expression of the polynucleotide of interest can be controlled more stringently, controlled in various tissues or cells, restricted to selected tissue or cell type, restricted to specific developmental stage(s), restricted to specific environmental conditions, and/or restricted to specific generation of a plant or progeny thereof. In some examples the repressor is operably linked to a constitutive promoter. In some examples the repressor is operably linked to a non-constitutive promoter, including but not limited to a tissue preferred promoter, an inducible promoter, a repressible promoter, a developmental stage preferred promoter, or a promoter having more than one of these properties. In some examples the promoter is primarily expressed in roots, leaves, stems, flowers, silks, anthers, pollen, meristem, germline, seed, endosperm, embryos, or progeny.

In some examples the gene switch may further comprise additional elements. In some examples, one or more additional elements may provide means by which expression of the polynucleotide of interest can be controlled more stringently, controlled in various tissues or cells, restricted to selected tissue or cell type, restricted to specific developmental stage(s), restricted to specific environmental conditions, and/or restricted to specific generation of a plant or progeny thereof. In some examples those elements include site-specific recombination sites, site-specific recombinases, or combinations thereof.

In some examples, the gene switch may comprise a polynucleotide encoding a repressor, a promoter linked to a polynucleotide of interest, a sequence flanked by site-specific recombination sites, and a repressible promoter operably linked to a site-specific recombinase that specifically recognizes the site-specific recombination sites and implements a recombination event. In some examples, the recombination event is excision of the sequence flanked by the recombination sites. In some instances, the excision creates an operable linkage between the promoter and the polynucleotide of interest. In some examples, the promoter operably linked to the polynucleotide of interest is a non-constitutive promoter, including but not limited to a tissue preferred promoter, an inducible promoter, a repressible promoter, a developmental stage preferred promoter, or a promoter having more than one of these properties. In some examples the promoter is primarily expressed in roots, leaves, stems, flowers, silks, anthers, pollen, meristem, germline, seed, endosperm, embryos, or progeny.

For example, the gene switch may comprise a polynucleotide encoding a repressor, a repressible promoter linked to a polynucleotide of interest, a sequence flanked by site-specific recombination sites, and a site-specific recombinase that specifically recognizes the site-specific recombination sites and implements a recombination event. In some examples, the recombination event is excision of the sequence flanked by the recombination sites. In some instances, the excision creates an operable linkage between the repressible promoter and the polynucleotide of interest. In some examples, the sequence flanked by recombination sites comprises a recombinase expression cassette. In some examples the promoter is primarily expressed in roots, leaves, stems, flowers, silks, anthers, pollen, meristem, germline, seed, endosperm, or embryos. In some examples the excision occurs in a parent, a cell, a tissue, or a tissue culture such that the progeny inherit the post-excision product.

In some examples, the gene switch may comprise a polynucleotide encoding a repressor, a promoter operably linked to a polynucleotide of interest flanked by site-specific recombination sites, and a repressible promoter operably linked to a site-specific recombinase that specifically recognizes the site-specific recombination sites and implements a recombination event. In some examples, the recombination event is excision of the sequence flanked by the recombination sites. In some examples, the promoter operably linked to the polynucleotide of interest is a non-constitutive promoter, including but not limited to a tissue preferred promoter, an inducible promoter, a repressible promoter, a developmental stage preferred promoter, or a promoter having more than one of these properties. In some examples the promoter is primarily expressed in roots, leaves, stems, flowers, silks, anthers, pollen, meristem, germline, seed, endosperm, embryos, or progeny.

In another example, the gene switch may comprise a polynucleotide encoding a repressor, a promoter linked to a polynucleotide of interest, a sequence flanked by site-specific recombination sites, and a repressible promoter operably linked to a site-specific recombinase that specifically recognizes the site-specific recombination sites and implements a recombination event. In some examples, the recombination event is inversion of the sequence flanked by the recombination sites. In some instances, the inversion creates an operable linkage between the promoter and the polynucleotide of interest. In some examples, the promoter operably linked to the polynucleotide of interest is a non-constitutive promoter, including but not limited to a tissue preferred promoter, an inducible promoter, a repressible promoter, a developmental stage preferred promoter, or a promoter having more than one of these properties. In some examples the promoter is primarily expressed in roots, leaves, stems, flowers, silks, anthers, pollen, meristem, germline, seed, endosperm, embryos, or progeny. In some examples the inversion occurs in a parent, a cell, a tissue, or a tissue culture such that the progeny inherit the post-inversion product.

For example, the gene switch may comprise a polynucleotide encoding a repressor, a repressible promoter linked to a polynucleotide of interest, a sequence flanked by site-specific recombination sites, and a site-specific recombinase that specifically recognizes the site-specific recombination sites and implements a recombination event. In some examples, the recombination event is inversion of the sequence flanked by recombination sites. In some instances, the inversion creates an operable linkage between the repressible promoter and the polynucleotide of interest. In some cases, the sequence flanked by site-specific recombination sites is the polynucleotide of interest. In some cases, the sequence flanked by site-specific recombination sites is the repressible promoter. In some examples, a recombinase expression cassette is provided, wherein the recombinase is operably linked to a non-constitutive promoter, including but not limited to a tissue preferred promoter, an inducible promoter, a repressible promoter, a developmental stage preferred promoter, or a promoter having more than one of these properties. In some examples the promoter is primarily expressed in roots, leaves, stems, flowers, silks, anthers, pollen, meristem, seed, endosperm, embryos, or progeny. In some examples the inversion occurs in a parent, a cell, a tissue, or a tissue culture such that the progeny inherit the post-inversion product.

In some examples, the gene switch may comprise a polynucleotide encoding a repressor, a promoter operably linked to a polynucleotide of interest flanked by site-specific recombination sites, and a repressible promoter operably linked to a site-specific recombinase that specifically recognizes the site-specific recombination sites and implements a recombination event. In some examples, the recombination event is inversion of the sequence flanked by the recombination sites. In some examples, the promoter operably linked to the polynucleotide of interest is a non-constitutive promoter, including but not limited to a tissue preferred promoter, an inducible promoter, a repressible promoter, a developmental stage preferred promoter, or a promoter having more than one of these properties. In some examples the promoter is primarily expressed in roots, leaves, stems, flowers, silks, anthers, pollen, meristem, germline, seed, endosperm, embryos, or progeny.

Sulfonylurea-responsive repressors (SuRs) include any repressor polypeptide whose binding to an operator sequence is controlled by a ligand comprising a sulfonylurea compound. In some examples, the repressor binds specifically to the operator in the absence of a sulfonylurea ligand. In some examples, the repressor binds specifically to the operator in the presence of a sulfonylurea ligand. Repressors that bind to an operator in the presence of the ligand are sometimes called a reverse repressor. In some examples compositions include SuR polypeptides that specifically bind to a tetracycline operator, wherein the specific binding is regulated by a sulfonylurea compound. In some examples compositions include an isolated sulfonylurea repressor (SuR) polypeptide comprising at least one amino acid substitution to a wild type tetracycline repressor protein ligand binding domain wherein the SuR polypeptide, or a multimer thereof, specifically binds to a polynucleotide comprising an operator sequence, wherein repressor-operator binding is regulated by the absence or presence of a sulfonylurea compound. In some examples compositions included isolated sulfonylurea repressors comprising a ligand binding domain comprising at least one amino acid substitution to a wild type tetracycline repressor protein ligand binding domain fused to a heterologous operator DNA binding domain which specifically binds to a polynucleotide comprising the operator sequence or derivative thereof, wherein repressor-operator binding is regulated by the absence or presence of a sulfonylurea compound. Any operator DNA binding domain can be used, including but not limited to an operator DNA binding domain from repressors included tet, lac, trp, phd, arg, LexA, phiCh1 repressor, lambda C1 and Cro repressors, phage X repressor, MetJ, phir1t rro, phi434 C1 and Cro repressors, RafR, gal, ebg, uxuR, exuR, ROS, SinR, PurR, FruR, P22 C2, TetC, AcrR, Betl, Bm3R1, EnvR, QacR, MtrR, TcmR, Ttk, YbiH, YhgD, and mu Ner, or DNA binding domains in Interpro families including but not limited to IPR001647, IPR010982, and IPR011991.

In some examples compositions include an isolated sulfonylurea repressor (SuR) polypeptides comprising at least one amino acid substitution to a wild type tetracycline repressor protein wherein the SuR polypeptide, or a multimer thereof, specifically binds to a polynucleotide comprising a tetracycline operator sequence, wherein repressor-operator binding is regulated by the absence or presence of a sulfonylurea compound.

Wild type repressors include tetracycline class A, B, C, D, E, G, H, J and Z repressors. An example of the TetR(A) class is found on the Tn1721 transposon and deposited under GenBank accession X61307, crossreferenced under gi48198, with encoded protein accession CAA43639, crossreferenced under gi48195 and UniProt accession Q56321. An example of the TetR(B) class is found on the Tn10 transposon and deposited under GenBank accession X00694, crossreferenced under gi43052, with encoded protein accession CAA25291, crossreferenced under gi43052 and UniProt accession PO4483. An example of the TetR(C) class is found on the pSC101 plasmid and deposited under GenBank Accession M36272, crossreferenced under gi150945, with encoded protein accession AAA25677, crossreferenced under gi150946. An example of the TetR(D) class is found in Salmonella ordonez and deposited under GenBank Accession X65876, crossreferenced under gi49073, with encoded protein accession CAA46707, crossreferenced under gi49075 and UniProt accessions POACT5 and P09164. An example of the TetR(E) class was isolated from E. coli transposon Tn10 and deposited under GenBank Accession M34933, crossreferenced under gi155019, with encoded protein accession AAA98409, crossreferenced under gi155020. An example of the TetR(G) class was isolated from Vibrio anguillarium and deposited under GenBank Accession S52438, crossreferenced under gi262928, with encoded protein accession AAB24797, crossreferenced under gi262929. An example of the TetR(H) class is found on plasmid pMV111 isolated from Pasteurella multocida and deposited under GenBank Accession 000792, crossreferenced under gi392871, with encoded protein accession AAC43249, crossreferenced under gi392872. An example of the TetR(J) class was isolated from Proteus mirabilis and deposited under GenBank Accession AF038993, crossreferenced under gi4104704, with encoded protein accession AAD12754, crossreferenced under gi4104706. An example of the TetR(Z) class was found on plasmid pAGI isolated from Corynebacterium glutamicum and deposited under GenBank Accession AF121000, crossreferenced under gi4583389, with encoded protein accession AAD25064, crossreferenced under gi4583390. In some examples the wild type tetracycline repressor is a class B tetracycline repressor protein. In some examples the wild type tetracycline repressor is a class D tetracycline repressor protein.

In some examples the sulfonylurea repressor (SuR) polypeptides comprise an amino acid substitution in the ligand binding domain of a wild type tetracycline repressor protein. In class B and D wild type TetR proteins, amino acid residues 6-52 represent the DNA binding domain. The remainder of the protein is involved in ligand binding and subsequent allosteric modification. For class B TetR residues 53-207 represent the ligand binding domain, while residues 53-218 comprise the ligand binding domain for the class D TetR. In some examples the SuR polypeptides comprise an amino acid substitution in the ligand binding domain of a wild type TetR(B) protein. In some examples the SuR polypeptides comprise an amino acid substitution in the ligand binding domain of a wild type TetR(B) protein of SEQ ID NO:1.

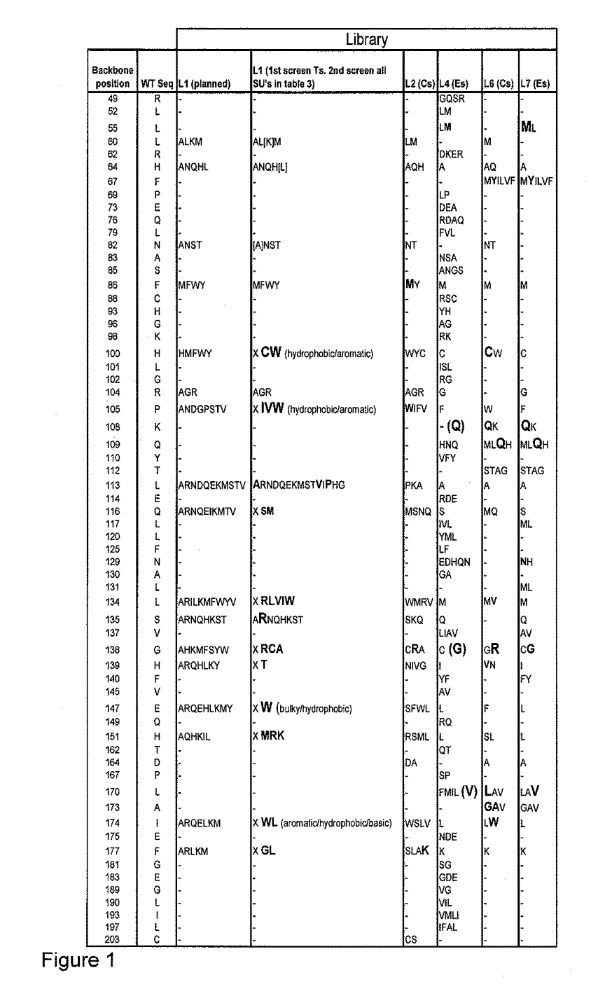

In some examples the isolated SuR polypeptides comprise an amino acid, or any combination of amino acids, corresponding to equivalent amino acid positions selected from the amino acid diversity shown in FIG. 1, wherein the amino acid residue position shown in FIG. 1 corresponds to the amino acid numbering of a wild type TetR(B). In some examples the isolated SuR polypeptides comprise a ligand binding domain comprising at least 10%, 20%, 30%, 40%, 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the amino acid residues shown in FIG. 1, wherein the amino acid residue position corresponds to the equivalent position using the amino acid numbering of a wild type TetR(B). In some examples the isolated SuR polypeptides comprise at least 10%, 20%, 30%, 40%, 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the amino acid residues shown in FIG. 1, wherein the amino acid residue position corresponds to the equivalent position using the amino acid numbering of a wild type TetR(B). In some examples the wild type TetR(B) is SEQ ID NO:1.

In some examples the isolated SuR polypeptide comprises a ligand binding domain comprising an amino acid substitution at a residue position selected from the group consisting of position 55, 60, 64, 67, 82, 86, 100, 104, 105, 108, 113, 116, 134, 135, 138, 139, 147, 151, 170, 173, 174, 177 and any combination thereof, wherein the amino acid residue position and substitution corresponds to the equivalent position using the amino acid numbering of a wild type TetR(B). In some examples the isolated SuR polypeptide further comprises an amino acid substitution at a residue position selected from the group consisting of 109, 112, 117, 131, 137, 140, 164 and any combination thereof. In some examples the wild type TetR(B) is SEQ ID NO:1.

In some examples the isolated SuR polypeptide has at least about 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the ligand binding domain of a wild type TetR(B) exemplified by amino acid residues 53-207 of SEQ ID NO:1, wherein the sequence identity is determined over the full length of the ligand binding domain using a global alignment method. In some examples the global alignment method uses the GAP algorithm with default parameters for an amino acid sequence % identity and % similarity using GAP Weight of 8 and Length Weight of 2, and the BLOSUM62 scoring matrix.

In some examples the isolated SuR polypeptide has at least about 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to a wild type TetR(B) exemplified by SEQ ID NO:1, wherein the sequence identity is determined over the full length of the polypeptide using a global alignment method. In some examples the global alignment method uses the GAP algorithm with default parameters for an amino acid sequence % identity and % similarity using GAP Weight of 8 and Length Weight of 2, and the BLOSUM62 scoring matrix.

Compositions include isolated SuR polypeptides having at least about 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the ligand binding domain of a SuR polypeptide selected from the group consisting of SEQ ID NO:3-419, wherein the sequence identity is determined over the full length of the ligand binding domain using a global alignment method. In some examples the global alignment method uses the GAP algorithm with default parameters for an amino acid sequence % identity and % similarity using GAP Weight of 8 and Length Weight of 2, and the BLOSUM62 scoring matrix.

In some examples the isolated SuR polypeptide have at least about 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to a SuR polypeptide selected from the group consisting of SEQ ID NO:3-419, wherein the sequence identity is determined over the full length of the polypeptide using a global alignment method. In some examples the global alignment method uses the GAP algorithm with default parameters for an amino acid sequence % identity and % similarity using GAP Weight of 8 and Length Weight of 2, and the BLOSUM62 scoring matrix.

In some examples the SuR polypeptides comprise an amino acid sequence that can be optimally aligned with a polypeptide sequence of L7-1A04 (SEQ ID NO:220), L1-22 (SEQ ID NO:7), L1-29 (SEQ ID NO:10), L1-02 (SEQ ID NO:3), L1-07 (SEQ ID NO:4), L1-20 (SEQ ID NO:6), L1-44 (SEQ ID NO:13), L6-3A09 (SEQ ID NO:402), L6-3H02 (SEQ ID NO:94), L7-4E03 (SEQ ID NO:403), L10-84(B12) (SEQ ID NO:404), or L13-46 (SEQ ID NO:405) to generate a BLAST bit score of at least 200, 250, 275, 300, 325, 350, 375, 400, 425, 450, 475, 500, 525, 550, 575, 600, 625, 650, 675, 700, or 750, wherein the BLAST alignment used the BLOSUM62 matrix, a gap existence penalty of 11, and a gap extension penalty of 1. In some examples the SuR polypeptides comprise an amino acid sequence that can be optimally aligned with a polypeptide sequence of L7-1A04 (SEQ ID NO:220) to generate a BLAST bit score of at least 374, optimally aligned with a polypeptide sequence of L1-22 (SEQ ID NO:7) to generate a BLAST bit score of at least 387, optimally aligned with a polypeptide sequence of L1-29 (SEQ ID NO:10) to generate a BLAST bit score of at least 393, optimally aligned with a polypeptide sequence of L1-07 (SEQ ID NO:4) to generate a BLAST bit score of at least 388, optimally aligned with a polypeptide sequence of L6-3A09 (SEQ ID NO:402) to generate a BLAST bit score of at least 381, optimally aligned with a polypeptide sequence of L7-4E03 (SEQ ID NO:403) to generate a BLAST bit score of at least 368, or optimally aligned with a polypeptide sequence of L13-46 (SEQ ID NO:405) to generate a BLAST bit score of at least 320, wherein the BLAST alignment used the BLOSUM62 matrix, a gap existence penalty of 11, and a gap extension penalty of 1. In some examples the SuR polypeptides comprise an amino acid sequence that can be optimally aligned with a polypeptide sequence of L7-1A04 (SEQ ID NO:220), L1-22 (SEQ ID NO:7), L1-29 (SEQ ID NO:10), L1-02 (SEQ ID NO:3), L1-07 (SEQ ID NO:4), L1-20 (SEQ ID NO:6), L1-44 (SEQ ID NO:13), L6-3A09 (SEQ ID NO:402), L6-3H02 (SEQ ID NO:94), L7-4E03 (SEQ ID NO:403), L10-84(B12) (SEQ ID NO:404), or L13-46 (SEQ ID NO:405) to generate a percent sequence identity of at least 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity, wherein the sequence identity is determined by BLAST alignment using the BLOSUM62 matrix, a gap existence penalty of 11, and a gap extension penalty of 1. In some examples the SuR polypeptides comprise an amino acid sequence that can be optimally aligned with a polypeptide sequence of L7-1A04 (SEQ ID NO:220) to generate a percent sequence identity of at least 88% sequence identity, optimally aligned with a polypeptide sequence of L1-22 (SEQ ID NO:7) to generate a percent sequence identity of at least 92% sequence identity, optimally aligned with a polypeptide sequence of L1-07 (SEQ ID NO:4) to generate a percent sequence identity of at least 93% sequence identity, optimally aligned with a polypeptide sequence of L1-20 (SEQ ID NO:6) to generate a percent sequence identity of at least 93% sequence identity, optimally aligned with a polypeptide sequence of L1-44 (SEQ ID NO:13) to generate a percent sequence identity of at least 93% sequence identity, optimally aligned with a polypeptide sequence of L6-3H02 (SEQ ID NO:94) to generate a percent sequence identity of at least 90% sequence identity, optimally aligned with a polypeptide sequence of L10-84(B12) (SEQ ID NO:404) to generate a percent sequence identity of at least 86% sequence identity, or optimally aligned with a polypeptide sequence of L13-46 (SEQ ID NO:405) to generate a percent sequence identity of at least 86% sequence identity, wherein the sequence identity is determined by BLAST alignment using the BLOSUM62 matrix, a gap existence penalty of 11, and a gap extension penalty of 1. In some examples the percent identity is determined using a global alignment method using the GAP algorithm with default parameters for an amino acid sequence % identity and % similarity using GAP Weight of 8 and Length Weight of 2, and the BLOSUM62 scoring matrix. In some examples the SuR polypeptides comprise an amino acid sequence that can be optimally aligned with a polypeptide sequence of L7-1A04 (SEQ ID NO:220), L1-22 (SEQ ID NO:7), L1-29 (SEQ ID NO:10), L1-02 (SEQ ID NO:3), L1-07 (SEQ ID NO:4), L1-20 (SEQ ID NO:6), L1-44 (SEQ ID NO:13), L6-3A09 (SEQ ID NO:402), L6-3H02 (SEQ ID NO:94), L7-4E03 (SEQ ID NO:403), L10-84(B12) (SEQ ID NO:404), or L13-46 (SEQ ID NO:405) to generate a BLAST similarity score of at least 400, 425, 450, 475, 500, 525, 550, 575, 600, 625, 650, 675, 700, 750, 800, 850, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, or 1200 wherein the BLAST alignment used the BLOSUM62 matrix, a gap existence penalty of 11, and a gap extension penalty of 1. In some examples the SuR polypeptides comprise an amino acid sequence that can be optimally aligned with a polypeptide sequence of L1-29 (SEQ ID NO:10) to generate a BLAST similarity score of at least 1006, optimally aligned with a polypeptide sequence of L1-07 (SEQ ID NO:4) to generate a BLAST similarity score of at least 996, optimally aligned with a polypeptide sequence of L6-3A09 (SEQ ID NO:402) to generate a BLAST similarity score of at least 978, optimally aligned with a polypeptide sequence of L7-4E03 (SEQ ID NO:403) to generate a BLAST similarity score of at least 945, or optimally aligned with a polypeptide sequence of L13-46 (SEQ ID NO:405) to generate a BLAST similarity score of at least 819, wherein the BLAST alignment used the BLOSUM62 matrix, a gap existence penalty of 11, and a gap extension penalty of 1. In some examples the SuR polypeptides comprise an amino acid sequence that can be optimally aligned with a polypeptide sequence of L7-1A04 (SEQ ID NO:220), L1-22 (SEQ ID NO:7), L1-29 (SEQ ID NO:10), L1-02 (SEQ ID NO:3), L1-07 (SEQ ID NO:4), L1-20 (SEQ ID NO:6), L1-44 (SEQ ID NO:13), L6-3A09 (SEQ ID NO:402), L6-3H02 (SEQ ID NO:94), L7-4E03 (SEQ ID NO:403), L10-84(B12) (SEQ ID NO:404), or L13-46 (SEQ ID NO:405) to generate a BLAST e-value score of at least e-60, e-70, e-75, e-80, e-85, e-90, e-95, e-100, e-105, e-106, e-107, e-108, e-109, e-110, e-111, e-112, e-113, e-114, e-115, e-116, e-117, e-118, e-119, e-120, or e-125, wherein the BLAST alignment used the BLOSUM62 matrix, a gap existence penalty of 11, and a gap extension penalty of 1. In some examples the SuR polypeptides comprise an amino acid sequence that can be optimally aligned with a polypeptide sequence of L1-02 (SEQ ID NO:3) to generate a BLAST e-value score of at least e-112, optimally aligned with a polypeptide sequence of L1-07 (SEQ ID NO:4) to generate a BLAST e-value score of at least e-111 , optimally aligned with a polypeptide sequence of L1-20 (SEQ ID NO:6) to generate a BLAST e-value score of at least e-111 , optimally aligned with a polypeptide sequence of L6-3A09 (SEQ ID NO:402) to generate a BLAST e-value score of at least e-108, optimally aligned with a polypeptide sequence of L7-4E03 (SEQ ID NO:403) to generate a BLAST e-value score of at least e-105, or optimally aligned with a polypeptide sequence of L13-46 (SEQ ID NO:405) to generate a BLAST e-value score of at least e-90 ,wherein the BLAST alignment used the BLOSUM62 matrix, a gap existence penalty of 11, and a gap extension penalty of 1. In some examples the polypeptide is selected from the group consisting of SEQ ID NO:3-419.

In some examples the isolated SuR polypeptides comprise a ligand binding domain from a polypeptide selected from the group consisting of SEQ ID NO:3-419. In some examples the isolated SuR polypeptides comprise an amino acid sequence selected from the group consisting of SEQ ID NO:3-419. In some examples the isolated SuR polypeptide is selected from the group consisting of SEQ ID NO:3-419, and the sulfonylurea compound is selected from the group consisting of a chlorsulfuron, an ethametsulfuron, a metsulfuron, a sulfometuron, a tribenuron, a chlorimuron, a nicosulfuron, a rimsulfuron and a thifensulfuron.

In some examples the isolated SuR polypeptides have an equilibrium binding constant for a sulfonylurea compound greater than 0.1 nM and less than 10 μM. In some examples the isolated SuR polypeptide has an equilibrium binding constant for a sulfonylurea compound of at least 0.1 nM, 0.5 nM, 1 nM, 10 nM, 50 nM, 100 nM, 250 nM, 500 nM, 750 nM, 1 μM, 5 μM, 7 μM but less than 10 μM. In some examples the isolated SuR polypeptide has an equilibrium binding constant for a sulfonylurea compound of at least 0.1 nM, 0.5 nM, 1 nM, 10 nM, 50 nM, 100 nM, 250 nM, 500 nM, 750 nM but less than 1 μM. In some examples the isolated SuR polypeptide has an equilibrium binding constant for a sulfonylurea compound greater than 0 nM, but less than 0.1 nM, 0.5 nM, 1 nM, 10 nM, 50 nM, 100 nM, 250 nM, 500 nM, 750 nM, 1 μM, 5 μM, 7 μM or 10 μM. In some examples the sulfonylurea compound is a chlorsulfuron, an ethametsulfuron, a metsulfuron, a sulfometuron, a tribenuron, a chlorimuron, a nicosulfuron, a rimsulfuron and/or a thifensulfuron.

In some examples the isolated SuR polypeptides have an equilibrium binding constant for an operator sequence greater than 0.1 nM and less than 10 μM. In some examples the isolated SuR polypeptide has an equilibrium binding constant for an operator sequence of at least 0.1 nM, 0.5 nM, 1 nM, 10 nM, 50 nM, 100 nM, 250 nM, 500 nM, 750 nM, 1 μM, 5 μM, 7 μM but less than 10 μM. In some examples the isolated SuR polypeptide has an equilibrium binding constant for an operator sequence of at least 0.1 nM, 0.5 nM, 1 nM, 10 nM, 50 nM, 100 nM, 250 nM, 500 nM, 750 nM but less than 1 μM. In some examples the isolated SuR polypeptide has an equilibrium binding constant for an operator sequence greater than 0 nM, but less than 0.1 nM, 0.5 nM, 1 nM, 10 nM, 50 nM, 100 nM, 250 nM, 500 nM, 750 nM, 1 μM, 5 μM, 7 μM or 10 μM. In some examples the operator sequence is a Tet operator sequence. In some examples the Tet operator sequence is a TetR(A) operator sequence, a TetR(B) operator sequence, a TetR(D) operator sequence, TetR(E) operator sequence, a TetR(H) operator sequence, or a functional derivative thereof.

The isolated SuR polypeptides specifically bind to a sulfonylurea compound. Sulfonylurea molecules comprise a sulfonylurea moiety (—S(O)2NHC(O)NH(R)—). In sulfonylurea herbicides the sulfonyl end of the sulfonylurea moiety is connected either directly or by way of an oxygen atom or an optionally substituted amino or methylene group to a typically substituted cyclic or acyclic group. At the opposite end of the sulfonylurea bridge, the amino group, which may have a substituent such as methyl (R being CH3) instead of hydrogen, is connected to a heterocyclic group, typically a symmetric pyrimidine or triazine ring, having one or two substituents such as methyl, ethyl, trifluoromethyl, methoxy, ethoxy, methylamino, dimethylamino, ethylamino and the halogens. Sulfonylurea herbicides can be in the form of the free acid or a salt. In the free acid form the sulfonamide nitrogen on the bridge is not deprotonated (i.e., —S(O)2NHC(O)NH(R)—), while in the salt form the sulfonamide nitrogen atom on the bridge is deprotonated (i.e., —S(O)2NC(O)NH(R)—), and a cation is present, typically of an alkali metal or alkaline earth metal, most commonly sodium or potassium. Sulfonylurea compounds include, for example, compound classes such as pyrimidinylsulfonylurea compounds, triazinylsulfonylurea compounds, thiadiazolylurea compounds, and pharmaceuticals such as antidiabetic drugs, as well as salts and other derivatives thereof. Examples of pyrimidinylsulfonylurea compounds include amidosulfuron, azimsulfuron, bensulfuron, bensulfuron-methyl, chlorimuron, chlorimuron-ethyl, cyclosulfamuron, ethoxysulfuron, flazasulfuron, flucetosulfuron, flupyrsulfuron, flupyrsulfuron-methyl, foramsulfuron, halosulfuron, halosulfuron-methyl, imazosulfuron, mesosulfuron, mesosulfuron-methyl, nicosulfuron, orthosulfamuron, oxasulfuron, primisulfuron, primisulfuron-methyl, pyrazosulfuron, pyrazosulfuron-ethyl, rimsulfuron, sulfometuron, sulfometuron-methyl, sulfosulfuron, trifloxysulfuron and salts and derivatives thereof. Examples of triazinylsulfonylurea compounds include chlorsulfuron, cinosulfuron, ethametsulfuron, ethametsulfuron-methyl, iodosulfuron, iodosulfuron-methyl, metsulfuron, metsulfuron-methyl, prosulfuron, thifensulfuron, thifensulfuron-methyl, triasulfuron, tribenuron, tribenuron-methyl, triflusulfuron, triflusulfuron-methyl, tritosulfuron and salts and derivatives thereof. Examples of thiadiazolylurea compounds include buthiuron, ethidimuron, tebuthiuron, thiazafluron, thidiazuron and salts and derivatives thereof. Examples of antidiabetic drugs include acetohexamide, chlorpropamide, tolbutamide, tolazamide, glipizide, gliclazide, glibenclamide (glyburide), gliquidone, glimepiride and salts and derivatives thereof. In some examples the isolated SuR polypeptides specifically bind to more than one sulfonylurea compound. In some examples the sulfonylurea compound is selected from the group consisting of chlorsulfuron, ethametsulfuron-methyl, metsulfuron-methyl, thifensulfuron-methyl, sulfometuron-methyl, tribenuron-methyl, chlorimuron-ethyl, nicosulfuron, and rimsulfuron.

Compositions also include isolated polynucleotides encoding SuR polypeptides that specifically bind to a tetracycline operator, wherein the specific binding is regulated by a sulfonylurea compound. In some examples the isolated polynucleotides encode sulfonylurea repressor (SuR) polypeptides comprising an amino acid substitution in the ligand binding domain of a wild type tetracycline repressor protein. In class B and D wild type TetR proteins, amino acid residues 6-52 represent the DNA binding domain. The remainder of the protein is involved in ligand binding and subsequent allosteric modification. For class B TetR residues 53-207 represent the ligand binding domain, while residues 53-218 comprise the ligand binding domain for the class D TetR. In some examples the isolated polynucleotides encode SuR polypeptides comprising an amino acid substitution in the ligand binding domain of a wild type TetR(B) protein. In some examples the polynucleotides encode SuR polypeptides comprising an amino acid substitution in the ligand binding domain of a wild type TetR(B) protein of SEQ ID NO:1.

In some examples the isolated polynucleotides encode SuR polypeptides comprising an amino acid, or any combination of amino acids, selected from the amino acid diversity shown in FIG. 1, wherein the amino acid residue position corresponds to the equivalent position using the amino acid numbering of a wild type TetR(B) exemplified by SEQ ID NO:1. In some examples the isolated polynucleotides encode SuR polypeptides comprising a ligand binding domain comprising at least 10%, 20%, 30%, 40%, 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the amino acid residues shown in FIG. 1, wherein the amino acid residue position corresponds to the equivalent position using the amino acid numbering of wild type TetR(B). In some examples the isolated polynucleotides encode SuR polypeptides comprising at least 10%, 20%, 30%, 40%, 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of the amino acid residues shown in FIG. 1, wherein the amino acid residue position corresponds to the equivalent position using the amino acid numbering of wild type TetR(B). In some examples the wild type TetR(B) is SEQ ID NO:1.

In some examples the isolated polynucleotides encode SuR polypeptides comprising a ligand binding domain comprising an amino acid substitution at a residue position selected from the group consisting of position 55, 60, 64, 67, 82, 86, 100, 104, 105, 108, 113, 116, 134, 135, 138, 139, 147, 151, 170, 173, 174, 177 and any combination thereof, wherein the amino acid residue position and substitution corresponds to the equivalent position using the amino acid numbering of a wild type TetR(B). In some examples the isolated polynucleotides encode SuR polypeptides further comprising an amino acid substitution at a residue position selected from the group consisting of 109, 112, 117, 131, 137, 140, 164 and any combination thereof. In some examples the wild type TetR(B) polypeptide sequence is SEQ ID NO:1.

In some examples the isolated polynucleotides encode SuR polypeptides having at least about 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the ligand binding domain shown as amino acid residues 53-207 of SEQ ID NO:1, wherein the sequence identity is determined over the full length of the ligand binding domain using a global alignment method. In some examples the global alignment method is GAP, wherein the default parameters are for an amino acid sequence % identity and % similarity using a GAP Weight of 8 and a Length Weight of 2, and the BLOSUM62 scoring matrix.

In some examples the isolated polynucleotides encode SuR polypeptides having at least about 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:1, wherein the sequence identity is determined over the full length of the polypeptide using a global alignment method. In some examples the global alignment method is GAP, wherein the default parameters are for an amino acid sequence % identity and % similarity using a GAP Weight of 8 and a Length Weight of 2, and the BLOSUM62 scoring matrix.

In some examples the isolated polynucleotides include nucleic acid sequences that selectively hybridize under stringent hybridization conditions to a polynucleotide encoding a SuR polypeptide. Polynucleotides that selectively hybridize are polynucleotides which bind to a target sequence at a level of at least 2-fold over background as compared to hybridization to a non-target sequence. Stringent conditions are sequence-dependent and condition-dependent. Typical stringent conditions are those in which the salt concentration about 0.01 to 1.0 M at pH 7.0-8.3 at 30° C. for short probes (e.g., 10 to 50 nucleotides) or about 60° C. for long probes (e.g., greater than 50 nucleotides). Stringent conditions may include formamide or other destabilizing agents. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is impacted by post-hybridization wash conditions, typically via ionic strength and temperature. For DNA-DNA hybrids, the Tₘ can be approximated from the equation of Meinkoth & Wahl (1984) Anal Biochem 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995). In some examples, the isolated polynucleotides encoding SuR polypeptides specifically hybridize to a polynucleotide of SEQ ID NO:420-836 under moderately stringent conditions or under highly stringent conditions.

In some examples the isolated polynucleotide encodes a SuR polypeptide comprising an amino acid sequence that can be optimally aligned with a polypeptide sequence of L7-1A04 (SEQ ID NO:220), L1-22 (SEQ ID NO:7), L1-29 (SEQ ID NO:10), L1-02 (SEQ ID NO:3), L1-07 (SEQ ID NO:4), L1-20 (SEQ ID NO:6), L1-44 (SEQ ID NO:13), L6-3A09 (SEQ ID NO:402), L6-3H02 (SEQ ID NO:94), L7-4E03 (SEQ ID NO:403), L10-84(B12) (SEQ ID NO:404), or L13-46 (SEQ ID NO:405) to generate a BLAST bit score of at least 200, 250, 275, 300, 325, 350, 375, 400, 425, 450, 475, 500, 525, 550, 575, 600, 625, 650, 675, 700, or 750, wherein the BLAST alignment used the BLOSUM62 matrix, a gap existence penalty of 11, and a gap extension penalty of 1. In some examples the isolated polynucleotide encodes a SuR polypeptide comprising an amino acid sequence that can be optimally aligned with a polypeptide sequence of L7-1A04 (SEQ ID NO:220) to generate a BLAST bit score of at least 374, optimally aligned with a polypeptide sequence of L1-22 (SEQ ID NO:7) to generate a BLAST bit score of at least 387, optimally aligned with a polypeptide sequence of L1-29 (SEQ ID NO:10) to generate a BLAST bit score of at least 393, optimally aligned with a polypeptide sequence of L1-07 (SEQ ID NO:4) to generate a BLAST bit score of at least 388, optimally aligned with a polypeptide sequence of L6-3A09 (SEQ ID NO:402) to generate a BLAST bit score of at least 381, optimally aligned with a polypeptide sequence of L7-4E03 (SEQ ID NO:403) to generate a BLAST bit score of at least 368, or optimally aligned with a polypeptide sequence of L13-46 (SEQ ID NO:405) to generate a BLAST bit score of at least 320, wherein the BLAST alignment used the BLOSUM62 matrix, a gap existence penalty of 11, and a gap extension penalty of 1. In some examples the isolated polynucleotide encodes a SuR polypeptides comprising an amino acid sequence that can be optimally aligned with a polypeptide sequence of L7-1A04 (SEQ ID NO:220), L1-22 (SEQ ID NO:7), L1-29 (SEQ ID NO:10), L1-02 (SEQ ID NO:3), L1-07 (SEQ ID NO:4), L1-20 (SEQ ID NO:6), L1-44 (SEQ ID NO:13), L6-3A09 (SEQ ID NO:402), L6-3H02 (SEQ ID NO:94), L7-4E03 (SEQ ID NO:403), L10-84(B12) (SEQ ID NO:404), or L13-46 (SEQ ID NO:405) to generate a percent sequence identity of at least 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity, wherein the sequence identity is determined by BLAST alignment using the BLOSUM62 matrix, a gap existence penalty of 11, and a gap extension penalty of 1. In some examples the isolated polynucleotide encodes a SuR polypeptide comprising an amino acid sequence that can be optimally aligned with a polypeptide sequence of L7-1A04 (SEQ ID NO:220) to generate a percent sequence identity of at least 88% sequence identity, optimally aligned with a polypeptide sequence of L1-22 (SEQ ID NO:7) to generate a percent sequence identity of at least 92% sequence identity, optimally aligned with a polypeptide sequence of L1-07 (SEQ ID NO:4) to generate a percent sequence identity of at least 93% sequence identity, optimally aligned with a polypeptide sequence of L1-20 (SEQ ID NO:6) to generate a percent sequence identity of at least 93% sequence identity, optimally aligned with a polypeptide sequence of L1-44 (SEQ ID NO:13) to generate a percent sequence identity of at least 93% sequence identity, optimally aligned with a polypeptide sequence of L6-3H02 (SEQ ID NO:94) to generate a percent sequence identity of at least 90% sequence identity, optimally aligned with a polypeptide sequence of L10-84(B12) (SEQ ID NO:404) to generate a percent sequence identity of at least 86% sequence identity, or optimally aligned with a polypeptide sequence of L13-46 (SEQ ID NO:405) to generate a percent sequence identity of at least 86% sequence identity, wherein the sequence identity is determined by BLAST alignment using the BLOSUM62 matrix, a gap existence penalty of 11, and a gap extension penalty of 1. In some examples the percent identity is determined using a global alignment method using the GAP algorithm with default parameters for an amino acid sequence % identity and % similarity using GAP Weight of 8 and Length Weight of 2, and the BLOSUM62 scoring matrix. In some examples the isolated polynucleotide encodes a SuR polypeptide comprising an amino acid sequence that can be optimally aligned with a polypeptide sequence of L7-1A04 (SEQ ID NO:220), L1-22 (SEQ ID NO:7), L1-29 (SEQ ID NO:10), L1-02 (SEQ ID NO:3), L1-07 (SEQ ID NO:4), L1-20 (SEQ ID NO:6), L1-44 (SEQ ID NO:13), L6-3A09 (SEQ ID NO:402), L6-3H02 (SEQ ID NO:94), L7-4E03 (SEQ ID NO:403), L10-84(B12) (SEQ ID NO:404), or L13-46 (SEQ ID NO:405) to generate a BLAST similarity score of at least 400, 425, 450, 475, 500, 525, 550, 575, 600, 625, 650, 675, 600, 750, 800, 850, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, or 1200, wherein BLAST alignment used the BLOSUM62 matrix, a gap existence penalty of 11, and a gap extension penalty of 1. In some examples the isolated polynucleotide encodes a SuR polypeptide comprising an amino acid sequence that can be optimally aligned with a polypeptide sequence of L1-29 (SEQ ID NO:10) to generate a BLAST similarity score of at least 1006, optimally aligned with a polypeptide sequence of L1-07 (SEQ ID NO:4) to generate a BLAST similarity score of at least 996, optimally aligned with a polypeptide sequence of L6-3A09 (SEQ ID NO:402) to generate a BLAST similarity score of at least 978, optimally aligned with a polypeptide sequence of L7-4E03 (SEQ ID NO:403) to generate a BLAST similarity score of at least 945, or optimally aligned with a polypeptide sequence of L13-46 (SEQ ID NO:405) to generate a BLAST similarity score of at least 819, wherein the BLAST alignment used the BLOSUM62 matrix, a gap existence penalty of 11, and a gap extension penalty of 1. In some examples the isolated polynucleotide encodes a SUR polypeptide comprising an amino acid sequence that can be optimally aligned with a polypeptide sequence of L7-1A04 (SEQ ID NO:220), L1-22 (SEQ ID NO:7), L1-29 (SEQ ID NO:10), L1-02 (SEQ ID NO:3), L1-07 (SEQ ID NO:4), L1-20 (SEQ ID NO:6), L1-44 (SEQ ID NO:13), L6-3A09 (SEQ ID NO:402), L6-3H02 (SEQ ID NO:94), L7-4E03 (SEQ ID NO:403), L10-84(B12) (SEQ ID NO:404), or L13-46 (SEQ ID NO:405) to generate a BLAST e-value score of at least e-60, e-70, e-80, e-85, e-90, e-95, e-100, e-105, e-106, e-107, e-108, e-109, e-110, e-111, e-112, e-113, e-114, e-115, e-116, e-117, e-118, e-119, e-120, or e-125, wherein BLAST alignment used the BLOSUM62 matrix, a gap existence penalty of 11, and a gap extension penalty of 1. In some examples the isolated polynucleotide encodes a SuR polypeptide comprising SuR polypeptides comprise an amino acid sequence that can be optimally aligned with a polypeptide sequence of L1-02 (SEQ ID NO:3) to generate a BLAST e-value score of at least e-112, optimally aligned with a polypeptide sequence of L1-07 (SEQ ID NO:4) to generate a BLAST e-value score of at least e-111, optimally aligned with a polypeptide sequence of L1-20 (SEQ ID NO:6) to generate a BLAST e-value score of at least e-111, optimally aligned with a polypeptide sequence of L6-3A09 (SEQ ID NO:402) to generate a BLAST e-value score of at least e-108, optimally aligned with a polypeptide sequence of L7-4E03 (SEQ ID NO:403) to generate a BLAST e-value score of at least e-105 , or optimally aligned with a polypeptide sequence of L13-46 (SEQ ID NO:405) to generate a BLAST e-value score of at least e-90 ,wherein the BLAST alignment used the BLOSUM62 matrix, a gap existence penalty of 11, and a gap extension penalty of 1. In some examples the isolated polynucleotide encodes a polypeptide selected from the group consisting of SEQ ID NO:3-419. In some examples the isolated polynucleotide comprises a polynucleotide sequence of SEQ ID NO:420-836, or the complementary polynucleotide thereof.

In some examples the isolated polynucleotide encodes a SuR polypeptide comprising a ligand binding domain from a polypeptide selected from the group consisting of SEQ ID NO:3-419. In some examples the isolated polynucleotide encodes a SuR polypeptide comprising an amino acid sequence selected from the group consisting of SEQ ID NO:3-419. In some examples the encoded SuR polypeptide is selected from the group consisting of SEQ ID NO:3-419, and the sulfonylurea compound is selected from the group consisting of chlorsulfuron, ethametsulfuron-methyl, metsulfuron-methyl, sulfometuron-methyl, and thifensulfuron-methyl. In some examples the isolated polynucleotide comprises a polynucleotide sequence of SEQ ID NO:420-836, or the complementary polynucleotide thereof.

In some examples the isolated SuR polynucleotide encodes a SuR polypeptide having an equilibrium binding constant for a sulfonylurea compound greater than 0.1 nM and less than 10 μM. In some examples the encoded SuR polypeptide has an equilibrium binding constant for a sulfonylurea compound of at least 0.1 nM, 0.5 nM, 1 nM, 10 nM, 50 nM, 100 nM, 250 nM, 500 nM, 750 nM, 1 μM, 5 μM, 7 μM but less than 10 μM. In some examples the encoded SuR polypeptide has an equilibrium binding constant for a sulfonylurea compound of at least 0.1 nM, 0.5 nM, 1 nM, 10 nM, 50 nM, 100 nM, 250 nM, 500 nM, 750 nM but less than 1 μM. In some examples the encoded SuR polypeptide has an equilibrium binding constant for a sulfonylurea compound greater than 0 nM, but less than 0.1 nM, 0.5nM, 1 nM, 10 nM, 50 nM, 100 nM, 250 nM, 500 nM, 750 nM, 1 μM, 5 μM, 7 μM, or 10 μM. In some examples the sulfonylurea compound is a chlorsulfuron, an ethametsulfuron, a metsulfuron, a sulfometuron, and/or a thifensulfuron compound.

In some examples the isolated SuR polynucleotide encodes a SuR polypeptide having an equilibrium binding constant for an operator sequence greater than 0.1 nM and less than 10 μM. In some examples the encoded SuR polypeptide has an equilibrium binding constant for an operator sequence of at least 0.1 nM, 0.5 nM, 1 nM, 10 nM, 50 nM, 100 nM, 250 nM, 500 nM, 750 nM, 1 μM, 5 μM, 7 μM but less than 10 μM. In some examples the encoded SuR polypeptide has an equilibrium binding constant for an operator sequence of at least 0.1 nM, 0.5 nM, 1 nM, 10 nM, 50 nM, 100 nM, 250 nM, 500 nM, 750 nM but less than 1 μM. In some examples the encoded SuR polypeptide has an equilibrium binding constant for an operator sequence greater than 0 nM, but less than 0.1 nM, 0.5 nM, 1 nM, 10 nM, 50 nM, 100 nM, 250 nM, 500 nM, 750 nM, 1 μM, 5 μM, 7 μM or 10 μM. In some examples the operator sequence is a Tet operator sequence. In some examples the Tet operator sequence is a TetR(A) operator sequence, a TetR(B) operator sequence, a TetR(D) operator sequence, TetR(E) operator sequence, a TetR(H) operator sequence or a functional derivative thereof. In some examples the isolated polynucleotides encoding SuR polypeptides, recombinase, or a trait of interest comprise codon composition profiles representative of codon preferences for particular host cells, or host cell organelles. In some examples the isolated polynucleotides comprise prokaryote preferred codons. In some examples the isolated polynucleotides comprise bacteria preferred codons. In some examples the bacteria is E. coli or Agrobacterium. In some examples the isolated polynucleotides comprise plastid preferred codons. In some examples the isolated polynucleotides comprise eukaryote preferred codons. In some examples the isolated polynucleotides comprise nuclear preferred codons. In some examples the isolated polynucleotides comprise plant preferred codons. In some examples the isolated polynucleotides comprise monocotyledonous plant preferred codons. In some examples the isolated polynucleotides comprise corn, rice, sorghum, barley, wheat, rye, switch grass, sugarcane, turf grass and/or oat preferred codons. In some examples the isolated polynucleotides comprise dicotyledonous plant preferred codons. In some examples the isolated polynucleotides comprise soybean, sunflower, safflower, Brassica, alfalfa, Arabidopsis, tobacco and/or cotton preferred codons. In some examples the isolated polynucleotides comprise yeast preferred codons. In some examples the isolated polynucleotides comprise mammalian preferred codons. In some examples the isolated polynucleotides comprise insect preferred codons.

Compositions also include isolated polynucleotides fully complementary to a polynucleotide encoding a SuR polypeptide, expression cassettes, replicons, vectors, T-DNAs, DNA libraries, host cells, tissues and/or organisms comprising the polynucleotides encoding the SuR polypeptides and/or complements or derivatives thereof. In some examples the polynucleotide is stably incorporated into a genome of the host cell, tissue and/or organism. In some examples the host cell is a prokaryote, including E. coli and Agrobacterium strains. In some examples the host is a eukaryote, including for example yeast, insects, plants and mammals.

Repressible promoters comprising at least one operator sequence are also provided. Expression from these promoters is controlled by a repressor that binds to the operator sequence, wherein binding of the repressor to the operator is regulated by the presence or absence of chemical ligand. In some examples, the repressible promoter comprises at least one tet operator sequence. Repressors include tet repressors and sulfonylurea-regulated repressors. Binding of a tet repressor to a tet operator is regulated by tetracycline compounds and analogs thereof. Binding of a sulfonylurea-responsive repressor to a tet operator is controlled by sulfonylurea compounds and analogs thereof. In some examples, the repressible promoter comprises a tet operator sequence located within 0-30 nucleotides 5′ or 3′ of the TATA box. In some examples, the tet operator sequence is located within 20, 19, 18, 17, 16, 15, 14, 13, 12, 11, 10, 9, 8, 7, 6, 5, 4, 3, 2, 1, or 0 nt of the TATA box. In some examples the tet operator sequence may partially overlap with the TATA box sequence. In some examples the tet operator sequence is SEQ ID NO:848. In some examples the promoter is active in plant cells. In some examples the promoter is a constitutive promoter. In other examples the promoter is a non-constitutive promoter. In some examples the non-constitutive promoter is a tissue-preferred promoter. In some examples the tissue-preferred promoter is primarily expressed in roots, leaves, stems, flowers, silks, anthers, pollen, meristem, seed, endosperm, or embryos. In some examples the promoter is a plant actin promoter, a banana streak virus promoter (BSV), an MMV promoter, an enhanced MMV promoter (dMMV), a plant P450 promoter, or an elongation factor 1a (EF1A) promoter. In some examples the promoter is a plant actin promoter (SEQ ID NO:849), a banana streak virus promoter (BSV) (SEQ ID NO:850), a mirabilis mosaic virus promoter (MMV) (SEQ ID NO:851), an enhanced MMV promoter (dMMV) (SEQ ID NO:852), a plant P450 promoter (MP1) (SEQ ID NO:853), or an elongation factor 1a (EF1A) promoter (SEQ ID NO:854). In some examples, the repressible promoter comprises two tet operator sequences, wherein the 1^{st} tet operator sequence is located within 0-30 nt 5′ of the TATA box, and the 2^{nd} tet operator sequence is located within 0-30 nt 3′ of the TATA box. In some examples, the first and/or the second tet operator sequence is located within 20, 19, 18, 17, 16, 15, 14, 13, 12, 11, 10, 9, 8, 7, 6, 5, 4, 3, 2, 1, or 0 nt of the TATA box. In some examples the first and/or the second tet operator sequence may partially overlap with the TATA box sequence. In some examples the first and/or the second tet operator sequence is SEQ ID NO:848. In some examples, the repressible promoter comprises three tet operator sequences, wherein the 1^{st} tet operator sequence is located within 0-30 nt 5′ of the TATA box, and the 2^{nd} tet operator sequence is located within 0-30 nt 3′ of the TATA box, and the 3^{rd} tet operator is located with 0-50 nt of the transcriptional start site (TSS). In some examples, the 1^{st} and/or the 2^{nd} tet operator sequence is located within 20, 19, 18, 17, 16, 15, 14, 13, 12, 11, 10, 9, 8, 7, 6, 5, 4, 3, 2, 1, or 0 nt of the TATA box. In some examples, the 3^{rd} tet operator sequence is located within 50, 45, 40, 35, 30, 25, 20, 19, 18, 17, 16, 15, 14, 13, 12, 11, 10, 9, 8, 7, 6, 5, 4, 3, 2, 1, or 0 nt of the TSS. In some examples the 3^{rd} tet operator is located 5′ of the TSS. In some examples the 3^{rd} tet operator sequence may partially overlap with the TSS sequence. In some examples the 1^{st}, 2^{nd} and/or the 3^{rd} tet operator sequence is SEQ ID NO:848. In some examples the promoter is a plant actin promoter (actin/Op) (SEQ ID NO:855), a banana streak virus promoter (BSV/Op) (SEQ ID NO:856), a mirabilis mosaic virus promoter (MMV/Op) (SEQ ID NO:857), an enhanced MMV promoter (dMMV/Op) (SEQ ID NO:858), a plant P450 promoter (MP1/Op) (SEQ ID NO:859), or an elongation factor 1a (EF1A/Op) promoter (SEQ ID NO:860). In some examples, the promoter comprises a polynucleotide sequence having at least about 50%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:885, 856, 857, 858, 859, or 860, wherein the promoter retains repressible promoter activity. In a specific example, the promoter comprises a polynucleotide sequence having at least 95% sequence identity to SEQ ID NO:885, 856, 857, 858, 859, or 860, wherein the promoter retains repressible promoter activity. A promoter with “repressible promoter activity” will direct expression of an operably linked polynucleotide, wherein its ability to direct transcription depends on the presence or absence of a chemical ligand (i.e., a tetracycline compound, a sulfonylurea compound) and a repressor protein.

Methods using the gene switch compositions and/or elements thereof are further provided. In one example, methods of regulating transcription of a polynucleotide of interest in a host cell are provided, the methods comprising: providing a cell comprising the polynucleotide of interest operably linked to a repressible promoter comprising at least one tetracycline operator sequence; providing a SuR polypeptide and, providing a sulfonylurea compound, thereby regulating transcription of the polynucleotide of interest. Any host cell can be used, including for example prokaryotic cells such as bacteria, and eukaryotic cells, including yeast, plant, insect, and mammalian cells. In some examples providing the SuR polypeptide comprises contacting the cell with an expression cassette comprising a promoter functional in the cell operably linked to a polynucleotide that encodes the SuR polypeptide. In some examples the methods are used to activate expression of a polynucleotide of interest. In some examples expression of the polynucleotide of interest is activated in various tissues or cells, restricted to selected tissue or cell type, restricted to specific developmental stage(s), restricted to specific environmental conditions, and/or restricted to specific generation of a plant or progeny thereof. In some examples the polynucleotide of interest is primarily expressed in roots, leaves, stems, flowers, silks, anthers, pollen, meristem, germline, seed, endosperm, embryos, or progeny. In some examples expression of the polynucleotide of interest occurs primarily at specific times, which include but are not limited to seed or plant developmental stages, vegetative growth, reproductive cycle, response to environmental conditions, response to pest or pathogen presence, response to chemical compounds, or any combination thereof. In some examples expression of the polynucleotide of interest is reduced, inhibited, or blocked in various tissues or cells, which may be restricted to selected tissue or cell type, restricted to specific developmental stage(s), restricted to specific environmental conditions, and/or restricted to specific generation of a plant or progeny thereof. In some examples expression of the polynucleotide of interest is primarily inhibited in roots, leaves, stems, flowers, silks, anthers, pollen, meristem, germline, seed, endosperm, embryos, or progeny. In some examples expression of the polynucleotide of interest occurs primarily inhibited at specific times, which include but are not limited to seed or plant developmental stages, vegetative growth, reproductive cycle, response to environmental conditions, response to pest or pathogen presence, response to chemical compounds, or any combination thereof.

In another example, methods of regulating transcription of a polynucleotide of interest in a host cell are provided, the methods comprising: providing a cell comprising the polynucleotide of interest operably linked to a repressible promoter comprising at least one tetracycline operator sequence; providing a TetR polypeptide and, providing a tetracycline compound, thereby regulating transcription of the polynucleotide of interest. In some examples, the repressible promoter is a plant actin promoter, a banana streak virus promoter (BSV), an MMV promoter, an enhanced MMV promoter (dMMV), a plant P450 promoter, or an elongation factor 1a (EF1A) promoter. In some examples the promoter is a plant actin promoter (actin/Op) (SEQ ID NO:855), a banana streak virus promoter (BSV/Op) (SEQ ID NO:856), a mirabilis mosaic virus promoter (MMV/Op) (SEQ ID NO:857), an enhanced MMV promoter (dMMV/Op) (SEQ ID NO:858), a plant P450 promoter (MP1/Op) (SEQ ID NO:859), or an elongation factor 1a (EF1A/Op) promoter (SEQ ID NO:860). In some examples, the promoter comprises a polynucleotide sequence having at least about 50%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:885, 856, 857, 858, 859, or 860, wherein the promoter retains repressible promoter activity. In a specific example, the promoter comprises a polynucleotide sequence having at least 95% sequence identity to SEQ ID NO:885, 856, 857, 858, 859, or 860, wherein the promoter retains repressible promoter activity.

Any host cell can be used, including for example prokaryotic cells such as bacteria, and eukaryotic cells, including yeast, plant, insect, and mammalian cells. In some examples providing the TetR polypeptide comprises contacting the cell with an expression cassette comprising a promoter functional in the cell operably linked to a polynucleotide that encodes the TetR polypeptide. In some examples the methods are used to activate expression of a polynucleotide of interest. In some examples expression of the polynucleotide of interest is activated in various tissues or cells, restricted to selected tissue or cell type, restricted to specific developmental stage(s), restricted to specific environmental conditions, and/or restricted to specific generation of a plant or progeny thereof. In some examples the polynucleotide of interest is primarily expressed in roots, leaves, stems, flowers, silks, anthers, pollen, meristem, germline, seed, endosperm, embryos, or progeny. In some examples expression of the polynucleotide of interest occurs primarily at specific times, which include but are not limited to seed or plant developmental stages, vegetative growth, reproductive cycle, response to environmental conditions, response to pest or pathogen presence, response to chemical compounds, or any combination thereof. In some examples expression of the polynucleotide of interest is reduced, inhibited, or blocked in various tissues or cells, which may be restricted to selected tissue or cell type, restricted to specific developmental stage(s), restricted to specific environmental conditions, and/or restricted to specific generation of a plant or progeny thereof. In some examples expression of the polynucleotide of interest is primarily inhibited in roots, leaves, stems, flowers, silks, anthers, pollen, meristem, germline, seed, endosperm, embryos, or progeny. In some examples expression of the polynucleotide of interest occurs primarily inhibited at specific times, which include but are not limited to seed or plant developmental stages, vegetative growth, reproductive cycle, response to environmental conditions, response to pest or pathogen presence, response to chemical compounds, or any combination thereof.

Methods include stringently and/or specifically controlling expression of a polynucleotide of interest. Stringency and/or specificity modulated by selecting the combination of elements used in the switch. These include, but are not limited to the promoter operably linked to the repressor, the repressor, the repressible promoter operably linked to the polynucleotide of interest, and optionally the polynucleotide of interest. Further control is provided by selection, dosage, conditions, and/or timing of the application of the chemical ligand. In some examples the expression of the polynucleotide of interest can be controlled more stringently, controlled in various tissues or cells, restricted to selected tissue or cell type, restricted to specific developmental stage(s), restricted to specific environmental conditions, and/or restricted to specific generation of a plant or progeny thereof. In some examples the repressor is operably linked to a constitutive promoter. In some examples the repressor is operably linked to a non-constitutive promoter, including but not limited to a tissue preferred promoter, an inducible promoter, a repressible promoter, a developmental stage preferred promoter, or a promoter having more than one of these properties. In some examples expression of the polynucleotide of interest is primarily regulated in roots, leaves, stems, flowers, silks, anthers, pollen, meristem, germline, seed, endosperm, embryos, or progeny. These methods provide means to alter the phenotype and/or genotype of a cell, tissue, plant, and/or seed. An altered genotype includes any heritable modification to any sequence in a plant genome. An altered phenotype includes any scenario wherein a cell, tissue, plant, and/or seed exhibits a characteristic or trait that distinguishes it from its unaltered state. Altered phenotypes included but are not limited to a different growth habit, altered flower color, altered relative maturity, altered yield, altered fertility, altered flowering time, altered disease tolerance, altered insect tolerance, altered herbicide tolerance, altered stress tolerance, altered water tolerance, altered drought tolerance, altered seed characteristics, altered morphology, altered agronomic characteristic, altered metabolism, altered gene expression profile, altered ploidy, altered crop quality, altered forage quality, altered silage quality, altered processing characteristics, and the like.

In some examples, the methods use a gene switch which may comprise additional elements. In some examples, one or more additional elements may provide means by which expression of the polynucleotide of interest can be controlled more stringently, controlled in various tissues or cells, restricted to selected tissue or cell type, restricted to specific developmental stage(s), restricted to specific environmental conditions, and/or restricted to specific generation of a plant or progeny thereof. In some examples those elements include site-specific recombination sites, site-specific recombinases, or combinations thereof.

In some methods, the gene switch may comprise a polynucleotide encoding a repressor, a promoter linked to a polynucleotide of interest, a sequence flanked by site-specific recombination sites, and a repressible promoter operably linked to a site-specific recombinase that specifically recognizes the site-specific recombination sites and implements a recombination event. In some examples, the recombination event is excision of the sequence flanked by the recombination sites. In some instances, the excision creates an operable linkage between the promoter and the polynucleotide of interest. In some examples, the promoter operably linked to the polynucleotide of interest is a non-constitutive promoter, including but not limited to a tissue preferred promoter, an inducible promoter, a repressible promoter, a developmental stage preferred promoter, or a promoter having more than one of these properties. In some examples expression of the polynucleotide of interest is primarily regulated in roots, leaves, stems, flowers, silks, anthers, pollen, meristem, germline, seed, endosperm, embryos, or progeny.

In other methods, the gene switch may comprise a polynucleotide encoding a repressor, a repressible promoter linked to a polynucleotide of interest, a sequence flanked by site-specific recombination sites, and a site-specific recombinase that specifically recognizes the site-specific recombination sites and implements a recombination event. In some examples, the recombination event is excision of the sequence flanked by the recombination sites. In some instances, the excision creates an operable linkage between the repressible promoter and the polynucleotide of interest. In some examples, the sequence flanked by recombination sites comprises a recombinase expression cassette. In some examples expression of the polynucleotide of interest is primarily regulated in roots, leaves, stems, flowers, silks, anthers, pollen, meristem, germline, seed, endosperm, embryos, or progeny. In some examples the excision occurs in a parent such that the progeny inherit the post-excision product.

In some examples, the gene switch may comprise a polynucleotide encoding a repressor, a promoter operably linked to a polynucleotide of interest flanked by site-specific recombination sites, and a repressible promoter operably linked to a site-specific recombinase that specifically recognizes the site-specific recombination sites and implements a recombination event. In some examples, the recombination event is excision of the sequence flanked by the recombination sites. In some examples, the promoter operably linked to the polynucleotide of interest is a non-constitutive promoter, including but not limited to a tissue preferred promoter, an inducible promoter, a repressible promoter, a developmental stage preferred promoter, or a promoter having more than one of these properties. In some examples expression of the polynucleotide of interest is primarily regulated in roots, leaves, stems, flowers, silks, anthers, pollen, meristem, germline, seed, endosperm, embryos, or progeny.

In another example, the methods comprise providing a gene switch comprising a polynucleotide encoding a repressor, a promoter linked to a polynucleotide of interest, a sequence flanked by site-specific recombination sites, and a repressible promoter operably linked to a site-specific recombinase that specifically recognizes the site-specific recombination sites and implements a recombination event. In some examples, the recombination event is inversion of the sequence flanked by the recombination sites. In some instances, the inversion creates an operable linkage between the promoter and the polynucleotide of interest. In some examples, the promoter operably linked to the polynucleotide of interest is a non-constitutive promoter, including but not limited to a tissue preferred promoter, an inducible promoter, a repressible promoter, a developmental stage preferred promoter, or a promoter having more than one of these properties. In some examples expression of the polynucleotide of interest is primarily regulated in roots, leaves, stems, flowers, silks, anthers, pollen, meristem, germline, seed, endosperm, or embryos. In some examples the inversion occurs in a parent such that the progeny inherit the post-inversion product.

In other methods, the provided gene switch may comprise a polynucleotide encoding a repressor, a repressible promoter linked to a polynucleotide of interest, a sequence flanked by site-specific recombination sites, and a site-specific recombinase that specifically recognizes the site-specific recombination sites and implements a recombination event. In some examples, the recombination event is inversion of the sequence flanked by recombination sites. In some instances, the inversion creates an operable linkage between the repressible promoter and the polynucleotide of interest. In some cases, the sequence flanked by site-specific recombination sites is the polynucleotide of interest. In some cases, the sequence flanked by site-specific recombination sites is the repressible promoter. In some examples, a recombinase expression cassette is provided, wherein the recombinase is operably linked to a non-constitutive promoter, including but not limited to a tissue preferred promoter, an inducible promoter, a repressible promoter, a developmental stage preferred promoter, or a promoter having more than one of these properties. In some examples expression of the polynucleotide of interest is primarily expressed in roots, leaves, stems, flowers, silks, anthers, pollen, meristem, seed, endosperm, embryos, or progeny. In some examples the inversion occurs in a parent such that the progeny inherit the post-inversion product.

In other examples, methods for altering a genotype or phenotype are provided. In some examples the methods comprise providing a cell comprising the polynucleotide of interest operably linked to a repressible promoter comprising at least one tetracycline operator sequence; providing a SuR polypeptide and, providing a sulfonylurea compound, thereby altering a genotype and/or phenotype of the cell. Any host cell can be used, including for example prokaryotic cells such as bacteria, and eukaryotic cells, including yeast, plant, insect, and mammalian cells. In some examples providing the SuR polypeptide comprises contacting the cell with an expression cassette comprising a promoter functional in the cell operably linked to a polynucleotide that encodes the SuR polypeptide. In some examples the methods are used to activate expression of a polynucleotide of interest. In some examples expression of the polynucleotide of interest is activated in various tissues or cells, restricted to selected tissue or cell type, restricted to specific developmental stage(s), restricted to specific environmental conditions, and/or restricted to specific generation of a plant or progeny thereof. In some examples the polynucleotide of interest is primarily expressed in roots, leaves, stems, flowers, silks, anthers, pollen, meristem, germline, seed, endosperm, embryos, or progeny. In some examples expression of the polynucleotide of interest occurs primarily at specific times, which include but are not limited to seed or plant developmental stages, vegetative growth, reproductive cycle, response to environmental conditions, response to pest or pathogen presence, response to chemical compounds, or any combination thereof. In some examples expression of the polynucleotide of interest is reduced, inhibited, or blocked in various tissues or cells, which may be restricted to selected tissue or cell type, restricted to specific developmental stage(s), restricted to specific environmental conditions, and/or restricted to specific generation of a plant or progeny thereof. In some examples expression of the polynucleotide of interest is primarily inhibited in roots, leaves, stems, flowers, silks, anthers, pollen, meristem, germline, seed, endosperm, embryos, or progeny. In some examples expression of the polynucleotide of interest occurs primarily inhibited at specific times, which include but are not limited to seed or plant developmental stages, vegetative growth, reproductive cycle, response to environmental conditions, response to pest or pathogen presence, response to chemical compounds, or any combination thereof.

In another example, methods of altering a genotype or phenotype in a host cell are provided, the methods comprising: providing a cell comprising the polynucleotide of interest operably linked to a repressible promoter comprising at least one tetracycline operator sequence; providing a TetR polypeptide and, providing a tetracycline compound, thereby regulating transcription of the polynucleotide of interest. In some examples, the repressible promoter is a plant actin promoter, a banana streak virus promoter (BSV), an MMV promoter, an enhanced MMV promoter (dMMV), a plant P450 promoter, or an elongation factor 1a (EF1A) promoter. In some examples the promoter is a plant actin promoter (actin/Op) (SEQ ID NO:855), a banana streak virus promoter (BSV/Op) (SEQ ID NO:856), a mirabilis mosaic virus promoter (MMV/Op) (SEQ ID NO:857), an enhanced MMV promoter (dMMV/Op) (SEQ ID NO:858), a plant P450 promoter (MP1/Op) (SEQ ID NO:859), or an elongation factor 1a (EF1A/Op) promoter (SEQ ID NO:860). In some examples, the promoter comprises a polynucleotide sequence having at least about 50%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:885, 856, 857, 858, 859, or 860, wherein the promoter retains repressible promoter activity. In a specific example, the promoter comprises a polynucleotide sequence having at least 95% sequence identity to SEQ ID NO:885, 856, 857, 858, 859, or 860, wherein the promoter retains repressible promoter activity.

Any host cell can be used, including for example prokaryotic cells such as bacteria, and eukaryotic cells, including yeast, plant, insect, and mammalian cells. In some examples providing the TetR polypeptide comprises contacting the cell with an expression cassette comprising a promoter functional in the cell operably linked to a polynucleotide that encodes the TetR polypeptide. In some examples the methods are used to activate expression of a polynucleotide of interest. In some examples expression of the polynucleotide of interest is activated in various tissues or cells, restricted to selected tissue or cell type, restricted to specific developmental stage(s), restricted to specific environmental conditions, and/or restricted to specific generation of a plant or progeny thereof. In some examples the polynucleotide of interest is primarily expressed in roots, leaves, stems, flowers, silks, anthers, pollen, meristem, germline, seed, endosperm, embryos, or progeny. In some examples expression of the polynucleotide of interest occurs primarily at specific times, which include but are not limited to seed or plant developmental stages, vegetative growth, reproductive cycle, response to environmental conditions, response to pest or pathogen presence, response to chemical compounds, or any combination thereof. In some examples expression of the polynucleotide of interest is reduced, inhibited, or blocked in various tissues or cells, which may be restricted to selected tissue or cell type, restricted to specific developmental stage(s), restricted to specific environmental conditions, and/or restricted to specific generation of a plant or progeny thereof. In some examples expression of the polynucleotide of interest is primarily inhibited in roots, leaves, stems, flowers, silks, anthers, pollen, meristem, germline, seed, endosperm, embryos, or progeny. In some examples expression of the polynucleotide of interest occurs primarily inhibited at specific times, which include but are not limited to seed or plant developmental stages, vegetative growth, reproductive cycle, response to environmental conditions, response to pest or pathogen presence, response to chemical compounds, or any combination thereof.

In some examples, the sulfonylurea compound is a pyrimidinylsulfonylurea compound (e.g., amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulftiron, pyrazosulfuron, rimsulfuron, sulfometuron, sulfosulfuron and trifloxysulfuron); a triazinylsulfonylurea compound (e.g., chlorsulfuron, cinosulfuron, ethametsulfuron, iodosulfuron, metsulfuron, prosulfuron, thifensulfuron, triasulfuron, tribenuron, triflusulfuron and tritosulfuron); or a thiadazolylurea compound (e.g., cloransulam, diclosulam, florasulam, flumetsulam, metosulam, and penoxsulam). For example, the sulfonylurea compound can be a chlorosulfuron, an ethametsulfuron, a thifensulfuron, a metsulfuron, a sulfometuron, a tribenuron, a chlorimuron, a nicosulfuron, or a rimsulfuron.

In some examples the sulfonylurea compound is an ethametsulfuron. In some examples the ethametsulfuron is provided at a concentration of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 100, 200 or 500 pg/ml. In some examples the SuR polypeptide has a ligand binding domain having at least 50 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to a SuR polypeptide of SEQ ID NO:205-419, wherein the sequence identity is determined over the full length of the polypeptide using a global alignment method. In some examples the global alignment method is GAP, wherein the default parameters are for an amino acid sequence % identity and % similarity using a GAP Weight of 8 and a Length Weight of 2, and the BLOSUM62 scoring matrix. In some examples the polypeptide has a ligand binding domain from a SuR polypeptide selected from the group consisting of SEQ ID NO:205-419. In some examples the polypeptide is selected from the group consisting of SEQ ID NO:205-419. In some examples the polypeptide is encoded by a polynucleotide of SEQ ID NO:622-836.

In some examples the sulfonylurea compound is chlorsulfuron. In some examples the chlorsulfuron is provided at a concentration of about 0.01, 0.02, 0.03, 0.04, 0.05, 0.06, 0.07, 0.08, 0.09, 0.10, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 100, 200 or 500 pg/ml. In some examples the SuR polypeptide has a ligand binding domain having at least 50% 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to a SuR polypeptide of SEQ ID NO:14-204, wherein the sequence identity is determined over the full length of the polypeptide using a global alignment method. In some examples the global alignment method is GAP, wherein the default parameters are for an amino acid sequence % identity and % similarity using GAP Weight of 8 and Length Weight of 2 and the BLOSUM62 scoring matrix. In some examples the polypeptide has a ligand binding domain from a SuR polypeptide selected from the group consisting of SEQ ID NO:14-204. In some examples the polypeptide is selected from the group consisting of SEQ ID NO:14-204. In some examples the polypeptide is encoded by a polynucleotide of SEQ ID NO:431-621.

The ability to tightly regulate gene expression provides means for controlling engineered trait expression and distribution. Such systems may prevent transgene flow into non-transgenic crops or other plants. Chemically-regulated gene switches, gene switch components, and methods of use are provided. Tetracycline repressor was converted to specifically recognize sulfonylurea compounds using protein modeling, DNA shuffling, and screening. For agricultural applications, sulfonylurea compounds are phloem mobile and commercially available, thereby providing a good basis for use as switch ligand chemistry. Following several rounds of modeling and DNA shuffling, repressors that specifically recognize SU chemistry nearly as well as wild type TetR recognizes cognate inducers have been generated. These polypeptides comprise true sulfonylurea repressors (SuRs), which have been validated in planta to demonstrate functionality of the SuR switch system. While exemplified in an agricultural context, these methods and compositions can be used in a wide variety of other settings and organisms.

In general, a gene switch system wherein the chemical used penetrates rapidly and is perceived by all cell types in the organism, but does not perturb any endogenous regulatory networks will be most useful. Other characteristics include the behavior of the sensor component, for example the stringency of regulation and response in the absence or presence of inducer. In general a switch system having tight regulation of the “off” state in the absence of inducer and rapid and intense response in the presence of inducer is preferred.

Expression of the Tn10-operon is regulated by binding of the tet repressor to its operator sequences (Beck et al. (1982) J Bacteriol 150:633-642; Wray & Reznikoff (1983) J Bacteriol 156:1188-1191). The high specificity of tetracycline repressor for the tet operator, the high efficiency of induction by tetracycline and its derivatives, the low toxicity of the inducer, as well as the ability of tetracycline to easily permeate most cells, are the basis for the application of the tet system in somatic gene regulation in eukaryotic cells from animals (Wirtz & Clayton (1995) Science 268:1179-1183; Gossen et al. (1995) Science 268:1766-1769), humans (Deuschle et al. (1995) Mol Cell Biol 15:1907-1914; Furth et al. (1994) PNAS 91:9302-9306; Gossen & Bujard (1992) PNAS 89:5547-5551; Gossen et al. (1995) Science 268:1766-1769) and plant cell cultures (Wilde et al. (1992) EMBO J 11:1251-1259; Gatz et al. (1992) Plant J 2:397-404; Roder et al. (1994) Mol Gen Genet 243:32-28; Ulmasov et al. (1997) Plant Mol Biol 35:417-424).

A number of variations of tetracycline operator/repressor systems have been devised. For example, one system based on conversion of the tet repressor to an activator was developed via fusion of the repressor to a transcriptional transactivation domain such as herpes simplex virus VP16 and the tet repressor (tTA, Gossen & Bujard (1992) PNAS 89:5547-5551). In this system, a minimal promoter is activated in the absence of tetracycline by binding of tTA to tet operator sequences, and tetracycline inactivates the transactivator and inhibits transcription. This system has been used in plants (Weinmann et al. (1994) Plant J 5:559-569), rat hearts (Fishman et al. (1994) J Clin Invest 93:1864-1868) and mice (Furth et al. (1994) PNAS 91:9302-9306). However, there were indications that the chimeric tTA fusion protein was toxic to cells at levels required for efficient gene regulation (Bohl et al. (1996) Nat Med 3:299-305).

Useful tet operator containing promoters further include those known in the art (see, e.g., Matzke et al. (2003) Plant Mol Biol Rep 21:9-19; Padidam (2003) Curr Op Plant Biol 6:169-177; Gatz & Quail (1988) PNAS 85:1394-1397; Ulmasov et al. (1997) Plant Mol Biol 35:417-424; Weinmann et al. (1994) Plant J 5:559-569). One or more tet operator sequences can be added to a promoter in order to produce a tetracycline inducible promoter. In some examples up to 7 tet operators have been introduced upstream of a minimal promoter sequence and a TetR::VP16 activation domain fusion applied in trans activates expression only in the absence of inducer (Weinmann et al. (1994) Plant J 5:559-569; Love et al. (2000) Plant J 21:579-588). A widely tested tetracycline regulated expression system for plants using the CaMV 35S promoter was developed (Gatz et al. (1992) Plant J 2:397-404) having three tet operators introduced near the TATA box (3XOpT 35S). The 3XOpT 35S promoter generally functioned in tobacco and potato, however toxicity and poor plant phenotype in tomato and Arabidopsis (Gatz (1997) Ann Rev Plant Physiol Plant Mol Biol 48:89-108; Corlett et al. (1996) Plant Cell Environ 19:447-454) were also reported. Another factor is that the tetracycline-related chemistry is rapidly degraded in the light, which tends to confine its use to testing in laboratory conditions.

One characteristic of a chemically regulated gene switch is its sensitivity to cognate ligand. One way to potentially improve ligand response when using a negatively controlled system as described here is to auto-regulate expression of the repressor. It had been mathematically predicted that negative auto-regulation would not only dampen fluctuations in gene expression but also enhance signal response time in regulatory circuits involving repressor molecules (Savageau (1974) Nature 252:542-549). This principle was demonstrated in E. coli using synthetic gene circuitry (Rosenfeld et al. (2002) J Mol Biol 323:785-793). Most recently Nevozhay et al. extended this finding to a lower eukaryote (yeast) by comparing synthetic gene networks built with and without an auto-regulated tetracycline repressor and fluorescent protein reporter system (Nevozhay (2009) Proc Natl Acad Sci USA 106:5123-5128).

The modular architecture of repressor proteins and the commonality of helix-turn-helix DNA binding domains allows for the creation of SuR polypeptides having altered DNA binding specificity. For example, the DNA binding specificity can be altered by fusing a SuR ligand binding domain to an alternate DNA binding domain. For example, the DNA binding domain from TetR class D can be fused to a SuR ligand binding domain to create SuR polypeptides that specifically bind to polynucleotides comprising a class D tetracycline operator. In some examples a DNA binding domain variant or derivative can be used. For example, a DNA binding domain from a TetR variant that specifically recognizes a tetO-4C operator or a tetO-6C operator could be used (Helbl & Hillen (1998) J Mol Biol 276:313-318; Helbl et al. (1998) J Mol Biol 276:319-324. The four helix bundle formed by helices α8 and α10 in both subunits can be substituted to ensure dimerization specificity when targeting two different operator specific repressor variants in the same cell to prevent heterodimerization (e.g., Rossi et al. (1998) Nat Genet 20:389-393; Berens & Hillen (2003) Eur J Biochem 270:3109-3121). In another example, the DNA binding domain from LexA repressor was fused to GAL4 wherein this hybrid protein recognized LexA operators in both E. coli and yeast (Brent & Ptashne (1985) Cell 43:729-736). In another example, all of the presumptive DNA binding or DNA-recognition R-groups of the 434 repressor were replaced by the corresponding positions of the P22 repressor. Operator binding specificity of the hybrid repressor 434R[α3(P22R)] was tested both in vivo and in vitro and each test showed that this targeted modification of 434 shifted the DNA binding specificity from 434 operator to P22 operator (Wharton & Ptashne (1985) Nature 316:601-605). This work was further extended by creating a heterodimer of wild type 434R and 434R[α3(P22R)] which then specifically recognized a chimeric P22/434 operator sequence (Hollis et al. (1988) PNAS 85:5834-5838). In another example, the N-terminal half of the AraC protein was fused to the LexA repressor DNA binding domain. The resulting AraC:LexA chimera dimerized, bound LexA operator, and repressed expression of a LexA operator:β-galactosidase fusion gene in an arabinose-responsive manner (Bustos & Schleif (1993) PNAS 90:5638-5642).

For convenience and high throughput it will often be desirable to screen/select for desired modified nucleic acids in a microorganism, such as in a bacteria such as E. coli, or unicellular eukaryote such as yeast including S. cerevisiae, S. pombe, P. pastoris or protists such as Chlamydomonas, or in model cell systems such as SF9, Hela, CHO, BMS, BY2, or other cell culture systems. In some instances, screening in plant cells or plants may be desirable, including plant cell or explant culture systems or model plant systems such as Arabidopsis, or tobacco. In some examples throughput is increased by screening pools of host cells expressing different modified nucleic acids, either alone or as part of a gene fusion construct. Any pools showing significant activity can be deconvoluted to identify single clones expressing the desirable activity.

Recombinant constructs comprising one or more of nucleic acid sequences such as a gene switch, a plant promoter comprising at least one tet operator, a polynucleotide encoding a SuR polypeptide, and/or a polynucleotide of interest are provided. The constructs comprise a vector, such as, a plasmid, a cosmid, a phage, a virus, a bacterial artificial chromosome (BAC), a yeast artificial chromosome (YAC), or the like, into which a polynucleotide has been inserted. In some examples, the construct further comprises regulatory sequences, including, for example, a promoter, operably linked to the sequence. Suitable vectors are well known and include chromosomal, non-chromosomal and synthetic DNA sequences, such as derivatives of SV40; bacterial plasmids; replicons; phage DNA; baculovirus; yeast plasmids; vectors derived from combinations of plasmids and phage DNA, viral DNA such as vaccinia, adenovirus, fowl pox virus, pseudorabies, adenovirus, adeno-associated viruses, retroviruses, geminiviruses, TMV, PVX, other plant viruses, Ti plasmids, Ri plasmids and many others.

The vectors may optionally contain one or more selectable marker genes to provide a phenotypic trait for selection of transformed host cells. Usually, the selectable marker gene will encode antibiotic or herbicide resistance. Suitable genes include those coding for resistance to the antibiotic spectinomycin or streptomycin (e.g., the aadA gene), the streptomycin phosphotransferase (SPT) gene for streptomycin resistance, the neomycin phosphotransferase (NPTII or NPTIII) gene kanamycin or geneticin resistance, the hygromycin phosphotransferase (HPT) gene for hygromycin resistance. Additional selectable marker genes include dihydrofolate reductase or neomycin resistance for eukaryotic cell culture and tetracycline or ampicillin resistance. Genes coding for resistance to herbicides include those which act to inhibit the action of glutamine synthase, such as phosphinothricin or basta (e.g., the bar gene), EPSPS, GOX, or GAT which provide resistance to glyphosate, mutant ALS (acetolactate synthase) which provides resistance to sulfonylurea type herbicides or any other known genes.

In bacterial systems a number of expression vectors are available. Such vectors include, but are not limited to, multifunctional E. coli cloning and expression vectors such as BLUESCRIPT (Stratagene); pIN vectors (Van Heeke & Schuster (1989) J Biol Chem 264:5503-5509); pET vectors (Novagen, Madison Wis.) and the like. Similarly, in S. cerevisiae a number of vectors containing constitutive or inducible promoters such as alpha factor, alcohol oxidase and PGH may be used for production of polypeptides. For reviews, see, Ausubel & Grant et al. (1987) Meth Enzymol 153:516-544. A variety of expression systems can be used in mammalian host cells, including viral-based systems, such as adenovirus and rous sarcoma virus (RSV) systems. Any number of commercially or publicly available expression systems or derivatives thereof can be used.

In plant cells expression can be driven from an expression cassette integrated into a plant chromosome, or an organelle, or cytoplasmically from an episomal or viral nucleic acid. Numerous plant derived regulatory sequences have been described, including sequences which direct expression in a tissue specific manner, e.g., TobRB7, patatin B33, GRP gene promoters, the rbcS-3A promoter and the like. Alternatively, high level expression can be achieved by transiently expressing exogenous sequences of a plant viral vector, e.g., TMV, BMV, geminiviruses including WDV and the like.

Typical vectors useful for expression of nucleic acids in higher plants are known including vectors derived from the tumor-inducing (Ti) plasmid of Agrobacterium tumefaciens described by Rogers et al. (1987) Meth Enzymol 153:253-277. Exemplary A. tumefaciens vectors include plasmids pKYLX6 and pKYLX7 of Schardl et al. (1987) Gene 61:1-11, and Berger et al. (1989) PNAS 86:8402-8406 and plasmid pB101.2 (e.g., available from Clontech Laboratories, Palo Alto, Calif.). A variety of known plant viruses can be employed as vectors including cauliflower mosaic virus (CaMV), geminiviruses, brome mosaic virus and tobacco mosaic virus.

The gene switch, a promoter, a TetOp repressible plant promoter, a recombinase, and/or the SuR may be used to control expression of a polynucleotide of interest. The polynucleotide of interest may be any sequence of interest, including but not limited to transcription regulatory elements, translation regulatory elements, centromere elements, telomere elements, sequences encoding a polypeptide, encoding an mRNA, encoding a tRNA, encoding an rRNA, encoding a sequence that directs gene silencing, a ribozyme, a fusion protein, a replicating vector, a screenable marker, and the like. Expression of the polynucleotide of interest may be used to induce expression of an encoding RNA and/or polypeptide, or conversely to suppress expression of an encoded RNA, RNA target sequence, and/or polypeptide. In specific examples the polynucleotide of interest comprises a sequence that directs gene silencing, including but not limited to encoding an RNAi precursor, encoding an active RNAi agent, a miRNA, an antisense polynucleotide, a ribozyme, or any other silencing molecule and combinations thereof. In specific examples, the polynucleotide sequence may comprise a polynucleotide encoding a plant hormone, plant defense protein, a nutrient transport protein, a biotic association protein, a desirable input trait, a desirable output trait, a stress resistance gene, a herbicide resistance gene, a disease/pathogen resistance gene, a male sterility, a developmental gene, a regulatory gene, a DNA repair gene, a transcriptional regulatory gene, a biosynthetic polypeptide, or any other polynucleotide and/or polypeptide of interest and combinations thereof. A biosynthetic polypeptide is any polypeptide involved in any biological, cellular, metabolic, synthetic, and/or catabolic pathway in a cell. In some examples, the polynucleotide of interest encodes a polypeptide that specifically binds to a target nucleic acid sequence, examples of which include but are not limited to polynucleotides encoding recombinases, integrases, excisionases, transposases, repressors, reverse repressors, activators, nucleases, endonucleases, exonucleases, homing endonucleases, zinc-finger proteins, zinc-finger nucleases, transcription factors, polymerases, ligases, and the like. In some examples, a polypeptide that specifically binds to a target nucleic acid sequence cuts at least one strand of the target nucleic acid at or near a specific sequence defined by sequence composition and/or proximity to a specific sequence composition (e.g., a type IIS restriction nuclease, such as Fokl). For example, a polynucleotide of interest can encode a polypeptide that cuts a DNA nucleic acid molecule at a specific sequence. In some examples, the polynucleotide of interest encodes a polynucleotide that specifically binds to a target nucleic acid sequence, examples of which include but are not limited to an antisense polynucleotide, a miRNA precursor, a miRNA, and the like.

Specific binding refers to binding, duplexing, or hybridization of a molecule to a specific target molecule at a level that is significantly higher than binding to a non-target molecule. Generally, specific binding is a level at least 2-fold higher than non-specific background binding. Specific binding includes binding of a chemical molecule, polypeptide, or polynucleotide to any of a target chemical, target polypeptide, or target polynucleotide.

Any promoter(s) can be used in the compositions and methods. For example, a polynucleotide encoding a SuR polypeptide, a recombinase, a polynucleotide of interest, or any other sequence can be operably linked to a constitutive, a tissue-preferred, an inducible, a developmentally, a temporally and/or a spatially regulated or other promoters including those from plant viruses or other pathogens which function in a plant cell. A variety of promoters useful in plants is reviewed in Potenza et al. (2004) In Vitro Cell Dev Biol Plant 40:1-22. Exemplary promoters include but are not limited to a 35S CaMV promoter (Odell et al. (1995) Nature 313:810-812), a S-adenosylmethionine synthase promoter (SAMS) (e.g., those disclosed in U.S. Pat. No. 7,217,858 and US2008/0026466), a Mirabilis mosaic virus promoter (e.g., Dey & Maiti (1999) Plant Mol Biol 40:771-782; Dey & Maiti (1999) Transgenics 3:61-70), an elongation factor promoter (e.g., US2008/0313776 and US2009/0133159), a banana streak virus promoter, an actin promoter (e.g., McElroy et al. (1990) Plant Cell 2:163-171), a TobRB7 promoter (e.g., Yamamoto et al. (1991) Plant Cell 3:371), a patatin promoter (e.g., patatin B33, Martin et al. (1997) Plant J 11:53-62), a ribulose 1,5-bisphosphate carboxylase promoter (e.g., rbcS-3A, see, for example Fluhr et al. (1986) Science 232:1106-1112, and Pellingrinischi et al. (1995) Biochem Soc Trans 23:247-250), an ubiquitin promoter (e.g., Christensen et al. (1992) Plant Mol Biol 18:675-689, and Christensen & Quail (1996) Transgen Res 5:213-218), a metallothionin promoter (e.g., US2010/0064390), a Rab17 promoter (e.g., Vilardell et al. (1994) Plant Mol Biol 24:561-569), a conglycinin promoter (e.g., Chamberland et al. (1992) Plant Mol Biol 19:937-949), a plasma membrane intrinsic (PIP) promoter (e.g., Alexandersson et al. (2009) Plant J 61:650-660), a lipid transfer protein (LTP) promoter (e.g., US2009/0158464, US2009/0070893, and US2008/0295201), a gamma zein promoter (e.g., Uead et al. (1994) Mol Cell Biol 14:4350-4359), a gamma kafarin promoter (e.g., Mishra et al. (2008) Mol Biol Rep 35:81-88), a globulin promoter (e.g., Liu et al. (1998) Plant Cell Rep 17:650-655), a legumin promoter (e.g., U.S. Pat. No. 7,211,712), an early endosperm promoter (EEP) (e.g., US2007/0169226 and US2009/0227013), a B22E promoter (e.g., Klemsdal et al. (1991) Mol Gen Genet 228:9-16), an oleosin promoter (e.g., Plant et al. (1994) Plant Mol Biol 25:193-205), an early abundant protein (EAP) promoter (e.g., U.S. Pat. No. 7,321,031), a late embryogenesis abundant (LEA) protein (e.g., Hva1, Straub et al. (1994) Plant Mol Biol 26:617-630; Dhn and WSI18, Xiao & Xue (2001) Plant Cell Rep 20:667-673), In2-2 promoter (De Veylder et al. (1997) Plant Cell Physiol 38:568-577), a glutathione S-transferase (GST) promoter (e.g., WO93/01294), a PR promoter (e.g., Cao et al. (2006) Plant Cell Rep 6:554-560, and Ono et al. (2004) Biosci Biotech Biochem 68:803-807), an ACE1 promoter (e.g., Mett et al. (1993) Proc Natl Acad Sci USA 90:4567-4571), a steroid responsive promoter (e.g., Schena et al. (1991) Proc Natl Acad Sci USA 88:10421-10425, and McNellis et al. (1998) Plant J 14:247-257), an ethanol-inducible promoter (e.g., AlcA, Caddick et al. (1988) Nat Biotechnol 16:177-180), an estradiol-inducible promoter (e.g., Bruce et al. (2000) Plant Cell 12:65-79), an XVE estradiol-inducible promoter (e.g., Zao et al. (2000) Plant J 24: 265-273), a VGE methoxyfenozide-inducible promoter (e.g., Padidam et al. (2003) Transgen Res 12:101-109), or a TGV dexamethasone-inducible promoter (e.g., Bohner et al. (1999) Plant J 19:87-95).

Any polynucleotide, including isolated or recombinant polynucleotides of interest, polynucleotides encoding SuRs, recombinase sites, polynucleotides encoding a recombinase, regulatory regions, introns, promoters, and promoters comprising TetOp sequences may be obtained and their nucleotide sequence determined, by any standard method. The polynucleotides may be chemically synthesized in their full-length or assembled from chemically synthesized oligonucleotides (Kutmeier et al. (1994) BioTechniques 17:242). Assembly from oligonucleotides typically involves synthesis of overlapping oligonucleotides, annealing and ligating of those oligonucleotides and PCR amplification of the ligated product. Alternatively, a polynucleotide may be isolated or generated from a suitable source including suitable source a cDNA library generated from tissue or cells, a genomic library, or directly isolated from a host by PCR amplification using specific primers to the 3′ and 5′ ends of the sequence or by cloning using an nucleotide probe specific for the polynucleotide of interest. Amplified nucleic acid molecules generated by PCR may then be cloned into replicable cloning vectors using standard methods. The polynucleotide may be further manipulated using any standard methods including recombinant DNA techniques, vector construction, mutagenesis and PCR (see, e.g., Sambrook et al. (1990) Molecular Cloning, A Laboratory Manual, 2d Ed., Cold Spring Harbor Laboratory, Cold Spring Harbor, N.Y.; Ausubel et al., Eds. (1998) Current Protocols in Molecular Biology, John Wiley and Sons, NY).

A polynucleotide, polypeptide or other component is “isolated” when it is partially or completely separated from components with which it is normally associated (other proteins, nucleic acids, cells, synthetic reagents, etc.). A nucleic acid or polypeptide is “recombinant” when it is artificial or engineered, or derived from an artificial or engineered protein or nucleic acid. For example, a polynucleotide that is inserted into a vector or any other heterologous location, e.g, in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant. For example, the present invention encompasses recombinant polynucleotides comprising repressible promoters comprising at least one operator sequence or repressible promoters operably linked to a polynucleotide encoding a sulfonylurea-responsive repressor.

In some examples, a recombinant polynucleotide may comprise a repressible promoter operably linked to a polynucleotide encoding a sulfonylurea-responsive repressor, where the repressible promoter comprises a tet operator. In some examples. the encoded sulfonylurea-responsive repressor comprises an amino acid sequence of any one of SEQ ID NO:3-419, or an amino acid sequence having at least 85% (e.g., at least 85%, 90%, 95%, 97%, 99%, 100%) sequence identity to any one of SEQ ID NO:3-419. The repressible promoter can comprise an actin promoter, an MMV promoter, a dMMV promoter, an MP1 promoter, or a BSV promoter operably linked to at least one operator sequence. In some examples, the repressible promoter comprises a polynucleotide sequence as set forth in SEQ ID NO:855, 856, 857, 858, 859, 860 or 862 or, as described herein, a polynucleotide sequence having at least 95% sequence identity to SEQ ID NO:855, 856, 857, 858, 859, 860 or 862.

Any method for introducing a sequence into a cell or organism can be used, as long as the polynucleotide or polypeptide gains access to the interior of at least one cell. Methods for introducing sequences into plants are known and include, but are not limited to, stable transformation, transient transformation, virus-mediated methods, and sexual breeding. Stably incorporated indicates that the introduced polynucleotide is integrated into a genome and is capable of being inherited by progeny. Transient transformation indicates that an introduced sequence does not integrate into a genome such that it is heritable by progeny from the host. Any means can be used to bring together any gene switch element or combinations thereof, for example a SuR and a polynucleotide of interest operably linked to a promoter comprising a TetOp, including, e.g., stable transformation, transient delivery, cell fusion, sexual crossing or any combination thereof.

Transformation protocols as well as protocols for introducing polypeptides or polynucleotide sequences into plants may vary depending on the type of plant or plant cell targeted for transformation. Suitable methods of introducing polypeptides and polynucleotides into plant cells include microinjection (Crossway et al. (1986) Biotechniques 4:320-334 and U.S. Pat. No. 6,300,543), electroporation (Riggs et al. (1986) PNAS 83:5602-5606, Agrobacterium-mediated transformation (U.S. Pat. Nos. 5,563,055 & 5,981,840), direct gene transfer (Paszkowski et al. (1984) EMBO J 3:2717-2722), ballistic particle acceleration (U.S. Pat. Nos. 4,945,050, 5,879,918, 5,886,244 & 5,932,782; Tomes et al. (1995) in Plant Cell, Tissue and Organ Culture: Fundamental Methods, ed. Gamborg and Phillips (Springer-Verlag, Berlin); McCabe et al. (1988) Biotechnology 6:923-926). Also see, Weissinger et al. (1988) Ann Rev Genet 22:421-477; Sanford et al. (1987) Particulate Science and Technology 5:27-37; Christou et al. (1988) Plant Physiol 87:671-674; Finer & McMullen (1991) In Vitro Cell Dev Biol 27P:175-182 (soybean); Singh et al. (1998) Theor Appl Genet 96:319-324; Datta et al. (1990) Biotechnology 8:736-740; Klein et al. (1988) PNAS 85:4305-4309; Klein et al. (1988) Biotechnology 6:559-563; U.S. Pat. Nos. 5,240,855, 5,322,783 & 5,324,646; Klein et al. (1988) Plant Physiol 91:440-444; Fromm et al. (1990) Biotechnology 8:833-839; Hooykaas-Van Slogteren et al. (1984) Nature 311:763-764; U.S. Pat. No. 5,736,369; Bytebier et al. (1987) PNAS 84:5345-5349; De Wet et al. (1985) in The Experimental Manipulation of Ovule Tissues, ed. Chapman et al. (Longman, New York), pp. 197-209; Kaeppler et al. (1990) Plant Cell Rep 9:415-418; Kaeppler et al. (1992) Theor Appl Genet 84:560-566; D'Halluin et al. (1992) Plant Cell 4:1495-1505; Li et al. (1993) Plant Cell Rep 12:250-255; Christou & Ford (1995) Ann Bot 75:407-413 and Osjoda et al. (1996) Nat Biotechnol 14:745-750. Alternatively, polynucleotides may be introduced into plants by contacting plants with a virus, or viral nucleic acids. Methods for introducing polynucleotides into plants via viral DNA or RNA molecules are known, see, e.g., U.S. Pat. Nos. 5,889,191, 5,889,190, 5,866,785, 5,589,367, 5,316,931; and Porta et al. (1996) Mol Biotech 5:209-221.

The term plant includes plant cells, plant protoplasts, plant cell tissue cultures, plant cells or plant tissue cultures from which a plant can be regenerated, plant calli, plant clumps and plant cells that are intact in plants or parts of plants such as embryos, pollen, ovules, seeds, endosperm, meristem, leaves, flowers, branches, fruit, kernels, ears, cobs, husks, stalks, roots, root tips, anthers and the like. Progeny, variants and mutants of the regenerated plants are also included.

In some examples, a SuR may be introduced into a plastid, either by transformation of the plastid or by directing a SuR transcript or polypeptide into the plastid. Any method of transformation, nuclear or plastid, can be used, depending on the desired product and/or use. Plastid transformation provides advantages including high transgene expression, control of transgene expression, ability to express polycistronic messages, site-specific integration via homologous recombination, absence of transgene silencing and position effects, control of transgene transmission via uniparental plastid gene inheritance and sequestration of expressed polypeptides in the organelle which can obviate possible adverse impacts on cytoplasmic components (e.g., see, reviews including Heifetz (2000) Biochimie 82:655-666; Daniell et al. (2002) Trends Plant Sci 7:84-91; Maliga (2002) Curr Op Plant Biol 5:164-172; Maliga (2004) Ann Rev Plant Biol 55-289-313; Daniell et al. (2005) Trends Biotechnol 23:238-245; and, Verma & Daniell (2007) Plant Physiol 145:1129-1143).

Methods and compositions of plastid transformation are well known, for example, transformation methods include (Boynton et al. (1988) Science 240:1534-1538; Svab et al. (1990) PNAS 87:8526-8530; Svab et al. (1990) Plant Mol Biol 14:197-205; Svab et al. (1993) PNAS 90:913-917; Golds et al. (1993) Bio/Technology 11:95-97; O'Neill et al. (1993) Plant J 3:729-738; Koop et al. (1996) Planta 199:193-201; Kofer et al. (1998) In Vitro Plant 34:303-309; Knoblauch et al. (1999) Nat Biotechnol 17:906-909); as well as plastid transformation vectors, elements, and selection (Newman et al. (1990) Genetics 126:875-888; Goldschmidt-Clermont, (1991) Nucl Acids Res 19:4083-4089; Carrer et al. (1993) Mol Gen Genet 241:49-56; Svab et al. (1993) PNAS 90:913-917; Verma & Daniell (2007) Plant Physiol 145:1129-1143).

Methods and compositions for controlling gene expression in plastids are well known including (McBride et al. (1994) PNAS 91:7301-7305; Lössl et al. (2005) Plant Cell Physiol 46:1462-1471; Heifetz (2000) Biochemie 82:655-666; Surzycki et al. (2007) PNAS 104:17548-17553; U.S. Pat. Nos. 5,576,198 and 5,925,806; WO 2005/0544478), as well as methods and compositions to import polynucleotides and/or polypeptides into a plastid, including translational fusion to a transit peptide (e.g., Comai et al. (1988) J Biol Chem 263:15104-15109).

The SuR polynucleotides and polypeptides provide a means for regulating plastid gene expression via a chemical inducer that readily enters the cell. For example, using the T7 expression system for chloroplasts (McBride et al. (1994) PNAS 91:7301-7305) the SuR could be used to control nuclear T7 polymerase expression. Alternatively, a SuR-regulated promoter could be integrated into the plastid genome and operably linked to the polynucleotide(s) of interest and the SuR expressed and imported from the nuclear genome, or integrated into the plastid. In all cases, application of a sulfonylurea compound is used to efficiently regulate the polynucleotide(s) of interest. A sulfonylurea compound can be applied according to any appropriate method known in the art. For example, a sulfonylurea compound can be applied by foliar application, root drench application, pre-emergence application, post-emergence application, or seed treatment application.

The repressible promoters provide a means for regulating plastid gene expression via a chemical inducer that readily enters the cell. A TetR or SuR-regulated promoter, including but not limited to SEQ ID NO:855-860 or, as described herein, a promoter having at least 95% sequence identity to SEQ ID NO:855-860, could be integrated into the plastid genome and operably linked to the polynucleotide(s) of interest and the repressor expressed and imported from the nuclear genome, or integrated into the plastid. In all cases, application of a tetracycline compound or a sulfonylurea compound is used to efficiently regulate the polynucleotide(s) of interest.

Any type of cell and/or organism, prokaryotic or eukaryotic, can be used with the gene switch components, gene switch compositions and/or the methods. For example, any bacterial cell system can be transformed with the compositions. For example, methods of E. coli, Agrobacterium and other bacterial cell transformation, plasmid preparation and the use of phages are detailed, for example, in Current Protocols in Molecular Biology (Ausubel, et al., (eds.) (1994) a joint venture between Greene Publishing Associates, Inc. and John Wiley & Sons, Inc.).

The gene switch components, gene switch compositions and/or systems can be used with any eukaryotic cell line, including yeasts, protists, algae, insect cells, avian cells or mammalian cells. For example, many commercially and/or publicly available strains of S. cerevisiae are available, as are the plasmids used to transform these cells. For example, strains are available from the American Type Culture Collection (ATCC, Manassas, Va.) and include the Yeast Genetic Stock Center inventory, which moved to the ATCC in 1998. Other yeast lines, such as S. pombe and P. pastoris, and the like are also available. For example, methods of yeast transformation, plasmid preparation, and the like are detailed, for example, in Current Protocols in Molecular Biology (Ausubel et al. (eds.) (1994) a joint venture between Greene Publishing Associates, Inc. and John Wiley & Sons, Inc., see Unit 13 in particular). Transformation methods for yeast include spheroplast transformation, electroporation, and lithium acetate methods. A versatile, high efficiency transformation method for yeast is described by Gietz & Woods ((2002) Methods Enzymol 350:87-96) using lithium acetate, PEG 3500 and carrier DNA.

The gene switch components, and/or gene switch compositions can be used in mammalian cells, such as CHO, HeLa, BALB/c, fibroblasts, mouse embryonic stem cells and the like. Many commercially available competent cell lines and plasmids are well known and readily available, for example from the ATCC (Manassas, Va.). Isolated polynucleotides for transformation and transformation of mammalian cells can be done by any method known in the art. For example, methods of mammalian and other eukaryotic cell transformation, plasmid preparation, and the use of viruses are detailed, for example, in Current Protocols in Molecular Biology (Ausubel et al. (eds.) (1994) a joint venture between Greene Publishing Associates, Inc. and John Wiley & Sons, Inc., see, Unit 9 in particular). For example, many methods are available, such as calcium phosphate transfection, electroporation, DEAE-dextran transfection, liposome-mediated transfection, microinjection, as well as viral techniques.

Any plant species can be used with the gene switch components, gene switch compositions, and/or methods, including, but not limited to, monocots and dicots. Examples of plants include, but are not limited to, corn (Zea mays), Brassica spp. (e.g., B. napus, B. rapa, B. juncea), castor, palm, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), Arabidopsis thaliana, oats (Avena spp.), barley (Hordeum spp.), leguminous plants such as guar beans, locust bean, fenugreek, garden beans, cowpea, mungbean, fava bean, lentils, and chickpea, vegetables, ornamentals, grasses and conifers. Vegetables include tomatoes (Lycopersicon esculentum), lettuce (e.g., Lactuca sativa), green beans (Phaseolus vulgaris), lima beans (Phaseolus limensis), peas (Pisium spp., Lathyrus spp.), and Cucumis species such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers include pines, for example, loblolly pine (Pinus taeda), slash pine (Pinus elliotii), ponderosa pine (Pinus ponderosa), lodgepole pine (Pinus contorta), and Monterey pine (Pinus radiata), Douglas fir (Pseudotsuga menziesii); Western hemlock (Tsuga canadensis), Sitka spruce (Picea glauca), redwood (Sequoia sempervirens), true firs such as silver fir (Abies amabilis) and balsam fir (Abies balsamea) and cedars such as Western red cedar (Thuja plicata) and Alaska yellow cedar (Chamaecyparis nootkatensis).

The plant cells and/or tissue that have been transformed may be grown into plants using conventional methods (see, e.g., McCormick et al. (1986) Plant Cell Rep 5:81-84). These plants may then be grown and self-pollinated, backcrossed, and/or outcrossed, and the resulting progeny having the desired characteristic identified. Two or more generations may be grown to ensure that the characteristic is stably maintained and inherited and then seeds harvested. In this manner transformed seed having a gene switch component, a repressor, a repressible promoter, a gene switch system, a polynucleotide of interest, a recombinase, a recombination event end-product, and/or a polynucleotide encoding a SuR stably incorporated into their genome are provided. A plant and/or a seed having stably incorporated the DNA construct can be further characterized for expression, agronomics and copy number.

Sequence identity may be used to compare the primary structure of two polynucleotides or polypeptide sequences, describe the primary structure of a first sequence relative to a second sequence, and/or describe sequence relationships such as variants and homologues. Sequence identity measures the residues in the two sequences that are the same when aligned for maximum correspondence. Sequence relationships can be analyzed using computer-implemented algorithms. The sequence relationship between two or more polynucleotides or two or more polypeptides can be determined by computing the best alignment of the sequences and scoring the matches and the gaps in the alignment, which yields the percent sequence identity and the percent sequence similarity. Polynucleotide relationships can also be described based on a comparison of the polypeptides each encodes. Many programs and algorithms for comparison and analysis of sequences are known. Unless otherwise stated, sequence identity/similarity values provided herein refer to the value obtained using GAP Version 10 (GCG, Accelrys, San Diego, Calif.) using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix (Henikoff & Henikoff (1992) PNAS 89:10915-10919). GAP uses the algorithm of Needleman & Wunsch (1970) J Mol Biol 48:443-453, to find the alignment of two complete sequences that maximizes the number of matches and minimizes the number of gaps.

Alternatively, polynucleotides and/or polypeptides can be evaluated using other sequence tools. For example, polynucleotides and/or polypeptides can be evaluated using a BLAST alignment tool. A local alignment gaps consists simply of a pair of sequence segments, one from each of the sequences being compared. A modification of Smith-Waterman or Sellers algorithms will find all segment pairs whose scores cannot be improved by extension or trimming, called high-scoring segment pairs (HSPs). The results of the BLAST alignments include statistical measures to indicate the likelihood that the BLAST score can be expected from chance alone. The raw score, S, is calculated from the number of gaps and substitutions associated with each aligned sequence wherein higher similarity scores indicate a more significant alignment. Substitution scores are given by a look-up table (see PAM, BLOSUM). Gap scores are typically calculated as the sum of G, the gap opening penalty and L, the gap extension penalty. For a gap of length n, the gap cost would be G+Ln. The choice of gap costs, G and L is empirical, but it is customary to choose a high value for G (10-15) and a low value for L (1-2). The bit score, S′, is derived from the raw alignment score S in which the statistical properties of the scoring system used have been taken into account. Bit scores are normalized with respect to the scoring system, therefore they can be used to compare alignment scores from different searches. The E-Value, or expected value, describes the likelihood that a sequence with a similar score will occur in the database by chance. It is a prediction of the number of different alignments with scores equivalent to or better than S that are expected to occur in a database search by chance. The smaller the E-Value, the more significant the alignment. For example, an alignment having an E value of e⁻¹¹⁷ means that a sequence with a similar score is very unlikely to occur simply by chance. Additionally, the expected score for aligning a random pair of amino acid is required to be negative, otherwise long alignments would tend to have high score independently of whether the segments aligned were related. Additionally, the BLAST algorithm uses an appropriate substitution matrix, nucleotide or amino acid and for gapped alignments uses gap creation and extension penalties. For example, BLAST alignment and comparison of polypeptide sequences are typically done using the BLOSUM62 matrix, a gap existence penalty of 11 and a gap extension penalty of 1. Unless otherwise stated, scores reported from BLAST analyses were done using the BLOSUM62 matrix, a gap existence penalty of 11 and a gap extension penalty of 1.

UniProt protein sequence database is a repository for functional and structural protein data and provides a stable, comprehensive, fully classified, richly and accurately annotated protein sequence knowledgebase, with extensive cross-references and querying interfaces freely accessible to the scientific community. The UniProt site has a tool, UniRef, which provides a cluster of proteins have 50%, 90% or 100% sequence identity to a protein sequence of interest from the database. For example, using TetR(B) (UniProt reference P04483) gives a cluster of 18 proteins having 90% sequence identity to P04483.

The properties, domains, motifs and function of tetracycline repressors are well known, as are standard techniques and assays to evaluate any derived repressor comprising one or more amino acid substitutions. The structure of the class D TetR protein comprises 10 alpha helices with connecting loops and turns. The 3 N-terminal helices form the DNA-binding HTH domain, which has an inverse orientation as compared to HTH motifs in other DNA-binding proteins. The core of the protein, formed by helices 5-10, comprises the dimerization interface domain, and for each monomer comprises the binding pocket for ligand/effector and divalent cation cofactor (Kisker et al. (1995) J Mol Biol 247:260-180; Orth et al. (2000) Nat Struct Biol 7:215-219). Any amino acid change may comprise a non-conservative or conservative amino acid substitution. Conservative substitutions generally refer to exchanging one amino acid with another having similar chemical and/or structural properties (see, e.g., Dayhoff et al. (1978) Atlas of Protein Sequence and Structure, Natl Biomed Res Found, Washington, D.C.). Different clustering of amino acids by similarity have been developed depending on the property evaluated, such as acidic vs. basic, polar vs. non-polar, amphipathic and the like and be used when evaluating the possible effect of any substitution or combination of substitutions.

Numerous variants of TetR have been identified and/or derived and extensively studied. In the context of the tetracycline repressor system, the effects of various mutations, modifications and/or combinations thereof have been used to extensively characterize and/or modify the properties of tetracycline repressors, such as cofactor binding, ligand binding constants, kinetics and dissociation constants, operator binding sequence constraints, cooperativity, binding constants, kinetics and dissociation constants and fusion protein activities and properties. Variants include TetR variants with a reverse phenotype of binding the operator sequence in the presence of tetracycline or an analog thereof, variants having altered operator binding properties, variants having altered operator sequence specificity and variants having altered ligand specificity and fusion proteins. See, for example, Isackson & Bertrand (1985) PNAS 82:6226-6230; Smith & Bertrand (1988) J Mol Biol 203:949-959; Altschmied et al. (1988) EMBO J 7:4011-4017; Wissmann et al. (1991) EMBO J 10:4145-4152; Baumeister et al. (1992) J Mol Biol 226:1257-1270; Baumeister et al. (1992) Proteins 14:168-177; Gossen & Bujard (1992) PNAS 89:5547-5551; Wasylewski et al. (1996) J Protein Chem 15:45-58; Berens et al. (1997) J Biol Chem 272:6936-6942; Baron et al. (1997) Nucl Acids Res 25:2723-2729; Helbl & Hillen (1998) J Mol Biol 276:313-318; Urlinger et al. (2000) PNAS 97:7963-7968; Kamionka et al. (2004) Nucl Acids Res 32:842-847; Bertram et al. (2004) J Mol Microbiol Biotechnol 8:104-110; Scholz et al. (2003) J Mol Biol 329: 217-227; and US2003/0186281.

### EXAMPLES

The following examples are provided to illustrate some embodiments of the invention, but should not be construed as defining or otherwise limiting any aspect, embodiment, element or any combinations thereof. Modifications of any aspect, embodiment, element or any combinations thereof are apparent to a person of skill in the art.

### Example 1

### Sulfonylurea-Responsive Repressors (SuRs)

### A. Computational Modeling

The 3-D crystal structures of the class D tetracycline repressor (isolated from E. coli; TET-bound dimer, 1DU7 (Orth et al. (2000) Nat Struct Biol 7:215-219); and DNA-bound dimer, 1QPI (Orth et al. (2000) Nat Struct Biol 7:215-219), were used as the design scaffold for computational replacement of the tetracycline (TET) molecule by the thifensulfuron-methyl (Ts, Harmony®) molecule in the ligand binding pocket. TET and sulfonylureas (SUs) are generally similar in size and have aromatic ring-based structures with hydrogen bond donors and acceptors. However, there are notable differences between the tetracycline family and SU family of molecules. TET is internally rigid and fairly flat, with one highly-hydrogen-bonding face with hydroxyls and ketones, log P ˜−0.3. Sulfonylureas (SUs) are more highly flexible and aromatic, with a core sulfonyl-urea moiety typically connecting a substituted benzene, pyridine, or thiophene (as in the case of Harmony®) on one side with a substituted pyrimidine or 1,3,5-triazine on the other side. Although having different functional groups, the logP of Harmony® is similar (˜0.02 at pH 7) to that of TET. A best-posed Harmony® molecule was positioned by molecular modeling in the TetR binding pocket in silico. Based on this model, seventeen amino acid residue positions (60, 64, 82, 86, 100, 104, 105, 113, 116, 134, 135, 138 and 139 from monomer A and positions 147, 151, 174 and 177 from monomer B, using TetR(B) numbering) were determined to be in sufficiently close proximity to a docked Harmony® as to be recruited into a binding surface. Computational side-chain optimization was employed to design sets of amino acids at each of the 17 positions deemed to be most compatible with SU binding. The choice of amino acids at the library positions was dictated by steric and physicochemical considerations to fit ligand docking into the model ligand pocket. This resulted in a library with (4, 5, 4, 4, 5, 3, 8, 11, 10, 10, 8, 8, 7, 9, 6, 7 and 5) amino acids at the 17 positions, for a total designed library size of 4×10¹³.

The wild type class B TetR from Tn10 was chosen as the starting molecule for generation of shuffling derivatives (SEQ ID NO:2). It is slightly different than the sequence used in computational design (P0ACT4, class D, for which the high-resolution crystal structure 1DU7 is available), but only subtly affects ligand binding.

The starting polynucleotide encoding TetR was synthesized commercially and restriction sites were added for ease of library construction and further manipulations (DNA2.0, Menlo Park, Calif., USA). Added restriction sites include an NcoI site at the 5′ end, a SacI site 5′ of the ligand binding domain (LBD) and an AscI site following the stop codon. Library construction can be localized in a ˜480 bp DNA segment containing the ligand binding region to avoid inadvertent mutations in the other regions, such as the DNA binding domain. The synthetic gene was operably linked downstream of an arabinose inducible promoter, P_{BAD}, using NcoI/AscI to create TetR expression vector pVER7314. The addition of the NcoI site at the 5′ end of the coding region resulted in the insertion of a glycine after the N-terminal methionine at amino acid position one (SEQ ID NO:2). This sequence was used as the wild type TetR control in all assays unless otherwise noted, and observed activity was equivalent to TetR without the serine insertion (SEQ ID NO:1). However, all references to amino acid positions and changes designed and observed use the amino acid numbering of wild type TetR(B) (207 aa) e.g., SEQ ID NO:1.

### B. Library Design

Due to the large number of designed substitutions at many positions in close proximity with one another the computed library (Table 1, Designed Library) was not easily encodable with a small number of degenerate codons. For this reason, the sequence library fabricated and tested in the lab featured the designed amino acid set at 6/17 positions, slightly enlarged at 1/17 positions, and fully degenerate (NNK codon) at 10/17 positions (Table 1). This resulted in much higher predicted sequence diversity, a total of 3×10¹⁹ sequences.

TABLE 1

| | WT | | |
|---|---|---|---|
| Residue | residue | Designed Library | Actual Library |
| 60 | L | A L K M | A L K M |
| 64 | H | A N Q H L | A N Q H L |
| 82 | N | A N S T | A N S T |
| 86 | F | M F W Y | M F W Y |
| 100 | H | H M F W Y | All 20 aa's |
| 104 | R | A R G | A R G |
| 105 | P | A N D G P S T V | All 20 aa's |
| 113 | L | A R N D Q E K M S T V | A R N D Q E K M S T V I P L G H |
| 116 | Q | A R N Q E I K M T V | All 20 aa's |
| 134 | L | A R I L K M F W Y V | All 20 aa's |
| 135 | S | A R N Q H K S T | A R N Q H K S T |
| 138 | G | A H K M F S Y W | All 20 aa's |
| 139 | H | A R Q H L K Y | All 20 aa's |
| 147 | E | A R Q E H L K M Y | All 20 aa's |
| 151 | H | A Q H K I L | All 20 aa's |
| 174 | I | A R Q E L K M | All 20 aa's |
| 177 | F | A R L K M | All 20 aa's |

Library 1 oligonucleotides were designed and assembled by overlap extension (Ness et al. (2002) Nat Biotech 20:1251-1255) to generate a PCR fragment bordered by SacI/AscI restriction sites. Conditions for assembly of all library fragments were as follows: oligonucleotides representing the library are normalized to a concentration of 10 μM and then equal volumes mixed to create a 10 μM pool. PCR amplification of library fragments was performed in six identical 25 μl reactions containing: 1 μM pooled library oligos; 0.5 μM of each rescue primer: L1:5′ and L1:3′ and 200 μM dNTP's in a Herculase II directed reaction (Stratagene, La Jolla, Calif., USA). Conditions for PCR were 98° C. for 1 min (initial denature), followed by 25 cycles of 95° C. denature for 20 seconds, annealing for 45 seconds between 45° C. and 55° C. (gradient), then extending the template for 30 seconds at 72° C. A final extension of 72° C. for 5 minutes completes the reaction. Wild type TetR(B) is excised from the P_{BAD}-tetR expression vector pVER7314 by digestion with SacI/AscI. The pVER7314 backbone fragment is treated with calf intestinal phosphatase and purified, then the fully extended library fragment pool (˜500 bp) digested with SacI/AscI restriction enzymes are inserted to generate the L1 plasmid library. Approximately 50 random clones from library L1 were sequenced and the information compiled for quality control purposes. The results indicated that nearly all amino acids targeted in the diversity set were represented (data not shown). Sequencing revealed that 17% of the sequences contained stop codons. This is less than the predicted 27% (e.g., 10 positions having 1/32 codons be a stop codon, 1−(31/32)¹⁰˜27%). Additionally, sequence analysis showed that 13% of the clones had frame shifts due to mistakes in the overlap extension process. Thus, overall approximately 30% of the library consisted of clones encoding truncated polypeptides.

### C. Screen Set Up

In order to test the library for rare clones reacting to thifensulfuron-methyl (Ts) a sensitive E. coli based genetic screen was developed. The screen is a modification of an established assay system (Wissmann et al. (1991) Genetics 128:225-232). The screen consists of a repressor pre-screen followed by an induction screen. For the repressor prescreen a genetic cascade was developed whereby an nptIII gene encoding kanamycin resistance is under the control of a lac promoter. The lac promoter is repressed by the Lac repressor encoded by lacI, whose expression is in turn controlled by the tet promoter (PtetR). The tet promoter is repressed by TetR which blocks LacI production and thus ultimately enables kanamycin resistance to be expressed.

Since the tet regulon has bivalent promoters, one promoter for tetR and one promoter for tetA, the same strain was engineered with the E. coli lacZ gene encoding enzyme reporter β-galactosidase under control of the tetA promoter (PtetA). The dual regulon encoding both lacI and lacZ was then bordered by strong transcriptional terminators: the E. coli RNA ribosomal operon terminator rrnB T1-T2 (Ghosh et al. (1991) J Mol Biol 222:59-66) and the E. coli RNA polymerase subunit C terminator rpoC, such that spurious transcripts read in the direction of either tet promoter would not interfere with expression of any other transcript. In the presence of functional TetR, the strain exhibits a lac⁻ phenotype and colonies can be easily scored for induction by novel chemistry with X-gal, wherein induction gives increased blue colony color. In addition, induction with novel chemistry in liquid cultures can be measured quantitatively by employing β-galactosidase enzyme assays with either colorimetric or fluorimetric substrates.

In order to obtain better penetration of SU compounds into E. coli (Robert LaRossa—DuPont: personal communication), the host strain tolC locus was knocked out with the incoming Plac-nptIII reporter. A strong transcriptional terminator, T22 from S. typhimurium phage P22, was placed upstream of the lac promoter to prevent unregulated leaky expression of the conditional kanamycin resistance marker. The name of the final engineered strain is E. coli KM3.

The population of shuffled tetR LBD's was cloned into an Ap^{r}/ColE1 based vector pVER7314 behind the P_{BAD} promoter. This was designed to enable fine control of TetR expression by variation of arabinose concentrations in the growth medium (Guzman et al. (1995) J Bacteriol 177:4121-4130). Despite being under the control of the P_{BAD} promoter, TetR protein is expressed at a sufficient level in the absence of added arabinose to enable selection for kanamycin resistance in strain KM3. Nevertheless, expression can be increased by addition of arabinose, for example, if a change in assay stringency is desired.

### D. Library Screening

Following assembly of L1 oligos and capture in vector pVER7314, the resulting library was transformed into E. coli strain KM3 and plated on LB containing 50 μg/ml carbenicillin to select for library plasmids, and 60 μg/ml kanamycin to select for the active repressor population in the absence of target ligand (“apo-repressors”). DNA sequence analysis of this selected population indicated that this step highly enriched several library positions. In addition, this step eliminated clones with premature stop codons and or frame shift mutations. Subsequently, these apo-repressor sequences were screened for alteration in repressor activity in the presence of Harmony® (Ts) by replica plating the Km^{r} pre-selected population from liquid cultures in 384-well format onto M9 agar containing 0.1% glycerol as carbon source, 0.04% casamino acids (to prevent branched chain amino acid starvation caused by sulfonylurea application), 50 μg/ml carbenicillin for plasmid maintenance, 0.004% X-gal to detect β-galactosidase activity, and +/−SU inducer Ts at 20 μg/ml. Initial hits were identified from a population of nearly 20,000 colonies screened for response to Ts following incubation at 30° C. for 2 days. Fourteen putative hits identified were then re-tested under the same conditions but in 96-well format. DNA sequence analysis revealed that clones L1-3 and L1-19 are identical and that the most intensely responding hits (L-2,-3(19), -5, -9, -11 and -20) had significant enrichment at several library positions, indicating an involvement in ligand interaction, directly or indirectly. The same library was then re-screened to identify a further 10 hits to bring the total number of clones to 23.

All 23 putative hits were subsequently screened in the same plate assay format with a panel of nine sulfonylurea (SU) compounds registered for commercial use (Table 2), wherein 11 hits were found to respond significantly to other SU ligands (Table 3). For this experiment, E. coli clones encoding L1 hits or wt TetR (SEQ ID NO:2) were arrayed in 96-well format and stamped onto M9 X-gal assay media with or without test SU compounds at 20 μg/ml. Following 48 hrs growth at 30° C. the plates were digitally imaged and the colony color intensity converted to relative values of β-galactosidase activity. Inducers used: thifensulfuron (Ts), metsulfuron (Ms), sulfometuron (Sm), ethametsulfuron (Es), tribenuron (Tb), chlorimuron (Ci), nicosulfuron (Ns), rimsulfuron (Rs), chlorsulfuron (Cs) at 20 ppm and anhydrotetracycline (atc) as the positive control at 0.4 μM for induction of wt TetR. Some sulfonylurea compounds, particularly chlorimuron, ethametsulfuron, and chlorsulfuron were more potent activators than the starting ligand Harmony®.

TABLE 2
SU Compound

| Common Name | Product | Commercial Use |
|---|---|---|
| Thifensulfuron-methyl (Ts) | Harmony ® | Cereals, corn, soybean |
| Metsulfuron-methyl (Ms) | Ally ® | Cereals, pasture |
| Sulfometuron-methyl (Sm) | Oust ® | Vegetation management |
| Ethametsulfuron-methyl (Es) | Muster ® | Canola |
| Tribenuron-methyl (Tb) | Express ® | Cereal, sunflower |
| Chlorimuron-ethyl (Ci) | Classic ® | Soybean |
| Nicosulfuron (Ns) | Accent ® | Corn |
| Rimsulfuron (Rs) | Matrix ® | Corn, tomato, potato |
| Chlorsulfuron (Cs) | Glean ® | Cereals |

TABLE 3

| | Inducer | | | | | | | | | | |
|---|---|---|---|---|---|---|---|---|---|---|---|
| clone | None | Ts | Ms | Sm | Es | Tb | Ci | Ns | Rs | Cs | atc |
| L1-2 | 1.0 | 1.6 | 1.9 | 4.7 | 5.8 | 1.7 | 13.6 | 1.3 | 1.3 | 4.1 | 1.2 |
| L1-7 | 0.0 | 0.1 | 0.2 | 6.4 | 0.1 | 0.2 | 16.5 | 0.1 | 0.2 | 3.1 | 0.0 |
| L1-9 | 0.3 | 1.1 | 1.2 | 0.6 | 11.8 | 0.4 | 9.8 | 0.3 | 0.4 | 23.6 | 0.3 |
| L1-20 | 1.4 | 2.6 | 12.4 | 6.0 | 15.0 | 2.6 | 13.5 | 1.6 | 2.0 | 22.0 | 2.0 |
| L1-22 | 0.1 | 0.0 | 0.1 | 17.2 | 0.3 | 0.3 | 10.4 | 0.2 | 0.1 | 0.2 | 0.0 |
| L1-24 | 0.1 | 0.3 | 0.4 | 3.1 | 0.2 | 1.6 | 22.1 | 0.3 | 0.3 | 3.3 | 0.1 |
| L1-28 | 0.0 | 0.1 | 18.8 | 1.1 | 0.8 | 0.3 | 14.6 | 0.1 | 0.2 | 5.8 | 0.0 |
| L1-29 | 0.0 | 0.0 | 13.5 | 2.7 | 1.7 | 0.3 | 20.9 | 0.1 | 0.1 | 15.8 | 0.0 |
| L1-31 | 0.3 | 0.9 | 0.5 | 0.9 | 13.7 | 0.1 | 1.1 | 0.5 | 0.4 | 1.4 | 0.4 |
| L1-38 | 9.5 | 16.7 | 14.7 | 18.3 | 14.8 | 15.8 | 15.3 | 8.7 | 9.5 | 14.0 | 6.4 |
| L1-44 | 0.2 | 1.9 | 2.9 | 0.4 | 2.4 | 0.4 | 6.7 | 0.4 | 0.3 | 12.0 | 0.2 |
| TetR | 0.0 | 0.0 | 0.0 | 0.1 | 0.0 | 0.1 | 0.1 | 0.1 | 0.1 | 0.0 | 25.0 |

The initial screenings of library 1 also detected library members having reverse repressor activity (SEQ ID NO:412-419), wherein the polypeptide was bound to the operator in the presence of SU ligand. These hits showed β-galactosidase expression without SU ligand, which was substantially reduced upon addition of the ligand, for example thifensulfuron. These hits were subsequently screened in the same plate assay format as described above with the panel of nine sulfonylurea (SU) compounds registered for commercial use (Table 3), wherein 8 hits were found to respond significantly to other SU ligands (Table 4).

TABLE 4

| | Inducer | | | | | | | | | | |
|---|---|---|---|---|---|---|---|---|---|---|---|
| clone | Blank | Ts | Ms | Sm | Es | Tb | Ci | Ns | Rs | Cs | atc |
| L1-18 | 1.34 | 1.13 | 0.79 | 0.94 | 0.37 | 1.65 | 0.36 | 1.44 | 2.55 | 1.22 | 2.35 |
| L1-21 | 2.88 | 0.79 | 0.89 | 2.39 | 0.61 | 2.13 | 0.07 | 2.74 | 2.31 | 0.89 | 2.81 |
| L1-25 | 1.17 | 0.64 | 0.32 | 0.63 | 0.13 | 1.72 | 0.11 | 1.21 | 1.08 | 0.28 | 1.22 |
| L1-33 | 7.59 | 5.51 | 4.29 | 5.02 | 2.11 | 4.71 | 0.76 | 5.34 | 10.32 | 3.74 | 8.25 |
| L1-34 | 2.37 | 2.97 | 1.47 | 2.00 | 1.33 | 2.26 | 0.43 | 2.91 | 2.30 | 0.85 | 3.68 |
| L1-36 | 1.52 | 0.48 | 0.38 | 0.50 | 0.20 | 0.57 | 0.21 | 1.81 | 1.84 | 0.24 | 1.70 |
| L1-39 | 3.65 | 1.42 | 0.75 | 0.91 | 0.60 | 0.97 | 0.49 | 3.03 | 4.72 | 0.89 | 4.92 |
| L1-41 | 4.05 | 1.46 | 0.56 | 0.67 | 0.18 | 1.41 | 0.39 | 2.75 | 4.05 | 0.61 | 4.21 |
| TetR | 0.00 | 0.08 | 0.08 | 0.23 | 0.06 | 0.13 | 0.18 | 0.18 | 0.20 | 0.15 | 10.45 |

E. Correlation of First Round Shuffling Results with the Structural Model

Significant enrichment occurred at most library positions, where enrichment includes biases favoring particular amino acids and biases disfavoring particular amino acids. The initial screening involved two stages to identify both repressor and de-repressor functions. Enrichment occurred in both stages of screening. In the first stage, positions were enriched by the selection for “apo repressors’, that is, proteins that repress gene transcription in the absence of ligand. In the second stage, positions were enriched by the selection for “activators”, that is, proteins that allow gene transcription in the presence of ligand. Positions may be enriched by either selection criterion, by both criteria, or by neither. The first-round screening results for repressor activity are summarized below:

| | Amino Acid Bias Observed | | |
|---|---|---|---|
| Position | Apo repressor | SU Induction | Both |
| 60 | | L (not K) | |
| 64 | | Q, N (not L, A) | |
| 82 | N (not A, T) | A (not N, S) | |
| 86 | (not M) | M (not W) | |
| 100 | R (not K, Q) | C, W (not H, K, Q) | |
| 104 | G | A | |
| 105 | C, G, L, V (not H, K) | L, W (not G, S) | L |
| 113 | A (not G, P) | A, I (not D, G) | A |
| 116 | (not GL) | M, V (not A, R) | |
| 134 | M, S | I, R, W (not G) | |
| 135 | K, R (not H, S) | Q, R (not A, T) | R |
| 138 | (not T) | A, C, R, V (not L, P, Q, T) | |
| 139 | R (not H) | T (not L, P) | |
| 147 | (not A, C) | R, W (not A, S) | |
| 151 | R (not C, G, Q) | M, R (not V) | R |
| 174 | V (not L, R) | W (not F, L) | |
| 177 | T (not S) | K, L (not P, T) | |

Several rounds of library design and shuffling were completed. Resulting polynucleotides and encoded SuRs are provided in the Sequence Listing.

### Summary

FIG. 1 provides a cumulative summary of the introduced diversity and observed amino acids in active SuRs obtained from the screening assays. Even though some positions were strongly biased (i.e. observed more frequently in the selected population) as indicated by larger bolded type, the entirety of introduced diversity was observed in the full hit populations.

### Example 2

### Sulfonylurea Repressor Ligand Binding Domain Fusions

The ligand binding domains from the sulfonylurea repressors provided herein can be fused to alternative DNA binding domains in order to create further sulfonylurea repressors that selectively and specifically bind to other DNA sequences (e.g., Wharton & Ptashne (1985) Nature 316:601-605). Many domain swapping experiments have been published, demonstrating the breadth and flexibility of this approach. Generally, an operator binding domain or specific amino acid/operator contact residues from a different repressor system will be used, but other DNA binding domains can also be used. For example, a polynucleotide encoding a TetR(D)/SuR chimeric polypeptide consisting of the DNA binding domain from TetR(D) (e.g., amino acid residues 1-50) and ligand binding domain of a SuR residues (e.g., amino acid residues 51-208 from TetR(B) can be constructed using any standard molecular biology method or combination thereof, including restriction enzyme digestion and ligation, PCR, synthetic oligonucleotides, mutagenesis or recombinational cloning. For example, a polynucleotide encoding a SuR comprising a TetR(D)/SuR chimera can be constructed by PCR (Landt et al. (1990) Gene 96:125-128; Schnappinger et al. (1998) EMBO J 17:535-543) and cloned into a suitable expression cassette and vector. Any other TetOp binding domains can be substituted to produce a SuR that specifically binds to the cognate tet operator sequence.

In addition, mutant TetO^{c} binding domains from variant TetR's having suppressor activity on constitutive operator sequences (tetO-4C and tetO-6C) can be used (see, e.g., Helbl & Hillen (1998) J Mol Biol 276:313-318; and Helbl et al. (1998) J Mol Biol 276:319-324). Further, the polynucleotides encoding these DNA binding domains can be modified to change their operator binding properties. For example, the polynucleotides can be shuffled to enhance the binding strength or specificity to a wild type or modified tet operator sequence, or to select for specific binding to a new operator sequence.

Additional variants could be made by fusing a SuR repressor, or a SuR ligand binding domain to an activation domain. Such systems have been developed using Tet repressors. For example, one system converted a tet repressor to an activator via fusion of the repressor to a transcriptional transactivation domain such as herpes simplex virus VP16 and the tet repressor (tTA, Gossen & Bujard (1992) PNAS 89:5547-5551). The repressor fusion is used in conjunction with a minimal promoter which is activated in the absence of tetracycline by binding of tTA to tet operator sequences. Tetracycline inactivates the transactivator and inhibits transcription.

### Example 3

### Operator Binding

To confirm that sulfonylurea ligands were binding directly to the modified repressor molecules and causing derepression, an in vitro tet operator gel shift study was undertaken.

An electrophoretic gel mobility shift assay (EMSA) of EsR variants was done to monitor binding to the tet operator (tetO) sequence and response of the complex to inducers Es and Cs. TetO consists of a synthetic 48 bp tetO-containing fragment created from hybridization of oligonucleotide tetO1 (SEQ ID NO:837): 5′-CCTAATTTTTGTTGACACTCTATCATTGATAGAGTTATTTTACCACTC-3′ and complementary oligonucleotide tetO2 (SEQ ID NO:838): 5′-GGATTAAAAACAACTGTGAGATAGTAACTATCTCAATAAAATGGTGAG-3′ The tet operator is shown in bold.

An oligonucleotide and its complement of the same size containing no palindromic sequence was used as a control (SEQ ID NO:839): 5′-CCTAATTTTTGTTGACTGTGTTAGTCCATAGCTGGTATTTTACCACTC-3′ and complementary oligonucleotide (SEQ ID NO:840): 5′-GGATTAAAAACAACTGACACAATCAGGTATCGACCATAAAATGGTGAG-3′

Five pmol of TetO or control DNA was mixed with the indicated amounts of ethametsulfuron repressor protein (L7A11, SEQ ID NO:409) or BSA control with or without inducer in complex buffer containing 20 mM Tris-HCl (pH8.0) and 10 mM EDTA. The mixture was incubated at room temperature for 0.5 hour before loading onto the gel. The reaction was electrophoresed on a Novex 6% DNA retardation gel (Invitrogen) at room temperature, 38 V in 0.5× TBE buffer for about 2 hours. DNA was detected by ethidium bromide staining. These results (not shown) demonstrated that the modified repressors bind to operator DNA and are released from the operator sequence in an inducer-specific and dose dependent manner.

### Example 4

### Binding and Dissociation Constants

Select SU repressors were further characterized for operator and ligand binding, affinity and dissociation kinetics using Biacore™ SPR technology (Biacore, GE Healthcare, USA). The technology is based on surface plasmon resonance (SPR), an optical phenomenon that enables detection of unlabeled interactants in real time. The SPR-based biosensors can be used in determination of active concentration, screening and characterization in terms of both affinity and kinetics.

The kinetics of an interaction, i.e., the rates of complex formation (kₐ) and dissociation (k_{d}), can be determined from the information in a sensorgram. If binding occurs as sample passes over a prepared sensor surface, the response in the sensorgram increases. If equilibrium is reached, a constant signal is seen. Replacing the sample with buffer causes the bound molecules to dissociate and the response decreases. Biacore evaluation software generates the values of kₐ and k_{d} by fitting the data to interaction models.

The affinity of an interaction is determined from the level of binding at equilibrium (seen as a constant signal) as a function of sample concentration. Affinity can also be determined from kinetic measurements. For a simple 1:1 interaction, the equilibrium constant K_{D} is the ratio of the kinetic rate constants, k_{d}/kₐ.

### A. Operator Binding Characterization of Repressors

| Repressor | kₐ (M⁻¹ s⁻¹) | K_{d} (s⁻¹) | K_{D} (nM) |
|---|---|---|---|
| Wt TetR | 3.3 × 10⁵ | 3.0 × 10⁻³ | 9.0 ± 1.0 |
| L7-1C03-A5 | 4.7 × 10⁴ | 7.8 × 10⁻³ | 150 ± 5 |
| L7-3E03-D1 | 5.5 × 10⁴ | 1.1 × 10⁻² | 200 ± 50 |
| L7-1F08-A11 | 7.1 × 10⁴ | 1.7 × 10⁻² | 250 ± 120 |
| L7-1G06-B2 | 4.6 × 10⁴ | 1.9 × 10⁻² | 430 ± 160 |

### B. SU Binding Characterization of Repressors

KD (μM)

| Repressor | Es +Mg | Es −Mg | Cs +Mg | Cs −Mg | ATC +Mg |
|---|---|---|---|---|---|
| L7-1C03-A5 | 0.46 | 1.78 | 83 | 365 | Null |
| L7-1F08-A11 | 0.45 | 1.09 | 40 | 92 | Null |
| L7-1G06-B2 | 0.53 | 2.15 | 60 | 255 | Null |
| L7-3E03-D1 | 0.73 | 2.15 | 48 | 115 | Null |
| Wt TetR | Null | Null | Null | Null | 0.0036 |

### Example 5

### Plant Assays

A. Nicotiana benthamiana Leaf Infiltration Assay

An in planta transient assay system was developed to rapidly confirm functionality of candidate SU-responsive chemical switch systems in planta prior to testing in transgenic plants. An Agrobacterium based leaf infiltration assay was developed to measure repression and derepression activities. N. benthamiana leaves were infiltrated with a mixture of reporter and effector (repressor) Agrobacterium strains such that reporter activity is reduced by ˜90% in the presence of the effector and then derepressed following treatment with inducer.

Two ethametsulfuron repressors, EsR A11 and EsR D01, were selected for testing dose response to ethametsulfuron in conjunction with a wild type TetR control. Three test strains were derived by transformation of A. tumefaciens EHA105 with three different T-DNA based vectors. Agrobacterium strains harboring binary vectors with a 35S::tetO-Renilla Luciferase reporter and dPCSV-tetR or -SuR effector variants were constructed. In addition to these tester cultures, an existing Agrobacterium strain harboring a dMMV-GFP T-DNA was added to the assay mixture to monitor the progression of Agrobacterium infection for sampling purposes.

To test the system for chemical switch activation, mixtures of tester Agrobacterium cultures containing 10% 35S::tetO-ReLuc reporter Agro, 10% dMMV-GFP Agro and 80% dPCSV-wt tetR Agro were infiltrated into N. benthamiana leaves and co-cultivated for 36 hours in the growth chamber. Infiltrated leaves were then excised and the petiole placed into water (negative control) or inducer at the test concentrations and allowed to co-cultivate for another 36 hours. Infected leaf areas were assayed for Renilla luciferase activity and inducer treatments compared. The results show significant repression of reporter activity (˜90%) with no inducer treatment (water control) for all tested repressors, and significant but incomplete induction of the EsR D01 repressor at inducer concentration as low as 0.02 ppm Es. Both EsR's were fully induced at 0.2 ppm Es whereas TetR was only fully induced at 2.0 ppm anhydrotetracycline.

B. N. tabacum BY-2 Cell Chemical Switch Assay

In addition to the leaf assay it was desired to have an in planta assay to enable high throughput screening. A system similar to the leaf assay was designed using tobacco BY-2 cell culture in 96-well format. BY-2 cell culture was transformed with a dMMV-HRA construct such that the culture would withstand treatment with target sulfonylurea test compounds. The resultant cell line grows and is fully resistant to 200 ppb chlorsulfuron.

### C. Sulfonylurea-Responsive Chemical Switch in Soybean

Any transformation protocols, culture techniques, soybean source, and media, and molecular cloning techniques can be used with the compositions and methods.

i. Transformation and Regeneration of Soybean (Glycine max)

Transgenic soybean lines are generated by particle gun bombardment (Klein et al. Nature 327:70-73 (1987); U.S. Pat. No. 4,945,050) using a BIORAD Biolistic PDS1000/He instrument and either plasmid or fragment DNA. The following stock solutions and media are used for transformation and regeneration of soybean plants:

Stock solutions:
Sulfate 100× Stock: 37.0 g MgSO4.7H2O, 1.69 g MnSO4.H2O, 0.86 g ZnSO4.7H2O, 0.0025 g CuSO4.5H2O
Halides 100× Stock: 30.0 g CaCl2.2H2O, 0.083 g KI, 0.0025 g CoCl2.6H2O
P, B, Mo 100× Stock: 18.5 g KH2PO4, 0.62 g H3BO3, 0.025 g Na2MoO4.2H2O
Fe EDTA 100× Stock: 3.724 g Na2EDTA, 2.784 g FeSO4.7H2O
2,4-D Stock: 10 mg/mL 2,4-Dichlorophenoxyacetic acid
B5 vitamins, 1000× Stock: 100.0 g myo-inositol, 1.0 g nicotinic acid, 1.0 g pyridoxine HCl, 10 g thiamine HCL.

Media (per Liter):
SB199 Solid Medium: 1 package MS salts (Gibco/BRL, Cat. No. 11117-066), 1 mL B5 vitamins 1000× stock, 30g Sucrose, 4 ml 2,4-D (40 mg/L final concentration), pH 7.0, 2 g Gelrite
SB1 Solid Medium: 1 package MS salts (Gibco/BRL, Cat. No. 11117-066), 1 mL B5 vitamins 1000× stock, 31.5 g Glucose, 2 mL 2,4-D (20 mg/L final concentration), pH 5.7, 8 g TC agar
SB196: 10 mL of each of the above stock solutions 1-4, 1 mL B5 Vitamin stock, 0.463 g (NH4)2 SO4, 2.83 g KNO3, 1 mL 2,4 D stock, 1 g asparagine, 10 g sucrose, pH 5.7
SB71-4: Gamborg's B5 salts, 20 g sucrose, 5 g TC agar, pH 5.7.
SB103: 1 pk. Murashige & Skoog salts mixture, 1 mL B5 Vitamin stock, 750 mg MgCl2 hexahydrate, 60 g maltose, 2 g gelrite, pH 5.7.
SB166: SB103 supplemented with 5 g per liter activated charcoal.

Soybean embryogenic suspension cultures are initiated twice each month with 5-7 days between each initiation. Pods with immature seeds from available soybean plants 45-55 days after planting are picked, removed from their shells and placed into a sterilized magenta box. The soybean seeds are sterilized by shaking them for 15 min in a 5% v/v CLOROX™ solution with 1 drop of ivory soap. Seeds are rinsed using 2 1-liter bottles of sterile distilled water and those less than 3 mm are placed on individual microscope slides. The small end of the seed is cut and the cotyledons pressed out of the seed coat. Cotyledons are transferred to plates containing SB199 medium (25-30 cotyledons per plate) for 2 weeks, then transferred to SB1 for 2-4 weeks. Plates are wrapped with fiber tape. After this time, secondary embryos are cut and placed into SB196 liquid media for 7 days.

Soybean embryogenic suspension cultures (cv. Jack) are maintained in 50 mL liquid medium SB196 on a rotary shaker, 150 rpm, 26° C. with cool white fluorescent lights on 16:8 h day/night photoperiod at light intensity of 60-85 μE/m2/s. Cultures are subcultured every 7 days to two weeks by inoculating approximately 35 mg of tissue into 50 mL of fresh liquid SB196 (the preferred subculture interval is every 7 days).

### Preparation of DNA for Bombardment:

Intact plasmid DNA or DNA fragments containing only the recombinant DNA expression cassette(s) of interest can be used in particle gun bombardment procedures. For every seventeen bombardment transformations, 85 μL of suspension is prepared containing 1 to 90 picograms (pg) of plasmid DNA per base pair of each DNA plasmid. Both recombinant DNA plasmids are co-precipitated onto gold particles as follows. The DNAs in suspension are added to 50 μL of a 10-60 mg/mL 0.6 μm gold particle suspension and then combined with 50 μL CaCl2 (2.5 M) and 20 μL spermidine (0.1 M). The mixture is vortexed for 5 sec, spun in a microfuge for 5 sec, and the supernatant removed. The DNA coated particles are then washed once with 150 μL of 100% ethanol, vortexed and pelleted, then resuspended in 85 μL of anhydrous ethanol. Five μL of the DNA coated gold particles are then loaded on each macrocarrier disk.

Approximately 150 to 250 mg of two-week-old suspension culture is placed in an empty 60 mm×15 mm Petri plate and the residual liquid removed from the tissue using a pipette. The tissue is placed about 3.5 inches away from the retaining screen and each plate of tissue is bombarded once. Membrane rupture pressure is set at 650 psi and the chamber is evacuated to −28 inches of Hg.

After bombardment, tissue from each bombarded plate is divided and placed into two flasks of SB196 liquid culture maintenance medium per plate of bombarded tissue. Seven days post bombardment, the liquid medium in each flask is replaced with fresh SB196 culture maintenance medium supplemented with 100 ng/ml selective agent (selection medium). Transformed soybean cells can be selected using a sulfonylurea (SU) compound such as 2 chloro N((4 methoxy 6 methy 1,3,5 triazine 2 yl)aminocarbonyl)benzenesulfonamide (common names: DPX-W4189 and chlorsulfuron). Chlorsulfuron (Cs) is the active ingredient in the DuPont sulfonylurea herbicide, GLEAN®. The selection medium containing SU is replaced every two weeks for 6-8 weeks. After the 6-8 week selection period, islands of green, transformed tissue are observed growing from untransformed, necrotic embryogenic clusters. These putative transgenic events are isolated and kept in SB196 liquid medium with Cs at 100 ng/ml for another 2-6 weeks with media changes every 1-2 weeks to generate new, clonally propagated, transformed embryogenic suspension cultures. Embryos spend a total of around 8-12 weeks in contact with Cs. Suspension cultures are subcultured and maintained as clusters of immature embryos and also regenerated into whole plants by maturation and germination of individual somatic embryos.

Somatic embryos became suitable for germination after four weeks on maturation medium (1 week on SB166 followed by 3 weeks on SB103). They are then removed from the maturation medium and dried in empty Petri dishes for up to seven days. The dried embryos are then planted in SB71 4 medium where they are allowed to germinate under the same light and temperature conditions as described above. Germinated embryos are transferred to potting medium and grown to maturity for seed production.

ii. Vector Construction and Testing

Plasmids were made using standard procedures and from these plasmids DNA fragments were isolated using restriction endonucleases and agarose gel purification. Each DNA fragment contained three cassettes. Cassette 1 is a reporter expression cassette; Cassette 2 is the repressor expression cassette; and, Cassette 3 is an expression cassette providing an HRA gene. The repressors tested in Cassette 2 are described in Table 5. The polynucleotides comprising the repressor coding region were synthesized to comprise plant preferred codons. In all cases Cassette 1 contained a 35S cauliflower mosaic virus promoter having three tet operators introduced near the TATA box (Gatz et al. (1992) Plant J 2:397-404 (3XOpT 35S)) driving expression of DsRed followed by the 35S cauliflower mosaic virus 3′ terminator region. In all cases cassette three contained the S-adenosylmethionine synthase promoter followed by the HRA version of the acetolactose synthase (ALS) gene followed by the Glycine max ALS 3′ terminator. The HRA version of the ALS gene confers resistance to sulfonylurea herbicides. EF1A1 is the promoter of a soybean translation elongation factor EF1 alpha described in US2008/0313776.

TABLE 5

| Fragment | Fragment | | Repressor | Repressor | Fragment |
|---|---|---|---|---|---|
| Name | alias | Cassette 2 | alias | SEQ ID | SEQ ID |
| PHP37586A | CHSW004 | EF1A1::EsR1::Nos3′ | L7-IC3-A5 | 408 | 841 |
| PHP37587A | CHSW005 | EF1A1::EsR2::Nos3′ | L7-1F8-A11 | 409 | 842 |
| PHP37588A | CHSW006 | EF1A1::EsR2::Nos3 | L7-1G6-B2 | 410 | 843 |
| PHP37589A | CHSW007 | EF1A1::EsR4::Nos3′ | L7-3E3-D1 | 411 | 844 |
| PHP39389A | CHSW010 | EF1A1::EsR5::CaMV35S3′ | L12-1-10 | 406 | 845 |
| PHP39390A | CHSW011 | EF1A1::EsR6::CaMV35S3′ | L13-2-23 | 407 | 846 |

DNA fragments were used for soybean transformation as described above. Plants were regenerated and leaf discs (˜0.5 cm) were harvested from young leaves. The leaf discs were incubated in SB103 liquid media containing 0 ppm, 0.5 ppm or 5 ppm ethametsulfuron for 2-5 days. Ethametsulfuron (product number PS-2183) was purchased from Chem Service (West Chester, Pa.) and solubilized in either 10 mM NaOH or 10 mM NH₄OH. On each day leaf discs were examined under a dissecting microscope with a DsRed band pass filter. The presence of DsRed was scored visually.

Plants that expressed DsRed at 0 time were scored as leaky. Plants that did not express DsRed after five days were scored as negative. Plants that expressed DsRed after addition of ethametsulfuron were scored as inducible. Results from the experiments are shown in Table 6.

TABLE 6

| | | Total | % | % | % |
|---|---|---|---|---|---|
| Name | Alias | Events | Leaky | Negative | Inducible |
| PHP37586A | CHSW004 | 12 | 33 | 33 | 33 |
| PHP37587A | CHSW005 | 28 | 7 | 50 | 43 |
| PHP37588A | CHSW006 | 6 | 0 | 0 | 100 |
| PHP37589A | CHSW007 | 9 | 0 | 22 | 78 |
| PHP39389A | CHSW010* | 19 | 5 | 26 | 42 |
| PHP39390A | CHSW011* | 35 | 0 | 17 | 57 |

*In these cases the total does not equal 100% as multiple plants were examined from some events and, in some cases, different plants from the same event behaved differently.

The repressor proteins respond to ethametsulfuron as evidenced by induction of DsRed expression. Plants derived from the first four fragments showed visual evidence of DsRed after three days of incubation. Plants derived from the last two fragments showed visual evidence of DsRed after two days of incubation. The presence of DsRed was scored visually, but this was confirmed by Western Blot analysis on a selection of transformants using a rabbit polyclonal antibody (ab41336) from Abcam (Cambridge, Mass.).

Leaf punches were harvested as described above from a selection of transformants and incubated in SB103 media with 0, 5, 50, 250 and 500 ppb ethametsulfuron. At all concentrations of ethametsulfuron, leaves showed visual evidence of DsRed after three days of incubation. At the lowest concentration (5 ppb) the presence of DsRed was limited to a “halo” near the outside edge of the leaf disc.

Plants were allowed to mature. Since soybeans are self fertilizing, the T1 seeds derived from these plants would be expected to segregate 1 wild type:2 hemizygote:1 homozygote if only one transgene locus was created during transformation. Sixteen seeds from five different events were planted and allowed to germinate. Leaf punches were collected from young seedlings and incubated in SB103 media with 0 and 5 ppm ethametsulfuron. Leaf discs were scored for DsRed expression and 0 and 3 days and results are shown in Table 7.

TABLE 7

| | | Total | # | # | # |
|---|---|---|---|---|---|
| | | # Seeds | Leaky | Negative | Inducible |
| Name | Event ID | Germinated | Plants | Plants | Plants |
| PHP37586A | 6048.3.8.3 | 11 | 0 | 2 | 9 |
| PHP37587A | 6049.2.2.4 | 12 | 0 | 5 | 7 |
| PHP37588A | 6150.3.2.1 | 14 | 0 | 1 | 13 |
| PHP37589A | 6154.4.5.1 | 15 | 0 | 15 | 0 |
| PHP39389A | 6151.4.18.1 | 12 | 3 | 9 | 0 |

### D. Sulfonylurea-Responsive Chemical Switch in Corn

To evaluate SU-responsive chemical switch systems in plants, RFP reporter constructs were constructed and transformed into maize cells via Agrobacterium using the following T-DNA configuration:
RB-35S/TripleOp/Pro::RFP-Ubi Pro::EsR-HRA cassette-PAT cassette-LB.

Using standard molecular biology and cloning techniques, T-DNA vectors having the configuration above comprising selected round 3 SU repressors (EsRs) were constructed. The polynucleotides comprising the repressor coding region were synthesized to comprise plant preferred codons. The constructs are summarized below:

| | SU repressor | SU repressor |
|---|---|---|
| Construct ID | alias (EsR) | SEQ ID |
| PHP37707 | L7-1C3-A5 | 408 |
| PHP37708 | L7-1F8-A11 | 409 |
| PHP37709 | L7-1G6-B2 | 410 |
| PHP37710 | L7-3E3-D1 | 411 |

The reporter construct T-DNA contained a CaMV35S promoter with two tet operators flanking the TATA box and one downstream adjacent to the transcription start site (Gatz et al. (1992) Plant J 2:397-404) driving expression of the red fluorescent protein gene, a ubiquitin driven SU repressor (EsR), an expression cassette containing the maize HRA gene for SU resistance and a moPAT expression cassette for selection.

Immature embryos were transformed using standard methods and media. Briefly, immature embryos were isolated from maize and contacted with a suspension of Agrobacterium, to transfer the T-DNA's containing the sulfonylurea expression cassette to at least one cell of at least one of the immature embryos. The immature embryos were immersed in an Agrobacterium suspension for the initiation of inoculation and cultured for seven days. The embryos were then transferred to culture medium containing carbinicillin to kill off any remaining Agrobacterium. Next, inoculated embryos were cultured on medium containing both carbenicillin and bialaphos (a selective agent) and growing transformed callus was recovered. The callus was then regenerated into plantlets on solid media before transferring to soil to produce mature plants. Approximately 10 single copy events from each of the constructs were sent to the greenhouse.

To evaluate de-repression, callus was transferred to medium with and without ethametsulfuron and chlorsulfuron and RFP fluorescence was observed under the microscope (not shown). Most events de-repressed and there were no obvious differences between the round three repressors tested. To evaluate de-repression in plants, seeds for single copy plants were germinated in the presence of ethamethsulfuron and fluorescence was observed and photographed. As a positive control, a vector containing the same configuration of expression cassettes as PHP37707-10, but with UBI::TetR in place of UBI::EsR, were transformed into maize immature embryos and tested for induction on doxycycline. When grown in the presence of 1 mg/l doxycycline, transgenic callus and plants containing the TetR expression cassette induced over a similar 5-6 day period.

### E. Sulfonylurea-Responsive Chemical Switch in Rice

Mature seed of rice were surface sterilized and placed on callus induction medium (Chu (N6) salts, Eriksson's vitamins, 0.5 mg/L thiamine, 2 mg/L 2,4-D, 2.1 g/L proline, 30 g/L sucrose, 300 mg/L casein hydrolysate, and 100 mg/L myo-inositol, adjusted to pH 5.8). After one week, callus was transformed using Agrobacterium LBA4404, delivering the following T-DNA:
RB-35S PRO:3xTetOp:dsRED::pinII+35S PRO::Adh intron::ESR(L13-2-23)::UBI TERM+Sb-ALS PRO::HRA::pinII-LB

Transgenic events were visually selected as RFP+ calli growing on 100 PPB ethametsulfuron. After herbicide-resistant, red calli were well established, the calli were transferred onto culture medium without ethametsulfuron, and the calli that grew out on this medium did not exhibit red fluorescence (i.e. repression had been re-established). Non-fluorescing events were then transferred to plates of medium containing varying amounts of sulfonylureas (SU). For the control, there was no SU, with additional treatments containing 100 or 500 ppb chlorsulfuron, and 100 or 500 ppb ethametsulfuron, After 20 hours of the induction treatment, micrographs were taken at the same exposure and scored (see Example 7 for scoring criteria) as shown in the table below

| Treatment | RFP Score |
|---|---|
| Control (no SU) | 0 |
| 100 ppb chlorsulfuron | 0-1 |
| 100 ppb ethametsulfuron | 1-2 |
| 500 ppb chlorsulfuron | 2-3 |
| 500 ppb ethametsulfuron | 4 |

These results clearly demonstrated that in the absence of ligand, expression of RFP in rice callus was effectively repressed, and after addition of ligand, varying degrees of de-repression occurred resulting in RFP fluorescence. Ethametsulfuron induced to a greater level than chlorsulfuron, and the 500 ppb treatment for both ligands induced to higher levels than the 100 ppb treatment.

### Example 6

### Controlled Expression Switch Systems

Any transformation protocols, culture techniques, plant, explant, seed, or tissue source, media, construct elements, molecular cloning techniques and diagnostic methods can be used with the compositions and methods.

Several construct elements are used, as indicated by common abbreviations. For convenience, these elements are described briefly. One of skill in the art is able to select alternative elements that provide similar functions and/or characteristics. In some examples, fluorescent reporters are used such as DsRED, AmCYAN, ZsGREEN, and ZsYELLOW, all of which are available from Clontech (Mountain View, Calif.). Elements from CaMV are used, including the promoter (35S Pro, 35SCaMV), an enhancer (35S Enh), and/or terminator (35S 3′, CaMV35S 3′, 35S term). Some cassettes include introns, such as an alcohol dehydrogenase intron (Adh1 intron) from maize. Various terminators are used including terminators from ubiquitin genes (ubi 3′, ubi term), nopaline synthase terminator from Agrobacterium (nos term, nos 3′), proteinase inhibitor protein terminator from potato (pinII 3′, pinII term), or acetolactose synthase (ALS) terminator from soybean (GmALS 3′, ALS term). Besides those already described, promoters include an ALS promoter from S. bicolor (SbALS pro). Coding regions include acetolactose synthase (ALS) variants that provide SU resistance (HRA), developmental genes such as ovule development protein (ODP2) (see, e.g, U.S. Pat. No. 7,579,529), recombinases such as FLP or Cre having modified codon usage (moFLP, moCre) (see, e.g., U.S. Pat. No. 6,720,475, U.S. Pat. No. 6,262,341). Other elements include recombination sites such as FRT sites, or lox sites, wherein FRT1 refers to a wild type minimal FRT site, loxP refers to a wild type minimal lox site, and other nomenclatures refer to non-wild type minimal sites. The abbreviations RB and LB refer respectively to right border and left border sequences from an Agrobacterium T-DNA.

### A. Ethametsulfuron-Inducible Callus Initiation in Maize

Immature maize embryos from maize inbred PHN46 were transformed as describe in Example 5D using Agrobacterium comprising the following:
RB-BSV(AY) PRO::tetOp::Adh1 Intron::ODP2::pinII+35S ENH::ALS PRO::HRA::pinII+Ubi Pro::ESR(3E3)::pinII-LB.

Events were selected on 100 μg/l chlorsulfuron, and plantlets were regenerated in the absence of the SU compound. When the plantlets were approximately 20 cm tall, the leaves were cut into approximately 2-4 mm cross-sections and placed on embryogenic culture medium+/−100 mg/L chlorsulfuron. Over the next two weeks, leaf pieces on the control medium (no SU) became necrotic and died, but leaf pieces on the chlorsulfuron-containing medium were producing callus from the leaf segments.

### B. Ethametsulfuron-induced Recombination in Maize

### 1. Excision

Expression of a polynucleotide of interest may be controlled by inducing excision of an intervening fragment to produce or improve a functional linkage to expression control elements.

Maize immature embryos and mature embryo-derived rice callus were transformed as described above. Each was co-transformed with Agrobacterium LBA4404 containing the following two T-DNAs, each on a separate plasmid:
RB-35S PRO:tetOp::Adh intron::moCRE::pinII+35S PRO::Adh intron::ESR (L13-2-23)::Ubi14 TERM+UBI PRO::Ubi intron::moPAT::pinII-LB
RB-Ubi Pro::SbALS::pinII+Ubi Pro:loxP:AmCYAN::pinII−loxP:ZsYELLOW::pinII-LB

After Agrobacterium transformation, transgenic events were selected on media containing 3 mg/L bialaphos. Herbicide-resistant, blue fluorescent co-transformed calli were recovered and tested for SU-inducible excision. Calli were split onto media+/−250 μg/L ethametsulfuron. For both rice and maize, after one week the calli in the control (no SU) continued to express only the AmCYAN (blue fluorescence), while calli grown in the presence of 250 μg/L ethametsulfuron, yellow fluorescent sectors were observed, indicating excision of AmCYAN and activation of ZsYELLOW expression.

### 2. Inversion

Recombination sites with unequal activity between forward and reverse reactions can be used to stably trigger a chemical switch. Examples of such recombination sites are available, for example see Albert et al. (1995) Plant J 7:649-659 (herein incorporated by reference).

Maize immature embryos are co-transformed with Agrobacterium LBA4404 containing the following 2 T-DNAs, each on a separate plasmid:
RB-35S PRO:tetOp::Adh intron::moCRE::pinII+35S PRO::Adh intron::ESR (L13-2-23)::Ubi14 TERM+UBI PRO::Ubi intron::moPAT::pinI-LB
RB-Ubi Pro::SbALS::pinII+Ubi Pro:lox66:AmCYAN::pinII+pinII (Reverse)::ZsYELLOW (Reverse)::lox71(Reverse)-LB

After transformation, transgenic events are selected on media containing 3 mg/L bialaphos. Herbicide-resistant, blue fluorescent co-transformed calli are recovered and tested for SU-inducible inversion. Calli are split onto medium+/−250 μg/L ethametsulfuron. After one week, the calli on control media (no SU) should continue to express only the AmCYAN (blue fluorescence), while in calli grown in the presence of 250 μg/L ethametsulfuron, should having sectors exhibiting only yellow fluorescent, indicating inversion and activation of ZsYELLOW expression.

### 3. Site-Specific Integration

Maize immature embryos are transformed with Agrobacterium LBA4404 containing the following FLP construct:
RB-35S PRO:tetOp::Adh intron::moFLP::pinII+35S PRO::Adh intron::ESR (L13-2-23)::Ubi14 TERM+Ubi Pro::SbALS::pinII+loxP-Rab17PRO::moCRE::pinII+UBI PRO::Ubi intron::moPAT::pinII−loxP-LB

Callus events are selected on standard maize embryogenic medium+3 mg/L bialaphos for 8 weeks, at which point the calli are transferred onto dry filter paper for 2-3 days to induce Cre recombinase expression and excision of both the Cre and moPAT expression cassettes from the transgenic locus. After desiccation-induced excision of the selectable marker, the callus is moved onto maturation and regeneration media without bialaphos. Regenerated plants are self-pollinated and progeny are analyzed to recover homozygous transgenic events.

T1 progeny containing the two copies of the FLP construct locus are crossed to plants that are homozygous for the SSI target:
RB-Ubi Pro:FRT1:AmCYAN::pinII+Ubi Pro:GAT::pinII-FRT6-LB.

The resultant progeny contain one copy of the inducible FLP recombinase and one copy of the target site. Immature embryos are isolated when they are 1.2 mm in length, and cultured for 3 days on 250 ppb ethametsulfuron. After 3 days of induced moFLP expression, the embryos are moved to high osmotic medium (560M) and bombarded with the donor vector containing FRT1::moPAT::pinII+Ubi Pro:YFP::pinII-FRT6.

After bombardment, embryos are cultured for an additional week on 560P medium+250 ppb ethametsulfuron with no herbicide selection. Upon introduction, FLP recombinase facilitates the replacement of CFP::pinII+Ubi Pro:GAT::pinII with moPAT::pinII+Ubi Pro:YFP::pinII, changing the callus phenotype from {CFP+,GAT+} to {YFP+,PAT+}. One week after bombardment, embryos are removed from the SU medium and placed on medium+3 mg/L bialaphos. Bialaphos-resistant, yellow fluorescent site-specific integration events are recovered and fertile maize plants regenerated.

### C. Inducible Gene Silencing in Soybean

Any transformation protocols, culture techniques, soybean source, and media, and molecular cloning techniques can be used with the compositions and methods.

Plasmids are made using standard procedures and from these plasmids DNA fragments are isolated using restriction endonucleases and agarose gel purification. Each DNA fragment will contain five cassettes:
Cassette 1 contains sequence encoding a maize optimized Cre recombinase (see, e.g., U.S. Pat. No. 6,262,341, herein incorporated by reference) under the control of a 35S cauliflower mosaic virus promoter having three tet operators introduced near the TATA box (Gatz et al. (1992) Plant J 2:397-404 (3XOpT 35S));
Cassette 2 contains the sequence encoding a silencing construct, in this case an artificial miRNA comprising a soybean microRNA159 backbone and a FAD2-1b miRNA (see US2009/0155910, herein incorporated by reference) under the control of a mirabilis mosaic virus (MMV) promoter. Cassette 3 is inserted between the promoter and the silencing construct of Cassette 2;
Cassette 3 comprises a hygromycin resistance gene under the control of a 35S cauliflower mosaic virus promoter and flanked by LOXP sites;
Cassette 4 is the repressor expression cassette; and,
Cassette 5 is an expression cassette providing an HRA gene.
Cassette 4 and Cassette 5 are equivalent to Cassette 2 and Cassette 3 respectively and described in Example 5C. The sequence of the entire DNA fragment is given in SEQ ID NO:847.

DNA fragments will be used for soybean transformation as described in Example 5C above. Plants will be regenerated and seeds collected. These seeds will be treated with ethametsulfuron and planted. T2 seeds will be collected and assayed for fatty acid levels using standard GC-mass spectrometry methods. It is expected that the treatment with ethametsulfuron will cause induction of the Cre recombinase which will excise cassette 3. This then allows the expression of cassette 2 and the silencing of the gene of interest. In this case the gene of interest is the fatty acid desaturase 2-1 and silencing causes an increase in the amount of oleic acid (18:1) that accumulates in the seed.

It is understood by those well versed in the art that the 159-FAD2-1b artificial microRNA can be substituted by any polynucleotide of interest that directs gene silencing. This includes but is not limited to artificial microRNAs, RNAi constructs, siRNA constructs, sense silencing constructs, antisense silencing constructs, constructs that cause the production of double stranded-RNA, ribozymes, and engineered RnaseP constructs. Furthermore the promoter driving cassette 2, or another cassette, can be constitutive (as shown here) or can be a tissue-preferred, a developmental stage-preferred promoter, an inducible promoter or a repressible promoter. For example, an embryo-preferred promoter such as a soybean conglycinin promoter could be used in cassette 2.

### Example 7

### Plant Promoters Containing Tet Operators

Several plant promoters were evaluated and engineered to contain tet operator sequences. Generally, three copies of a tet operator sequence (SEQ ID NO:848) were placed into the promoter. The placement of the operator sequences was essentially modeled based on the 355-TripleOp promoter used extensively in plants (Gatz et al. (1992) Plant J 2:397). However, alternative configurations are possible, including the number, placement, and/or sequence of tet operators used to design ligand-regulated promoters, which can be varied based on function, promoter type, promoter sequence, promoter conservation, species, and other criteria (see, e.g., Berens & Hillen (2003) Eur J Biochem 270:3109; Gatz & Quail (1998) PNAS 85:1394-1397; Gatz et al. (1991) Mol Gen Genet 227:229-237; Frohberg et al. (1991) PNAS 88:10470-10474).

Selected promoters were evaluated by analyzing one or more related candidate promoters to identify any conserved regions, and to locate motifs including TATA-box, Y-patches, and transcriptional start site(s) (TSS). In some examples, one or more of these motifs could not be unambiguously identified. The objective was to incorporate the tet operator sequences for maximal predicted function, while minimizing disruptions to sequence, conserved regions, motifs, and spacing between motifs and/or conserved regions. Two operator sequences were incorporated flanking the predicted TATA box, and the third operator is near or overlapping with the transcription start site. The final location and spacing of the operators depends on the promoter sequence and motifs. When possible, operator sequences are placed to minimize changes, and therefore will be sequence replacements rather than insertions. After designing the placement of operators into the promoter, the resulting sequences were re-analyzed to confirm that the original promoter motifs are predicted by the analysis methods and algorithms.

Generally, tet operator sequences were placed within a few nucleotides of either side of the TATA box, and in some cases there was a short overlap with the TATA box sequence or the transcription start site (TSS). A third operator was placed downstream from the second operator near the transcription start site. The third operator was typically downstream of the TSS, and in some instances had some overlap with the TSS sequence. Activity from these promoters can be controlled using any tetracycline compound/tetracycline repressor system, and/or using any sulfonylurea compound/sulfonylurea repressor system.

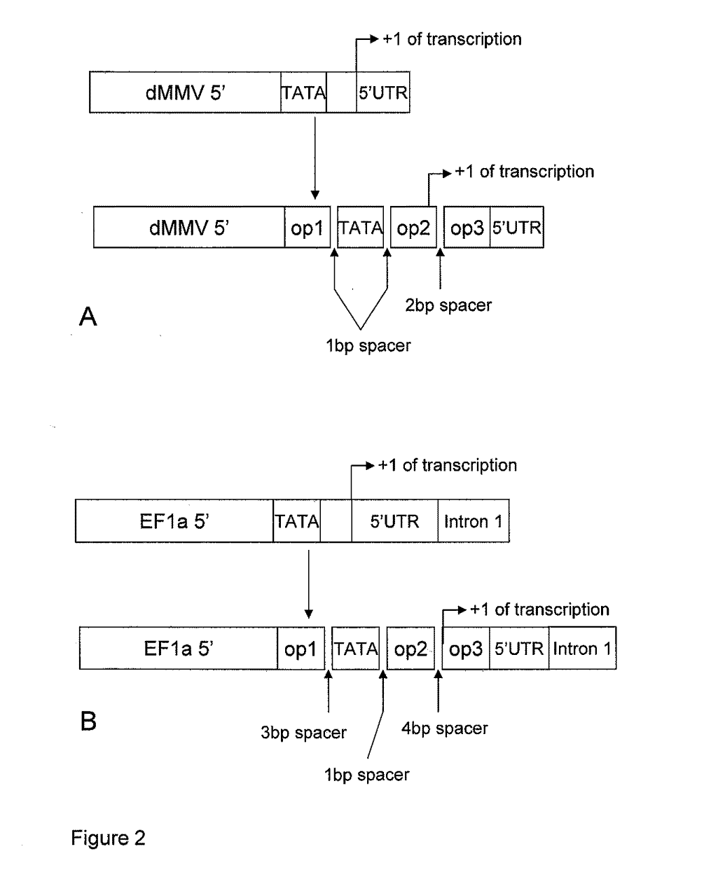

Mirabilis mosaic virus (MMV) promoters with single (SEQ ID NO:851) and double enhancer domains (dMMV) (SEQ ID NO:852) (Dey & Maiti (1999) Plant Mol Biol 40:771-782; Dey & Maiti (1999) Transgenics 3:61-70) were analyzed to identified conserved sequence regions and putative motifs. These promoter sequences were modified as generally described above to avoid disrupting any conserved region or motif and to include three copies of tet operator (SEQ ID NO:848) to produce regulated promoters MMV::tetOp (SEQ ID NO:857) and dMMV::tetOp (SEQ ID NO:858). The promoter design is shown in FIG. 2A. The second tet operator has a small overlap with the predicted transcription start site (TSS). Activity from these promoters can be controlled using any tetracycline compound/tetracycline repressor system, and/or using any sulfonylurea compound/sulfonylurea repressor system.

Banana streak virus Acuminata Yunan (BSV(AY)) promoter (SEQ ID NO:850) was analyzed with six other BSV isolate promoter sequences in order to identify conserved sequence regions and putative motifs. The analysis identified several conserved regions, and putative TATA box, TSS, and Y-patches. BSV(AY) promoter sequence was modified as generally described above to include three copies of tet operator (SEQ ID NO:848) to produce a regulated promoter BSV::tetOp (SEQ ID NO:856). The designed sequence was re-analyzed regarding the predicted TATA box and TSS.

An EF1A2 promoter from Glycine max (SEQ ID NO:854) was analyzed with another soybean, two Arabidopsis, and two Medicago EF1A promoter sequences to identify conserved sequence regions and motifs. Based on this analysis, placement of three copies of tet operator (SEQ ID NO:848) was designed to produce regulated promoter EF1A2::tetOp (SEQ ID NO:860). The designed promoter was re-analyzed to confirm retention of the previously identified TATA box and TSS. The promoter design is shown in FIG. 2A.

An Oryza sativa actin promoter (SEQ ID NO:849) was analyzed with an actin promoter from Zea mays and one from Sorghum bicolor. Based on this analysis, placement of three copies of tet operator (SEQ ID NO:848) was designed to produce regulated promoter OsActin::tetOp (SEQ ID NO:855). The designed promoter was re-analyzed regarding previously identified motifs including the TATA box and the TSS.

MPSS data and EST distribution were used to develop a short-list of promoters that appeared to be expressed in the maize meristem and were not active in callus. MPSS data identified 92 potential meristem-specific genes. Seven of these had putative TATA boxes within their promoters. Two of the seven were represented by EST's, only one of which had a well-defined TATA box. This promoter, which drives expression of a maize p450 gene, was designated MP1 (SEQ ID NO:853). The full length maize, sorghum and rice MP1 promoters were analyzed as described above in order to identify conserved sequences, motifs, TATA box, and transcription start site. Three copies of tet operator sequence (SEQ ID NO:848) were positioned flanking the TATA box and just downstream of the transcription start site, taking care to avoid conserved motifs to produce regulated promoter MP1:tetOp (SEQ ID NO:859). The designed promoter was re-analyzed to confirm retention of the previously identified TATA box and TSS.

The activity of unmodified and designed MMV, dMMV, and EF1A2 promoters were evaluated using the characterized 35SCaMV (SEQ ID NO:861) and 35SCaMV::tetOP (SEQ ID NO:862) promoters as controls. Promoter activity was analyzed via Agrobacterium-mediated transient expression analysis in Nicotiana benthamiana leaves. N. benthamiana leaves were infected with luciferase reporter constructs controlled by either the modified or unmodified promoters. Test constructs were identical except for the promoter sequence (Pro) being tested. Test constructs comprised the following operably linked components:
RB-Pro-RLuc-UBQ3-EF1A-NptII-EF1A3′-LB

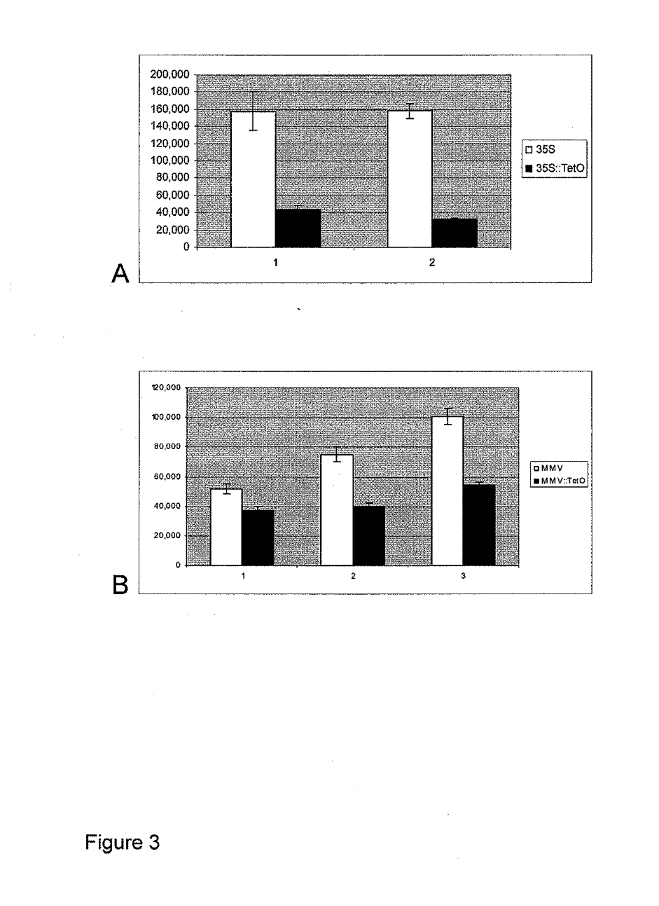

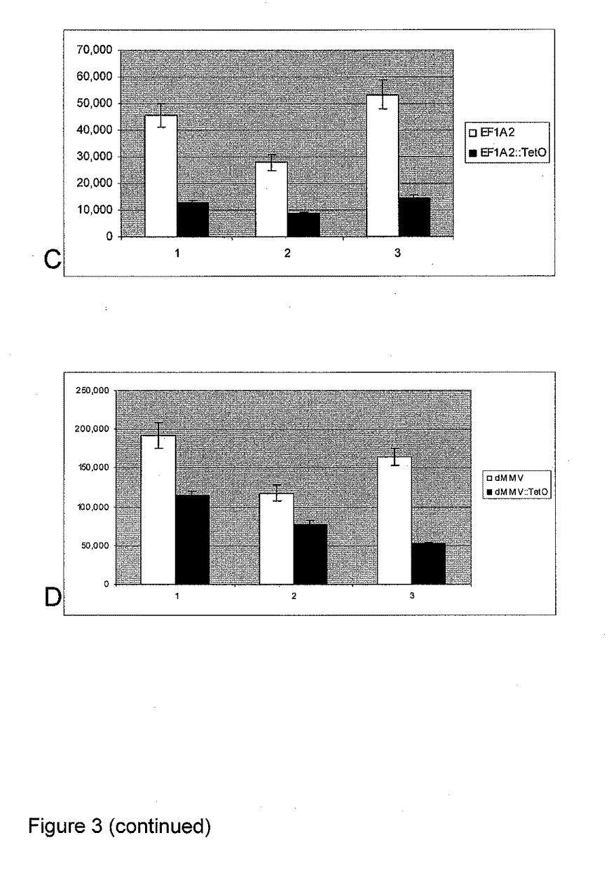

Relative light units were quantified for modified and unmodified promoters (FIG. 3), and promoter activity for the modified version determined as the percent of unmodified promoter activity (ProOp/Pro). The results show that all modified promoters are still active but do suffer some reduction in activity as a result of the added tet operator sequences. In this assay, 35S:tetOp had about 25% of the activity as 35S promoter, EF1A2:tetOp had about 30% of the activity of EF1A2 promoter, MMV:tetOp had about 60% of the activity of MMV promoter, and dMMV:tetOp had about 50% of the activity of dMMV promoter.

Sulfonylurea compound/SuR regulation of these same promoters was analyze by co-infecting N. benthamiana leaves with the above test constructs and with an Agrobacterium strain comprising a sulfonylurea repressor expression construct (pVER7555):
RB-dMMV-SuR-UBQ14-EF1A-NptII-ScCAL1 3′-LB

or a control construct (pVER7549):
RB-dPCSV-FLuc-UBQ3-EF1A-NptII-EF1A3′-LB

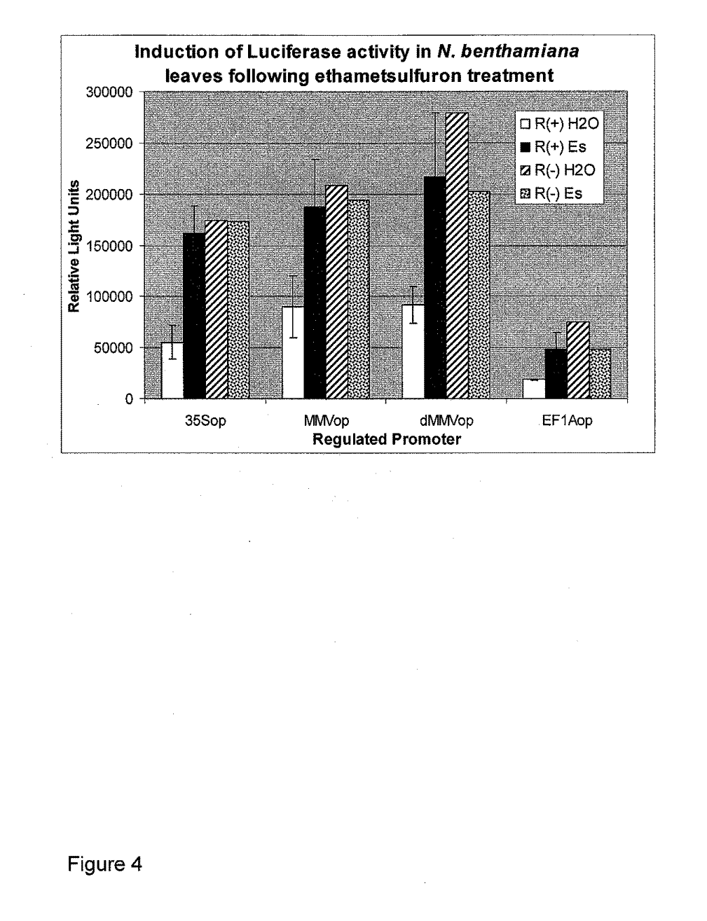

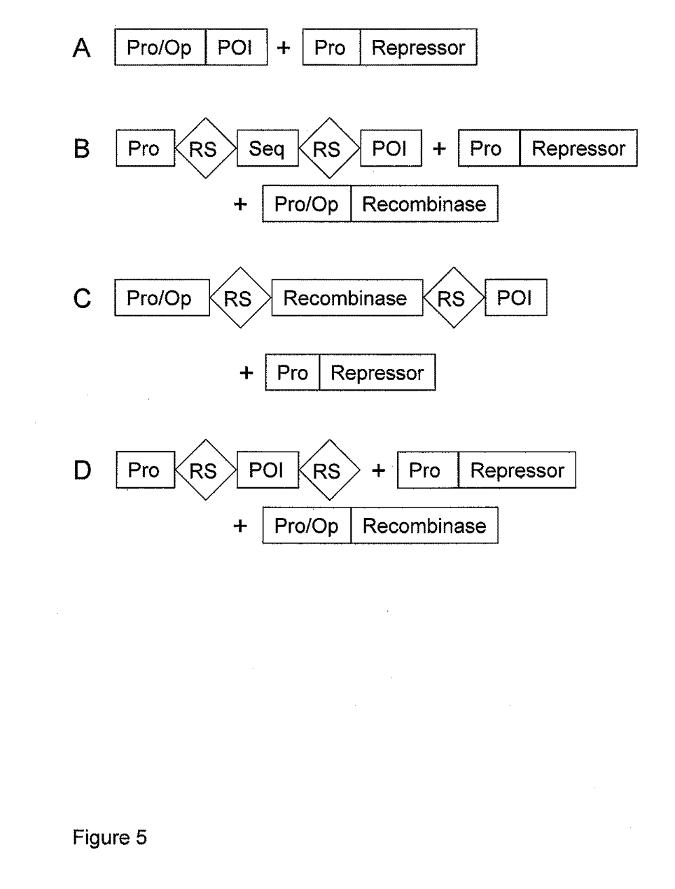

Repression and de-repression were tested with control (H₂O) and sulfonylurea ligand (ethametsulfuron, Es). The data demonstrate that all promoters, including the control 35S::tetO promoter, are repressed and de-repressed to a similar degree (FIG. 4).

The BSV::tetOp promoter and the MP1::tetOp promoter were synthesized and cloned into expression cassettes driving expression of the DsRED (RFP) gene for testing. For all comparisons of RFP expression, side-by-side comparisons were made within experiment by taking micrographs of the tissue at the same exposure and qualitatively ranking fluorescence intensity, assigning the scores in the table below. The DsRED protein is very stable (has a relatively long half-life), and even with low expression levels in the cell can accumulate over time. Thus, when no fluorescence or low levels of fluorescence were observed in the absence of ligand, this likely represented a transgenic event with relatively stringent repression.

| Score | Description |
|---|---|
| 0 | no fluorescence |
| 1 | faint fluorescence |
| 2 | medium intensity |
| 3 | strong fluorescence |
| 4 | very bright fluorescence |

Maize immature embryos were transformed with Agrobacterium as generally described in Example 5D. Transformants were generated that contained one of the two T-DNAs shown below:
RB-BSV:tetOp::Adh Intron::dsRed+ALS:HRA+Ubi:ESR(3E3)-LB
RB-35S:tetOp::Adh intron::dsRed+ALS:HRA+Ubi::ESR(3E3)-LB

Selection of transgenic events was performed using 30, 100, or 500 μg/L chlorsulfuron. Chlorsulfuron is generally a less active inducer of the ethametsulfuron repressor, having approximately 10-fold less activity. After six weeks on these three different levels of chlorsulfuron, very low levels of inducible RFP was observed in the 30 μg/l treatment (Score=0. In the 100 μg/l treatment small sectors of RFP fluorescence were observed, and although the brightness of the fluorescence was stronger than for the 30 μg/L treatment, it was still relatively weak. In the 500 μg/l treatment, large segments of the calli were brightly fluorescing. Thus, with increasing concentrations of ligand during cell culture, a corresponding increase in RFP de-repression was observed.

### Example 8

### Auto-Regulation of Gene Switch

Any combination of gene switch elements can be used, including but not limited to one or more of the sulfonylurea-responsive repressors, tetracycline-responsive repressors, or tetracycline operator-containing promoters provided.

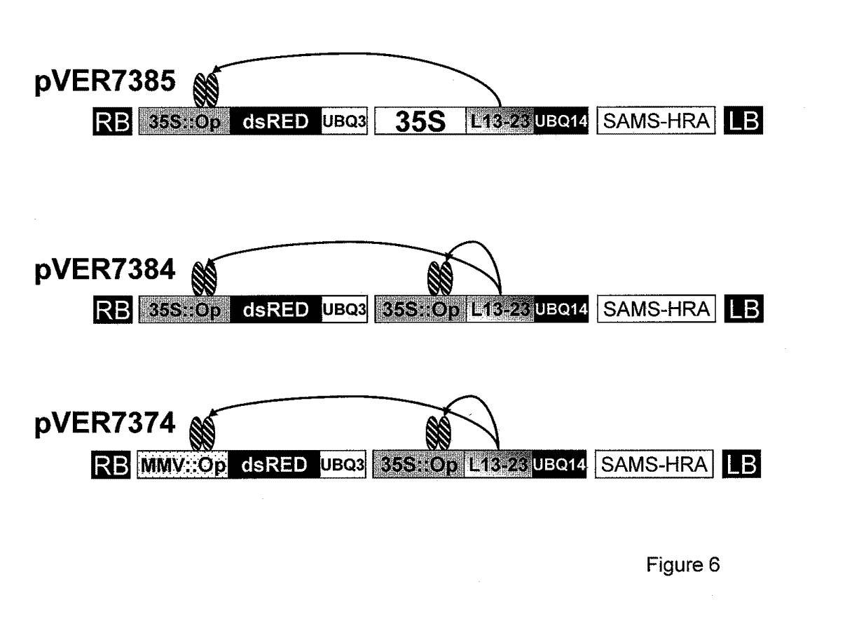

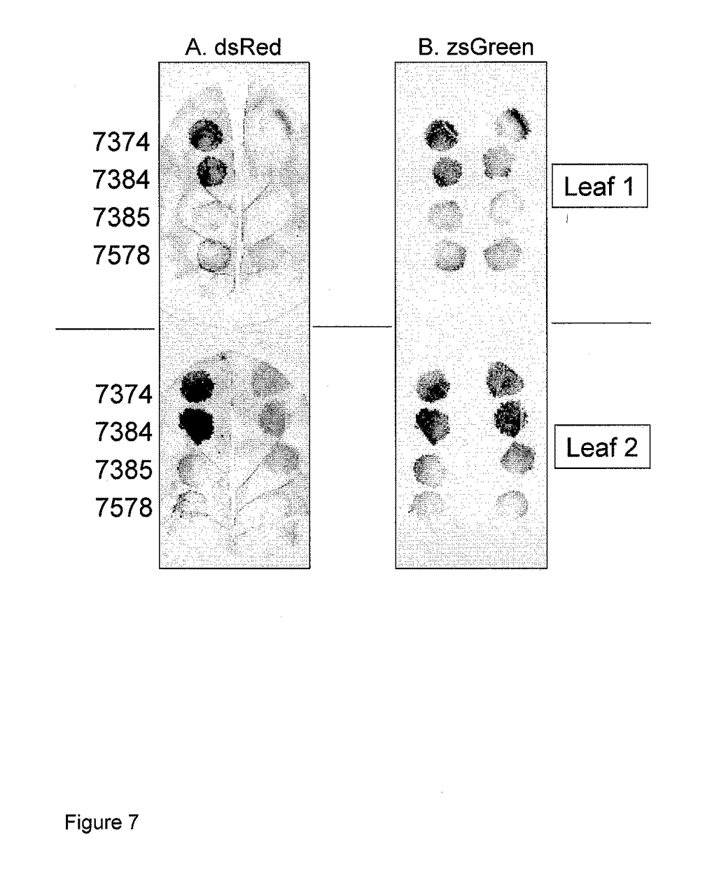

To determine if auto-regulation would enhance ligand-induced plant gene expression, transformation vectors for comparing regulation of dsRED from auto-regulated or constitutively expressed EsR (L13-23) were constructed and tested in transient expression assays. The standard vector, pVER7385 (35SOp-dsRED-UBQ3/35S-EsR(L13-23)-UBQ14/SAMS-HRA), expresses dsRED from the 35SOp promoter and the repressor from a constitutive 35S promoter. The auto-regulated test vector, pVER7384, is essentially the same as pVER7385 except that both dsRED and EsR are controlled by the same 35SOp promoter. A second auto-regulated test vector, pVER7374, differs from pVER7384 in that the regulated MMV::Op promoter described in Example 7 was used in place of the 35SOp promoter (FIG. 6). An additional control vector, pVER7578 (35SOp-dsRED-UBQ3/dMMV-EsR(L7-A11)-UBQ14/SAMS-HRA), was included and was previously shown to deliver inducible dsRED expression in N. benthamiana leaves. This vector differs from pVER7385 in that the strong dMMV promoter drives expression of repressor L7-A11. Construction of all vectors was performed using standard cloning techniques. Each vector was transformed into Agrobacterium tumefaciens AGL1 and then subjected to transient expression analysis in N. benthamiana by forced leaf infiltration. For each sample, the test strain was mixed in an 80:20 ratio with a strain bearing a constitutive ZsGREEN vector used to normalize expression. Each sample was infiltrated once per half leaf, repeated on each half of the leaf, and the same pattern repeated on a separate leaf. Following 2 days co-cultivation, leaves were excised from the plant, cut down the vein line and the cut edge placed into either H₂O or 1 ppm Ethametsulfuron for two days. All samples were exposed in parallel to water or inducer treatments and repeated once. Following treatment the leaves were imaged for dsRED and zsGREEN expression using a Typhoon Laser Scanner (GE Healthcare, Piscataway, N.J.). DsRED (BD Sciences, Clontech) expression was quantified using an excitation wavelength of 532 nm and an emission wavelength of 580 nM. ZsGREEN (BD Sciences, Clontech) expression was quantified using an excitation wavelength of 488 nm and an emission wavelength of 520 nm. The data show induction of dsRED is more robust from vectors expressing an auto-regulated repressor (FIG. 7). The data also show that it is not essential to have the same promoter regulating both dsRED and the repressor to achieve enhanced induction.

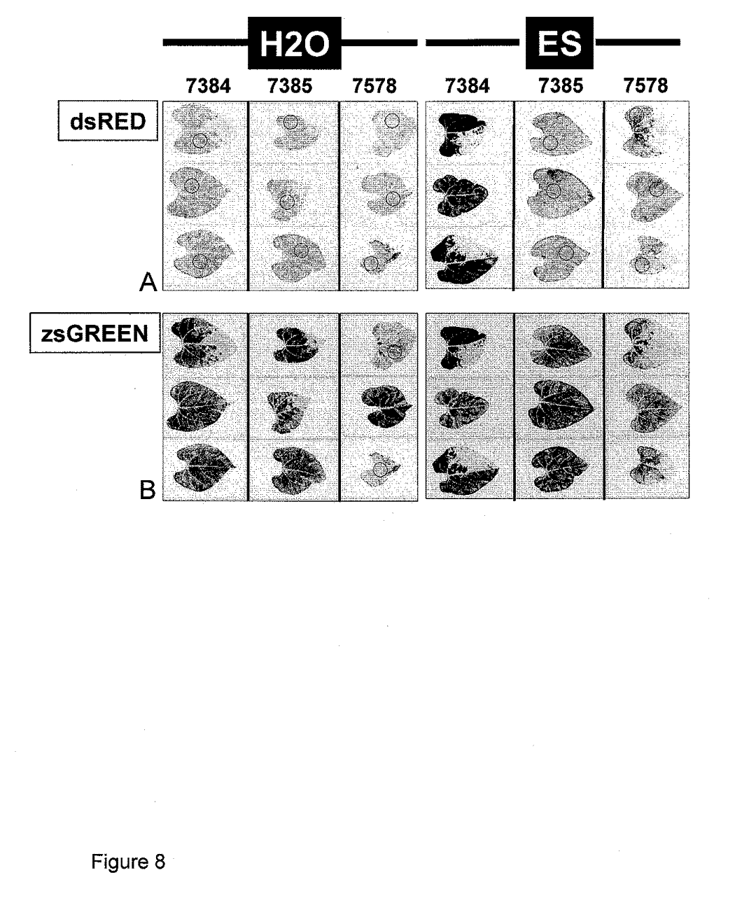

A second transient assay was performed by vacuum infiltration of test Agrobacterium cultures into the first true leaves of Phaseolus vulgaris. This was done by submerging the entire leaf bearing section of the plant into beakers of test culture composed of a 50:50 mix of the test strain with a strain bearing a constitutive ZsGREEN vector used to normalize expression. In this experiment the test was limited to vectors pVER7384, pVER7385, and pVER7578. The results show that the auto-regulated construct pVER7384 is induced better than for either of the two standard vectors (FIG. 8). ZsGREEN expression patterns of the same leaf samples indicate that this effect cannot be accounted for by variance in overall expression competency of the infiltrated leaf. In addition, the results demonstrate that the effect of auto-regulation on ligand-induced plant gene expression is not unique to one test plant system and is likely to be universal for all plant expression hosts.

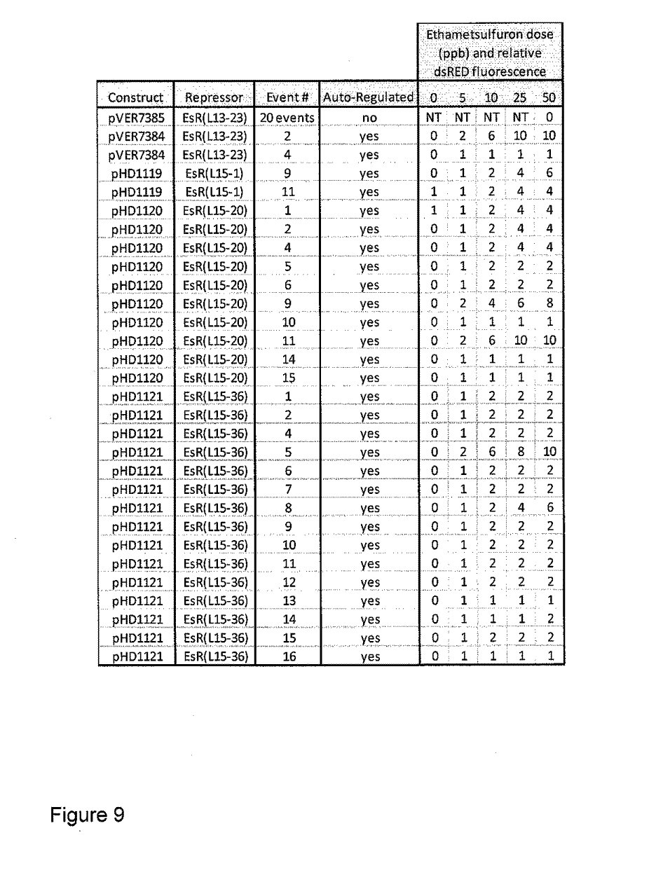

To establish that this phenomenon also applied to transgenic plants, tobacco was transformed with test vectors and plant leaf tissue was analyzed for sensitivity to inducer. Plasmids pVER7384 and pVER7385 described above were used to test auto-regulation vs. constitutive repressor expression using the same shuffled repressor variant: EsR(L13-23). Plasmids pHD1119, pHD1120, and pHD1121 are essentially the same as pVER7384 except they encode shuffled repressor variants EsR(L15-1), EsR(L15-20) and EsR(L15-36). Construction of all vectors was performed using standard cloning techniques. All vectors were transformed into disarmed Agrobacterium tumefaciens EHA105 and each new strain subsequently used to co-cultivate 64 leaf explants of Nicotiana tabacum ‘Petite Havana’. Following co-cultivation the tissues were placed on medium with 50 ppb imazapyr to select for the presence of the linked ‘SAMS-HRA’ gene which encodes an allele of acetolactate synthase that is cross resistant to both sulfonylurea and imidizolinone herbicides. Imazapyr was used as the selective agent instead of sulfonylurea compounds since this herbicide will not induce the switch yet still act as a selective agent. Transformed shoots arising from each co-cultivation experiment are analyzed for their level of leaky dsRED expression. Only those lines with no (or minimal) leaky dsRED expression were carried forward for induction analysis. Leaf disks from each event (except for those arising from pVER7385) were placed on 0, 5, 10, 25, and 50 ppb ethametsulfuron (Es) and incubated in the light at 25° C. Leaf disks arising from transformed events from the pVER7385 co-cultivation were placed on 0 and 50 ppb ethametsulfuron and incubated in parallel with the other samples. After 24 hours of incubation, the leaf disks were imaged and scored for relative dsRED expression. As demonstrated in FIG. 9, none of the pVER7385 (repressor not auto-regulated) leaf pieces express significant dsRED activity at 50 ppb Es whereas all the samples arising from an auto-regulated repressor showed some degree of derepression, starting at as little as 5 ppb inducer and some intensely expressing at just 10 ppb Es. The results demonstrate that the effects of auto-regulation on ligand-induced plant gene expression are reproducible in transgenic plants.

The articles “a” and “an” refer to one or more than one of the grammatical object of the article. By way of example, “an element” means one or more of the element. All book, journal, patent publications and grants mentioned in the specification are indicative of the level of those skilled in the art. All publications and patent applications are herein incorporated by reference to the same extent as if each individual publication or patent application was specifically and individually indicated to be incorporated by reference. Although the foregoing invention has been described in some detail by way of illustration and example for purposes of clarity of understanding, certain changes and modifications may be practiced within the scope of the appended claims. These examples and descriptions are illustrative and are not read as limiting the scope of the appended claims.

What is claimed:

1. A method to regulate expression in a plant cell contained in a plant or in a seed comprising:
(a) providing the plant cell comprising a sulfonylurea-regulated gene switch which controls expression of a polynucleotide of interest, wherein said plant cell is a sulfonylurea tolerant plant cell; and;
(b) providing a sulfonylurea compound which regulates the gene switch wherein the sulfonylurea compound is provided by foliar application, root drench application, pre-emergence application, post-emergence application, or seed treatment application.

2. The method of claim 1, wherein expression of the polynucleotide of interest alters the phenotype of the plant cell.

3. The method of claim 1, wherein expression of the polynucleotide of interest alters the genotype of the plant cell.

4. The method of claim 1, wherein providing the sulfonylurea compound activates expression of the polynucleotide of interest.

5. The method of claim 1, wherein the plant cell is from a monocot or a dicot.

6. The method of claim 5, wherein the plant cell is from maize, rice, sorghum, sugarcane, barley, oat, wheat, turfgrass, soybean, canola, cotton, tobacco, sunflower, safflower, or alfalfa.

7. The method of claim 1, wherein the sulfonylurea-regulated gene switch comprises a repressible promoter operably linked to a polynucleotide of interest, wherein the repressible promoter comprises a tet operator.

8. The method of claim 1, wherein the sulfonylurea-regulated gene switch comprises a repressible promoter selected from the group consisting of SEQ ID NO:855, 856, 857, 858, 859, and 860, or a repressible promoter having at least 95% sequence identity to SEQ ID NO:855, 856, 857, 858, 859, 860 or 862.

9. The method of claim 1, wherein the sulfonylurea compound comprises a pyrimidinylsulfonylurea compound, a triazinylsulfonylurea compound, or a thiadazolylurea compound.

10. The method of claim 9, wherein the sulfonylurea compound comprises a chlorosulfuron, an ethametsulfuron, a thifensulfuron, a metsulfuron, a sulfometuron, a tribenuron, a chlorimuron, a nicosulfuron, or a rimsulfuron.

11. The method of claim 1, wherein the polynucleotide of interest encodes a polypeptide that specifically binds to a target nucleic acid sequence.

12. The method of claim 11, wherein the polypeptide is a recombinase, an integrase, a nuclease, a homing endonuclease, or a zinc-finger nuclease.

13. The method of claim 1, wherein the sulfonylurea-regulated gene switch comprises a sulfonylurea-responsive repressor comprising an amino acid sequence of any one of SEQ ID NOs:3-419, or an amino acid sequence having at least 85% sequence identity to any one of SEQ ID NOs:3-419.

14. A sulfonylurea-regulated gene switch which controls expression of a polynucleotide of interest, wherein the polynucleotide of interest encodes a polypeptide that specifically binds a DNA sequence or encodes a polypeptide that cuts a DNA sequence.

15. The sulfonylurea-regulated gene switch of claim 14, wherein the encoded polypeptide is a recombinase, an integrase, a nuclease, a homing endonuclease, or a zinc-finger nuclease.

16. The sulfonylurea-regulated gene switch of claim 14, wherein the sulfonylurea gene switch comprises a sulfonylurea responsive repressor, wherein the sulfonylurea responsive repressor is operably linked to a promoter active in the plant wherein the promoter is a constitutive promoter, a tissue-preferred promoter, a developmental stage-preferred promoter, an inducible promoter, or a repressible promoter.

17. The sulfonylurea-regulated gene switch of claim 16, wherein
(a) the constitutive promoter is a MTH promoter, an EF1a promoter, a PIP promoter, a ubiquitin promoter, an actin promoter, or a 35S CaMV promoter;
(b) the tissue-preferred promoter is a meristem-preferred promoter, an embryo-preferred promoter, a leaf-preferred promoter, a root-preferred promoter, an anther-preferred promoter, a pollen-preferred promoter, or a floral-preferred promoter;
(c) the developmental stage-preferred promoter is an early embryo promoter, a late embryo promoter, a germination-preferred promoter, or a senescence-preferred promoter;
(d) the inducible promoter is a chemical inducible promoter, a pathogen inducible promoter, a heat-stress promoter, a drought-stress promoter, a light inducible promoter, an osmoticum inducible promoter, or a metal inducible promoter; or,
(e) the repressible promoter is a tetracycline-repressible promoter, or a lactose-repressible promoter.

18. The sulfonylurea-regulated gene switch of claim 16, wherein the polynucleotide of interest or said sulfonylurea-responsive repressor is operably linked to a repressible promoter comprising at least one tetracycline operator sequence.

19. The sulfonylurea-regulated gene switch of claim 18, wherein the repressible promoter is a constitutive promoter, a tissue-preferred promoter, or a development stage-preferred promoter.

20. The sulfonylurea-regulated gene switch of claim 19, wherein the repressible promoter is a 35S CaMV promoter, an actin promoter, an EF1A promoter, an MMV promoter, a dMMV promoter, a MP1 promoter, or a BSV promoter.

21. The sulfonylurea regulated gene switch of claim 20, wherein the repressible promoter comprises a polynucleotide sequence as set forth in SEQ ID NO:855, 856, 857, 858, 859, 860 or 862, or a polynucleotide sequence having at least 95% sequence identity to SEQ ID NO:855, 856, 857, 858, 859, 860 or 862.

22. The sulfonylurea regulated gene switch of claim 16, wherein the sulfonylurea-responsive repressor comprises an amino acid sequence of any one of SEQ ID NOs:3-419, or an amino acid sequence having at least 85% sequence identity to any one of SEQ ID NOs:3-419.

23. A transgenic plant, a transgenic plant cell, or a transgenic seed comprising the sulfonylurea regulated gene switch of claim 14.

24. The transgenic plant, the transgenic plant cell, or the transgenic seed of claim 23, wherein the transgenic plant, the transgenic plant cell, or the transgenic seed is from a monocot or a dicot.

25. The transgenic plant, the transgenic plant cell, or the transgenic seed of claim 24, wherein the monocot or dicot is from maize, rice, sorghum, sugarcane, barley, oat, wheat, turfgrass, soybean, canola, cotton, tobacco, sunflower, safflower, or alfalfa.

26. A recombinant polynucleotide comprising a repressible promoter, wherein the repressible promoter is active in a plant cell, and the repressible promoter comprises an actin promoter, an EF1A promoter, an MMV promoter, a dMMV promoter, an MP1 promoter, or a BSV promoter operably linked to at least one operator sequence.

27. The recombinant polynucleotide of claim 26, wherein the repressible promoter comprises a polynucleotide sequence as set forth in SEQ ID NO:855, 856, 857, 858, 859, 860 or 862 or a polynucleotide sequence having at least 95% sequence identity to SEQ ID NO:855, 856, 857, 858, 859, 860 or 862.

28. A method to regulate expression in a cell comprising:
(a) providing the cell comprising a regulated gene switch which controls expression of a polynucleotide of interest, wherein the gene switch comprises a repressible promoter of claim 26; and;
(b) providing a ligand compound which regulates the gene switch, wherein ligand compound comprises a tetracycline, a sulfonylurea, or any analogs thereof.

29. A recombinant polynucleotide comprising a repressible promoter operably linked to a polynucleotide encoding a sulfonylurea-responsive repressor.

30. The recombinant polynucleotide of claim 29, wherein the encoded sulfonylurea-responsive repressor comprises an amino acid sequence of any one of SEQ ID NOs:3-419 or an amino acid sequence having at least 85% sequence identity to any one of SEQ ID NOs:3-419.

31. The recombinant polynucleotide of claim 29, wherein the repressible promoter is an actin promoter, an MMV promoter, a dMMV promoter, an MP1 promoter, or a BSV promoter operably linked to at least one operator sequence.

32. The recombinant polynucleotide of claim 31, wherein the repressible promoter comprises a polynucleotide sequence as set forth in SEQ ID NO:855, 856, 857, 858, 859, 860 or 862, or a polynucleotide sequence having at least 95% sequence identity to SEQ ID NO:855, 856, 857, 858, 859, 860 or 862.
